SEQUENCE LISTING (92 sequences)

SEQ ID NO: 1 - protein, 11 aa, synthetic construct
RASQSVSSYLA

SEQ ID NO: 2 - protein, 7 aa, synthetic construct
DASNRAT

SEQ ID NO: 3 - protein, 9 aa, synthetic construct
QQRSNWPPT

SEQ ID NO: 4 - protein, 5 aa, synthetic construct
NYGMH

SEQ ID NO: 5 - protein, 17 aa, synthetic construct
VIYYDGSNKFYADSVKG

SEQ ID NO: 6 - protein, 14 aa, synthetic construct
DVAPPGSRAEYFHH

SEQ ID NO: 7 - protein, 5 aa, synthetic construct
RYEMN

SEQ ID NO: 8 - protein, 17 aa, synthetic construct
YINSIGSAINYADSVKG

SEQ ID NO: 9 - protein, 8 aa, synthetic construct
EDNWNPGY

SEQ ID NO: 10 - protein, 5 aa, synthetic construct
NYGMH

SEQ ID NO: 11 - protein, 17 aa, synthetic construct
VIWYDGRNKYYADSVKG

SEQ ID NO: 12 - protein, 14 aa, synthetic construct
DVAPAGSRAEYFQN

SEQ ID NO: 13 - protein, 5 aa, synthetic construct
SYGMH

SEQ ID NO: 14 - protein, 17 aa, synthetic construct
VIFYDGSNKYYADSVKG

SEQ ID NO: 15 - protein, 14 aa, synthetic construct
DVAAPGSRAEYFQN

SEQ ID NO: 16 - protein, 5 aa, synthetic construct
NYGMH

SEQ ID NO: 17 - protein, 17 aa, synthetic construct
IIYYDGSNKYYADSVKG

SEQ ID NO: 18 - protein, 14 aa, synthetic construct
DVAAAGSRAEYLHH

SEQ ID NO: 19 - protein, 5 aa, synthetic construct
SYWIS

SEQ ID NO: 20 - protein, 17 aa, synthetic construct
RIDPSDSYTNYNPSFQG

SEQ ID NO: 21 - protein, 10 aa, synthetic construct
RDGSYFDFDY

SEQ ID NO: 22 - protein, 5 aa, synthetic construct
SYWIS

SEQ ID NO: 23 - protein, 17 aa, synthetic construct
RIDPSDSYTKYSPSFQG

SEQ ID NO: 24 - protein, 10 aa, synthetic construct
RDGDYFDFDY

SEQ ID NO: 25 - protein, 7 aa, synthetic construct
GFTFSNY

SEQ ID NO: 26 - protein, 6 aa, synthetic construct
YYDGSN

SEQ ID NO: 27 - protein, 14 aa, synthetic construct
DVAPPGSRAEYFHH

SEQ ID NO: 28 - protein, 7 aa, synthetic construct
GFTFSRY

SEQ ID NO: 29 - protein, 6 aa, synthetic construct
NSIGSA

SEQ ID NO: 30 - protein, 8 aa, synthetic construct
EDNWNPGY

SEQ ID NO: 31 - protein, 7 aa, synthetic construct
GFTFSNY

SEQ ID NO: 32 - protein, 6 aa, synthetic construct
WYDGRN

SEQ ID NO: 33 - protein, 14 aa, synthetic construct
DVAPAGSRAEYFQN

SEQ ID NO: 34 - protein, 7 aa, synthetic construct
GFTFSSY

SEQ ID NO: 35 - protein, 6 aa, synthetic construct
FYDGSN

SEQ ID NO: 36 - protein, 14 aa, synthetic construct
DVAAPGSRAEYFQN

SEQ ID NO: 37 - protein, 7 aa, synthetic construct
GFTFSNY

SEQ ID NO: 38 - protein, 6 aa, synthetic construct
YYDGSN

SEQ ID NO: 39 - protein, 14 aa, synthetic construct
DVAAAGSRAEYLHH

SEQ ID NO: 40 - protein, 7 aa, synthetic construct
GYSFTSY

SEQ ID NO: 41 - protein, 6 aa, synthetic construct
DPSDSY

SEQ ID NO: 42 - protein, 10 aa, synthetic construct
RDGSYFDFDY

SEQ ID NO: 43 - protein, 7 aa, synthetic construct
GYSFTSY

SEQ ID NO: 44 - protein, 6 aa, synthetic construct
DPSDSY

SEQ ID NO: 45 - protein, 10 aa, synthetic construct
RDGDYFDFDY

SEQ ID NO: 46 - protein, 123 aa, synthetic construct
QVQLVESGGGVVQPGRSLRLSCAASGFTFSNYGMHWVRQAPGKGLEWVAVIYYDGSNKFYADSVKGRFTISRDNSKNTLYLQMSSLRAEDTAVYYCARDVAPPGSRAEYFHHWGQGTLVTVSS

SEQ ID NO: 47 - protein, 117 aa, synthetic construct
QVQLVQSGGGLVQPGGSLRLSCAASGFTFSRYEMNWVRQAPGKGLEWVSYINSIGSAINYADSVKGRFTISRDNAKNSLYLQMRSLRAEDTAVYYCVREDNWNPGYWGQGTLVTVSS

SEQ ID NO: 48 - protein, 123 aa, synthetic construct
QVQLVQSGGGVVQPGRSLRLSCAASGFTFSNYGMHWVRQAPGKGLEWVAVIWYDGRNKYYADSVKGRFTISRDNSKNTLYLQMNSLRAEDTAVYYCARDVAPAGSRAEYFQNWGQGTLVTVSS

SEQ ID NO: 49 - protein, 123 aa, synthetic construct
QVQLVESGGGVVQPGRSLRLSCAASGFTFSSYGMHWVRQAPGKGLEWVAVIFYDGSNKYYADSVKGRFTISRDNSKNTLYLQMNSLRAEDTSVYYCARDVAAPGSRAEYFQNWGQGTLVTVSS

SEQ ID NO: 50 - protein, 123 aa, synthetic construct
QVQLVESGGGVVQPGRSLRLSCVASGFTFSNYGMHWVRQAPGKGLEWVAIIYYDGSNKYYADSVKGRFTISRDNSKNTLYLQMNSLRAEDTAVYYCARDVAAAGSRAEYLHHWGQGTLVTVSS

SEQ ID NO: 51 - protein, 119 aa, synthetic construct
QVQLEQSRAEVKKPGESLRISCKGSGYSFTSYWISWVRQMPGKGLEWMGRIDPSDSYTNYNPSFQGHVTISADKSISTAYLQWSSLKASDTAMYYCARRDGSYFDFDYWGQGTLVTVSS

SEQ ID NO: 52 - protein, 119 aa, synthetic construct
QVQLVESGAEVKKPGESLRISCRGSGYSFTSYWISWVRQMPGKGLEWMGRIDPSDSYTKYSPSFQGHVTISADKSISTAYLQWSSLKASDTAMYYCARRDGDYFDFDYWGQGTLVTVSS

SEQ ID NO: 53 - protein, 107 aa, synthetic construct [sequence omitted]

SEQ ID NO: 54 - protein, 7 aa, synthetic construct
SGYYYWG

SEQ ID NO: 55 - protein, 16 aa, synthetic construct
SIYYIGTTYYIPSLKS

SEQ ID NO: 56 - protein, 13 aa, synthetic construct
QPITVAGHDAFDI

SEQ ID NO: 57 - protein, 7 aa, synthetic construct
SSYYFWG

SEQ ID NO: 58 - protein, 16 aa, synthetic construct
AIYYIGNTHYNPSLKS

SEQ ID NO: 59 - protein, 10 aa, synthetic construct
YEDTWNWFDP

SEQ ID NO: 60 - protein, 5 aa, synthetic construct
YYYWS

SEQ ID NO: 61 - protein, 16 aa, synthetic construct
NIYYSGDTNYNPSLRS

SEQ ID NO: 62 - protein, 13 aa, synthetic construct
YDMFKWNYEAFDV

SEQ ID NO: 63 - protein, 9 aa, synthetic construct
GGSIDSGYY

SEQ ID NO: 64 - protein, 5 aa, synthetic construct
YYIGT

SEQ ID NO: 65 - protein, 13 aa, synthetic construct
QPITVAGHDAFDI

SEQ ID NO: 66 - protein, 9 aa, synthetic construct
GGSISSSYY

SEQ ID NO: 67 - protein, 5 aa, synthetic construct
YYIGN

SEQ ID NO: 68 - protein, 10 aa, synthetic construct
YEDTWNWFDP

SEQ ID NO: 69 - protein, 7 aa, synthetic construct
GGSISYY

SEQ ID NO: 70 - protein, 5 aa, synthetic construct
YYSGD

SEQ ID NO: 71 - protein, 13 aa, synthetic construct
YDMFKWNYEAFDV

SEQ ID NO: 72 - protein, 123 aa, synthetic construct
EVQLVQSGPGLVKPSETLSLTCTVSGGSIDSGYYYWGWIRQPPGKGLEWIGSIYYIGTTYYIPSLKSRVTISVDTSKNRFSLKLTSVTAADTAVYYCARQPITVAGHDAFDIWGQGTMVTVSS

SEQ ID NO: 73 - protein, 120 aa, synthetic construct
QLQLQQSGPGLLKPSETLSLTCTVSGGSISSSYYFWGWIRQPPGKGLEWIGAIYYIGNTHYNPSLKSRVTISIDTSKNQFSLNLNSVTAADTAIYYCARYEDTWNWFDPWGQGTLVTVSS

SEQ ID NO: 74 - protein, 121 aa, synthetic construct
QLQLKESGPGLVKPSETLSLTCTVSGGSISYYYWSWIRQPPGMGLEWIGNIYYSGDTNYNPSLRSRVTISIDMSKNQFSLRLNSVTAADTAVYYCARYDMFKWNYEAFDVWGQGTMVTVSS

SEQ ID NO: 75 - protein, 1070 aa, Homo sapiens
MGAARGSPARPRRLPLLSVLLLPLLGGTQTAIVFIKQPSSQDALQGRRALLRCEVEAPGPVHVYWLLDGAPVQDTERRFAQGSSLSFAAVDRLQDSGTFQCVARDDVTGEEARSANASFNIKWIEAGPVVLKHPASEAEIQPQTQVTLRCHIDGHPRPTYQWFRDGTPLSDGQSNHTVSSKERNLTLRPAGPEHSGLYSCCAHSAFGQACSSQNFTLSIADESFARVVLAPQDVVVARYEEAMFHCQFSAQPPPSLQWLFEDETPITNRSRPPHLRRATVFANGSLLLTQVRPRNAGIYRCIGQGQRGPPIILEATLHLAEIEDMPLFEPRVFTAGSEERVTCLPPKGLPEPSVWWEHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFKNGTLRINSVEVYDGTWYRCMSSTPAGSIEAQARVQVLEKLKFTPPPQPQQCMEFDKEATVPCSATGREKPTIKWERADGSSLPEWVTDNAGTLHFARVTRDDAGNYTCIASNGPQGQIRAHVQLTVAVFITFKVEPERTTVYQGHTALLQCEAQGDPKPLIQWKGKDRILDPTKLGPRMHIFQNGSLVIHDVAPEDSGRYTCIAGNSCNIKHTEAPLYVVDKPVPEESEGPGSPPPYKMIQTIGLSVGAAVAYIIAVLGLMFYCKKRCKAKRLQKQPEGEEPEMECLNGGPLQNGQPSAEIQEEVALTSLGSGPAATNKRHSTSDKMHFPRSSLQPITTLGKSEFGEVFLAKAQGLEEGVAETLVLVKSLQSKDEQQQLDFRRELEMFGKLNHANVVRLLGLCREAEPHYMVLEYVDLGDLKQFLRISKSKDEKLKSQPLSTKQKVALCTQVALGMEHLSNNRFVHKDLAARNCLVSAQRQVKVSALGLSKDVYNSEYYHFRQAWVPLRWMSPEAILEGDFSTKSDVWAFGVLMWEVFTHGEMPHGGQADDEVLADLQAGKARLPQPEGCPSKLYRLMQRCWALSPKDRPSFSEIASALGDSTVDSKP

SEQ ID NO: 76 - protein, 673 aa, Canis lupus
AIVFIKEPSSQDALQGRRALLRCEVEAPGPVHVHWLLNGAPVQDTERRFTQGSSLSFAAVDRLQDSGAFQCVARDDSTGEEARSANASFNIKWIETGPVVLKYPASEAEIQPQTQVTLRCHIDGHPRPTYQWFRDGSPLSDGQSNHTVSSKERNLTLRPASPEHSGLYSCCAQNAFGQTCSSHNFTLSIADESFARVVLAPQDLVVARNEEAMFHCQFSAQPPPSLQWVFEDDTPITNRSRPPHLRRATVFANGSLLLTQVRPRNAGIYRCIGQGQRGPPVVLEATLHLAEIEDMLPFEPRVFTAGSEERVACLPPQGLPEPSVWWEHAGARLPTHGRVFQEGHELVFAGTAESDAGVYTCHAANLAGQRRQDVNITVATVPTWLKKPQDSQLEEGKPGYLHCLTQATPKPTVIWYRNQMLISEDSRFEVSKNGTLRINSVEVYDGTWYRCVSSTPAGSIEAQARVQVLEKLKFTPPPRPQQCMEFDKEATVPCSATGREKPTIKWIRADGSSLPEWVTDNAGTLHFSRVTRDDAGNYTCIASNGLQGQIRAHVQLTVAVFITFKVEPERTTVYQGHTALLRCEAQGDPKPLIQWKGKDRILDPTKLGPRMHIFQNGSLVIHDVAPEDSGRYTCIAGNSCNIKHTEAPLYVVDKPVLEESEGPGSPPPYKMIQ

SEQ ID NO: 77 - protein, 119 aa, synthetic construct
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 78 - protein, 111 aa, synthetic construct
EIVLTQSPATLSLSPGERATLSCRASESVDSYGKSFMHWYQQKPGQAPRLLIYRASNLESGIPARFSGSGSGTDFTLTISSLEPEDFAVYYCQQSNEDPWTFGGGTKLEIK

SEQ ID NO: 79 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 80 - protein, 108 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 81 - protein, 121 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 82 - protein, 108 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKR

SEQ ID NO: 83 - protein, 107 aa, synthetic construct [sequence omitted]

SEQ ID NO: 84 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 86 - protein, 5 aa, synthetic construct
SYYWN

SEQ ID NO: 87 - protein, 16 aa, synthetic construct
YIYYSGSTNYNPSLKS

SEQ ID NO: 88 - protein, 12 aa, synthetic construct
DPHDYIRGHFDL

SEQ ID NO: 89 - protein, 7 aa, synthetic construct
GGSINSY

SEQ ID NO: 90 - protein, 5 aa, synthetic construct
YYSGS

SEQ ID NO: 91 - protein, 12 aa, synthetic construct
DPHDYIRGHFDL

SEQ ID NO: 92 - protein, 120 aa, synthetic construct
QVQLVQSGPGLVKPSETLSLTCTVSGGSINSYYWNWIRQPPGKGLEWIGYIYYSGSTNYNPSLKSRVTISVDTSKNQFSLKLSSVTAADTAVYYCARDPHDYIRGHFDLWGRGTLFTVSS

(19) United States
(12) Patent Application Publication
(10) Pub. No.: US 2026/0250421 A1
(43) Pub. Date: Aug. 27, 2026

(54) ANTIBODIES AND USES THEREOF

(71) Applicant: Biocytogen Pharmaceuticals (Beijing) Co., Ltd., Beijing (CN)

(72) Inventors: Sufei Yao, Beijing (CN); Liu Yang, Beijing (CN); Chengzhang Shang, Beijing (CN); Yi Yang, Beijing (CN); Yuelei Shen, Beijing (CN)

(21) Appl. No.: 19/163,585

(22) PCT Filed: Mar. 22, 2024
(86) PCT No.: PCT/CN2024/083178

§ 371 Date: Sep. 9, 2025

(30) Foreign Application Priority Data
Mar. 23, 2023 (WO) PCT/CN2023/083324
Aug. 21, 2023 (WO) PCT/CN2023/113943
Oct. 19, 2023 (WO) PCT/CN2023/125451

(51) Int. Cl. C07K 16/40; A61K 39/00; A61K 47/65; A61K 47/68; A61P 35/00; C07K 16/30

(52) U.S. Cl. CPC C07K 16/40; A61K 47/65; A61K 47/68031; A61K 47/68037; A61K 47/6843; A61P 35/00; C07K 16/30; A61K 2039/505; C07K 2317/31; C07K 2317/92

(57) ABSTRACT

The anti-PTK7 (protein tyrosine kinase 7) antibodies, antigen-binding fragments thereof, and antibody-drug conjugate (ADC) derived therefrom; anti-TROP2 (trophoblast cell surface antigen 2) antibodies, antigen-binding fragments thereof, and antibody-drug conjugate (ADC) derived therefrom; and anti-PTK7/TROP2 (e. g., multispecific or bispecific) antibodies or antigen-binding fragments thereof, and antibody-drug conjugate (ADC) derived therefrom and uses thereof are disclosed.

### CLAIM OF PRIORITY

This application claims priority to PCT/CN2023/083324, filed on Mar. 23, 2023; PCT/CN2023/113943, filed on Aug. 21, 2023; and PCT/CN2023/125451, filed on Oct. 19, 2023. The entire contents of the foregoing application are incorporated herein by reference.

### TECHNICAL FIELD

This disclosure relates to antibodies, antigen-binding fragments thereof, and antibody-drug conjugate (ADC) derived therefrom.

### BACKGROUND

Cancer is currently one of the diseases that have the highest human mortality. According to the World Health Organization statistical data, in 2012, the number of global cancer incidence and death cases reached 14 million and 8.2 million, respectively. In China, the newly diagnosed cancer cases are 3.07 million, and the death toll is 2.2 million. Recent clinical and commercial success of anticancer antibodies has created great interest in antibody-based therapeutics.

There is a need to develop antibodies for use in various antibody-based therapeutics to treat cancers.

### SUMMARY

This disclosure relates to anti-PTK7, anti-TROP2, and/or anti-PTK7/TROP2 antibodies, antigen-binding fragment thereof, and the uses thereof.

In one aspect, the disclosure is related to an antibody or antigen-binding fragment thereof that binds to PTK7 (protein tyrosine kinase 7) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 4-6, respectively, and the selected VL CDRs 1, 2, 3 amino acid sequences are set forth in SEQ ID NOs: 1-3,
(2) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 1-3,
(3) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 1-3, respectively;
(5) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 1-3, respectively;
(6) the selected VH CDRs 1, 2, 3 amino acid sequences are set forth in SEQ ID NOs: 19-21, respectively, and the selected VL CDRs 1, 2, 3 amino acid sequences are set forth in SEQ ID NOs: 1-3, respectively;
(7) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 1-3, respectively;
(8) the selected VH CDRs 1, 2, 3 amino acid sequences are set forth in SEQ ID NOs: 25-27, respectively, and the selected VL CDRs 1, 2, 3 amino acid sequences are set forth in SEQ ID NOs: 1-3, respectively;
(9) the selected VH CDRs 1, 2, 3 amino acid sequences are set forth in SEQ ID NOs: 28-30, respectively, and the selected VL CDRs 1, 2, 3 amino acid sequences are set forth in SEQ ID NOs: 1-3, respectively;
(10) the selected VH CDRs 1, 2, 3 amino acid sequences are set forth in SEQ ID NOs: 31-33, respectively, and the selected VL CDRs 1, 2, 3 amino acid sequences are set forth in SEQ ID NOs: 1-3, respectively;
(11) the selected VH CDRs 1, 2, 3 amino acid sequences are set forth in SEQ ID NOs: 34-36, respectively, and the selected VL CDRs 1, 2, 3 amino acid sequences are set forth in SEQ ID NOs: 1-3, respectively;
(12) the selected VH CDRs 1, 2, 3 amino acid sequences are set forth in SEQ ID NOs: 37-39, respectively, and the selected VL CDRs 1, 2, 3 amino acid sequences are set forth in SEQ ID NOs: 1-3, respectively;
(13) the selected VH CDRs 1, 2, 3 amino acid sequences are set forth in SEQ ID NOs: 40-42, respectively, and the selected VL CDRs 1, 2, 3 amino acid sequences are set forth in SEQ ID NOs: 1-3, respectively;
(14) the selected VH CDRs 1, 2, 3 amino acid sequences are set forth in SEQ ID NOs: 43-45, respectively, and the selected VL CDRs 1, 2, 3 amino acid sequences are set forth in SEQ ID NOs: 1-3, respectively;
(15) the selected VH CDRs 1, 2, 3 amino acid sequences are set forth in SEQ ID NOs: 86-88, respectively, and the selected VL CDRs 1, 2, 3 amino acid sequences are set forth in SEQ ID NOs: 1-3, respectively; and
(16) the selected VH CDRs 1, 2, 3 amino acid sequences are set forth in SEQ ID NOs: 89-91, respectively, and the selected VL CDRs 1, 2, 3 amino acid sequences are set forth in SEQ ID NOs: 1-3, respectively.

In some embodiments, the VH comprises CDRs 1, 2, 3 with the amino acid sequences set forth in SEQ ID NOs: 4-6, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 19-21, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 86-88, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 25-27, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 28-30, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 31-33, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 34-36, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 37-39, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 40-42, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 43-45, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 89-91, respectively, and the VL comprises CDRs 1, 2, 3 with the amino acid sequences set forth in SEQ ID NOs: 1-3, respectively, according to Chothia definition.

In some embodiments, the antibody or antigen-binding fragment thereof specifically binds to human, monkey, or dog PTK7.

In some embodiments, the antibody or antigen-binding fragment thereof is a human antibody or antigen-binding fragment thereof, a single-chain variable fragment (scFv), a one-armed antibody, and/or a multi-specific antibody (e.g., a bispecific antibody).

In some embodiments, the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

In one aspect, the disclosure relates to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 4-6, respectively, and wherein the VH, when paired with a light chain variable region (VL) comprising the amino acid sequence set forth in SEQ ID NO: 53 binds to PTK7;
(2) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 25-27, respectively, and wherein the VH, when paired with a light chain variable region (VL) comprising the amino acid sequence set forth in SEQ ID NO: 53 binds to PTK7;
(3) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 46 binds to PTK7;
(4) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7-9, respectively, and wherein the VH, when paired with a light chain variable region (VL) comprising the amino acid sequence set forth in SEQ ID NO: 53 binds to PTK7;
(5) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 28-30, respectively, and wherein the VH, when paired with a light chain variable region (VL) comprising the amino acid sequence set forth in SEQ ID NO: 53 binds to PTK7
(6) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 47 binds to PTK7;
(7) an immunoglobulin heavy chain or a fragment thereof comprising a VH comprising complementarity determining regions (CDRs) 1, 2, and 3 comprising the amino acid sequences set forth in SEQ ID NOs: 10-12, respectively, and wherein the VH, when paired with a light chain variable region (VL) comprising the amino acid sequence set forth in SEQ ID NO: 53 binds to PTK7;
(8) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 31-33, respectively, and wherein the VH, when paired with a light chain variable region (VL) comprising the amino acid sequence set forth in SEQ ID NO: 53 binds to PTK7;
(9) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 48 binds to PTK7;
(10) an immunoglobulin heavy chain or a fragment thereof comprising a VH comprising complementarity determining regions (CDRs) 1, 2, and 3 comprising the amino acid sequences set forth in SEQ ID NOs: 13-15, respectively, and wherein the VH, when paired with a light chain variable region (VL) comprising the amino acid sequence set forth in SEQ ID NO: 53 binds to PTK7;
(11) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 34-36, respectively, and wherein the VH, when paired with a light chain variable region (VL) comprising the amino acid sequence set forth in SEQ ID NO: 53 binds to PTK7;
(12) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 49 binds to PTK7;
(13) an immunoglobulin heavy chain or a fragment thereof comprising a VH comprising complementarity determining regions (CDRs) 1, 2, and 3 comprising the amino acid sequences set forth in SEQ ID NOs: 16-18, respectively, and wherein the VH, when paired with a light chain variable region (VL) comprising the amino acid sequence set forth in SEQ ID NO: 53 binds to PTK7;
(14) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 37-39, respectively, and wherein the VH, when paired with a light chain variable region (VL) comprising the amino acid sequence set forth in SEQ ID NO: 53 binds to PTK7;
(15) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 50 binds to PTK7;
(16) an immunoglobulin heavy chain or a fragment thereof comprising a VH comprising complementarity determining regions (CDRs) 1, 2, and 3 comprising the amino acid sequences set forth in SEQ ID NOs: 19-21, respectively, and wherein the VH, when paired with a light chain variable region (VL) comprising the amino acid sequence set forth in SEQ ID NO: 53 binds to PTK7;
(17) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 40-42, respectively, and wherein the VH, when paired with a light chain variable region (VL) comprising the amino acid sequence set forth in SEQ ID NO: 53 binds to PTK7;
(18) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 51 binds to PTK7;
(19) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 22-24, respectively, and wherein the VH, when paired with a light chain variable region (VL) comprising the amino acid sequence set forth in SEQ ID NO: 53 binds to PTK7;
(20) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 43-45, respectively, and wherein the VH, when paired with a light chain variable region (VL) comprising the amino acid sequence set forth in SEQ ID NO: 53 binds to PTK7;
(21) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 52 binds to PTK7.
(22) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 86-88, respectively, and wherein the VH, when paired with a light chain variable region (VL) comprising the amino acid sequence set forth in SEQ ID NO: 53 binds to PTK7;
(23) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 89-91, respectively, and wherein the VH, when paired with a light chain variable region (VL) comprising the amino acid sequence set forth in SEQ ID NO: 53 binds to PTK7; and
(24) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 92 binds to PTK7.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1-3, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 4-6, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7-9, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 10-12, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13-15, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 16-18, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 19-21, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 22-24, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 25-27, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 28-30, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 31-33, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 34-36, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 37-39, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 40-42, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 43-45, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 86-88, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 89-91, respectively.

In some embodiments, the VH when paired with a VL specifically binds to human, monkey, or dog PTK7, or the VL when paired with a VH specifically binds to human, monkey, or dog PTK7.

In some embodiments, the immunoglobulin heavy chain or the fragment thereof is a human immunoglobulin heavy chain or a fragment thereof (e.g., a human IgG1 heavy chain or a fragment thereof, a human IgG2 antibody or antigen-binding fragment thereof, or a human IgG4 heavy chain or a fragment thereof), and the immunoglobulin light chain or the fragment thereof is a human immunoglobulin light chain or a fragment thereof.

In some embodiments, the nucleic acid encodes a single-chain variable fragment (scFv), a one-armed antibody, a multi-specific antibody (e.g., a bispecific antibody), or a chimeric antigen receptor (CAR).

In some embodiments, the nucleic acid is cDNA.

In one aspect, the disclosure relates to a vector comprising one or more of the nucleic acids described herein.

In one aspect, the disclosure relates to a vector comprising two of the nucleic acids described herein, wherein the vector encodes the VL region and the VH region that together bind to PTK7.

In one aspect, the disclosure relates to a pair of vectors, wherein each vector comprises one of the nucleic acids described herein, wherein together the pair of vectors encodes the VL region and the VH region that together bind to PTK7.

In one aspect, the disclosure relates to a cell comprising the vector described herein, or the pair of vectors described herein.

In some embodiments, the cell is a CHO cell.

In one aspect, the disclosure relates to a cell comprising one or more of the nucleic acids described herein.

In one aspect, the disclosure relates to a cell comprising two of the nucleic acids described herein.

In some embodiments, the two nucleic acids together encode the VL region and the VH region that together bind to PTK7.

In one aspect, the disclosure relates to a method of producing an antibody or an antigen-binding fragment thereof, the method comprising
(a) culturing any one of the cells described herein under conditions sufficient for the cell to produce the antibody or the antigen-binding fragment; and
(b) collecting the antibody or the antigen-binding fragment produced by the cell.

In one aspect, the disclosure relates to an antibody or antigen-binding fragment thereof that binds to PTK7 comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 46, and the selected VL sequence is SEQ ID NO: 53;
(2) the selected VH sequence is SEQ ID NO: 47, and the selected VL sequence is SEQ ID NO: 53;
(3) the selected VH sequence is SEQ ID NO: 48, and the selected VL sequence is SEQ ID NO: 53;
(4) the selected VH sequence is SEQ ID NO: 49, and the selected VL sequence is SEQ ID NO: 53;
(5) the selected VH sequence is SEQ ID NO: 50, and the selected VL sequence is SEQ ID NO: 53;
(6) the selected VH sequence is SEQ ID NO: 51, and the selected VL sequence is SEQ ID NO: 53;
(7) the selected VH sequence is SEQ ID NO: 52, and the selected VL sequence is SEQ ID NO: 53; and
(8) the selected VH sequence is SEQ ID NO: 92, and the selected VL sequence is SEQ ID NO: 53.

In some embodiments, the VH comprises the sequence of SEQ ID NO: 46 and the VL comprises the sequence of SEQ ID NO: 53.

In some embodiments, the VH comprises the sequence of SEQ ID NO: 47 and the VL comprises the sequence of SEQ ID NO: 53.

In some embodiments, the VH comprises the sequence of SEQ ID NO: 48 and the VL comprises the sequence of SEQ ID NO: 53.

In some embodiments, the VH comprises the sequence of SEQ ID NO: 49 and the VL comprises the sequence of SEQ ID NO: 53.

In some embodiments, the VH comprises the sequence of SEQ ID NO: 50 and the VL comprises the sequence of SEQ ID NO: 53.

In some embodiments, the VH comprises the sequence of SEQ ID NO: 51 and the VL comprises the sequence of SEQ ID NO: 53.

In some embodiments, the VH comprises the sequence of SEQ ID NO: 52 and the VL comprises the sequence of SEQ ID NO: 53.

In some embodiments, the VH comprises the sequence of SEQ ID NO: 92 and the VL comprises the sequence of SEQ ID NO: 53.

In one aspect, the disclosure relates to an antibody or antigen-binding fragment thereof that binds to PTK7 comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
   (1) the selected VH sequence is SEQ ID NO: 46, and the selected VL sequence is SEQ ID NO: 53;
   (2) the selected VH sequence is SEQ ID NO: 47, and the selected VL sequence is SEQ ID NO: 53;
   (3) the selected VH sequence is SEQ ID NO: 48, and the selected VL sequence is SEQ ID NO: 53;
   (4) the selected VH sequence is SEQ ID NO: 49, and the selected VL sequence is SEQ ID NO: 53;
   (5) the selected VH sequence is SEQ ID NO: 50, and the selected VL sequence is SEQ ID NO: 53;
   (6) the selected VH sequence is SEQ ID NO: 51, and the selected VL sequence is SEQ ID NO: 53;
   (7) the selected VH sequence is SEQ ID NO: 52, and the selected VL sequence is SEQ ID NO: 53; and
   (8) the selected VH sequence is SEQ ID NO: 92, and the selected VL sequence is SEQ ID NO: 53.

In some embodiments, the antibody or antigen-binding fragment thereof specifically binds to human, monkey, or dog PTK7.

In some embodiments, the antibody or antigen-binding fragment thereof is a human antibody or antigen-binding fragment thereof, a single-chain variable fragment (scFv), a one-armed antibody, and/or a multi-specific antibody (e.g., a bispecific antibody).

In some embodiments, the antibody or antigen-binding fragment is a human IgG1 antibody or antigen-binding fragment thereof, a human IgG2 antibody or antigen-binding fragment thereof, or a human IgG4 antibody or antigen-binding fragment thereof.

In one aspect, the disclosure relates to an antibody or antigen-binding fragment thereof that binds to TROP2 (trophoblast cell surface antigen 2) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 57-59, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 60-62, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 66-68, respectively, and the selected VL CDRs 1, 2, 3 amino acid sequences are set forth in SEQ ID NOs: 1-3, respectively; and
(4) the selected VH CDRs 1, 2, 3 amino acid sequences are set forth in SEQ ID NOs: 69-71, respectively, and the selected VL CDRs 1, 2, 3 amino acid sequences are set forth in SEQ ID NOs: 1-3, respectively.

In some embodiments, the VH comprises CDRs 1, 2, 3 with the amino acid sequences set forth in SEQ ID NOs: 57-59, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 60-62, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 66-68, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 69-71, respectively, and the VL comprises CDRs 1, 2, 3 with the amino acid sequences set forth in SEQ ID NOs: 1-3, respectively, according to Chothia definition.

In some embodiments, the antibody or antigen-binding fragment thereof specifically binds to human, monkey, or dog TROP2.

In some embodiments, the antibody or antigen-binding fragment thereof is a human antibody or antigen-binding fragment thereof, a single-chain variable fragment (scFv), a one-armed antibody, and/or a multi-specific antibody (e.g., a bispecific antibody).

In some embodiments, the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

In one aspect, the disclosure relates to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 57-59, respectively, and wherein the VH, when paired with a light chain variable region (VL) comprising the amino acid sequence set forth in SEQ ID NO: 53 binds to TROP2;
(2) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 66-68, respectively, and wherein the VH, when paired with a light chain variable region (VL) comprising the amino acid sequence set forth in SEQ ID NO: 53 binds to TROP2;
(3) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 73 binds to TROP2;
(4) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 60-62, respectively, and wherein the VH, when paired with a light chain variable region (VL) comprising the amino acid sequence set forth in SEQ ID NO: 53 binds to TROP2;
(5) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 69-71, respectively, and wherein the VH, when paired with a light chain variable region (VL) comprising the amino acid sequence set forth in SEQ ID NO: 53 binds to TROP2; and
(6) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 74 binds to TROP2.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1-3, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 57-59, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 60-62, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 66-68, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 69-71, respectively.

In some embodiments, the VH when paired with a VL specifically binds to human, monkey, or dog TROP2, or the VL when paired with a VH specifically binds to human, monkey, or dog TROP2.

In some embodiments, the immunoglobulin heavy chain or the fragment thereof is a human immunoglobulin heavy chain or a fragment thereof (e.g., a human IgG1 heavy chain or a fragment thereof, a human IgG2 antibody or antigen-binding fragment thereof, or a human IgG4 heavy chain or a fragment thereof), and the immunoglobulin light chain or the fragment thereof is a human immunoglobulin light chain or a fragment thereof.

In some embodiments, the nucleic acid encodes a single-chain variable fragment (scFv), a one-armed antibody, a multi-specific antibody (e.g., a bispecific antibody), or a chimeric antigen receptor (CAR).

In some embodiments, the nucleic acid is cDNA.

In one aspect, the disclosure relates to a vector comprising one or more of the nucleic acids described herein.

In one aspect, the disclosure relates to a vector comprising two of the nucleic acids described herein, wherein the vector encodes the VL region and the VH region that together bind to TROP2.

In one aspect, the disclosure relates to a pair of vectors, wherein each vector comprises one of the nucleic acids described herein, wherein together the pair of vectors encodes the VL region and the VH region that together bind to TROP2.

In one aspect, the disclosure relates to a cell comprising the vector described herein, or the pair of vectors described herein.

In some embodiments, the cell is a CHO cell.

In one aspect, the disclosure relates to a cell comprising one or more of the nucleic acids described herein.

In one aspect, the disclosure relates to a cell comprising two of the nucleic acids described herein.

In some embodiments, the two nucleic acids together encode the VL region and the VH region that together bind to TROP2.

In one aspect, the disclosure relates to a method of producing an antibody or an antigen-binding fragment thereof, the method comprising
(c) culturing any of the cells described herein under conditions sufficient for the cell to produce the antibody or the antigen-binding fragment; and
(d) collecting the antibody or the antigen-binding fragment produced by the cell.

In one aspect, the disclosure relates to an antibody or antigen-binding fragment thereof that binds to TROP2 comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 73, and the selected VL sequence is SEQ ID NO: 53; and
(2) the selected VH sequence is SEQ ID NO: 74, and the selected VL sequence is SEQ ID NO: 53.

In some embodiments, the VH comprises the sequence of SEQ ID NO: 73 and the VL comprises the sequence of SEQ ID NO: 53.

In some embodiments, the VH comprises the sequence of SEQ ID NO: 74 and the VL comprises the sequence of SEQ ID NO: 53.

In one aspect, the disclosure relates to an antibody or antigen-binding fragment thereof that binds to TROP2 comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
   (1) the selected VH sequence is SEQ ID NO: 73, and the selected VL sequence is SEQ ID NO: 53; and
   (2) the selected VH sequence is SEQ ID NO: 74, and the selected VL sequence is SEQ ID NO: 53.

In some embodiments, the antibody or antigen-binding fragment thereof specifically binds to human, monkey, or dog TROP2.

In some embodiments, the antibody or antigen-binding fragment thereof is a human antibody or antigen-binding fragment thereof, a single-chain variable fragment (scFv), a one-armed antibody, and/or a multi-specific antibody (e.g., a bispecific antibody).

In some embodiments, the antibody or antigen-binding fragment is a human IgG1 antibody or antigen-binding fragment thereof, a human IgG2 antibody or antigen-binding fragment thereof, or a human IgG4 antibody or antigen-binding fragment thereof.

In one aspect, the disclosure relates to an anti-PTK7/TROP2 antibody (e.g., bispecific antibody) or antigen-binding fragment thereof, comprising: a first antigen-binding domain that specifically binds to an epitope of PTK7; and a second antigen-binding domain that specifically binds to an epitope of TROP2.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3,
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively;
(10) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively;
(11) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively;
(12) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively;
(13) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively;
(14) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3,
(15) the selected VH1 CDRs 1, 2, 3 amino acid sequences are set forth in SEQ ID NOs: 86-88, respectively, and the selected VL1 CDRs 1, 2, 3 amino acid sequences are set forth in SEQ ID NOs: 1-3, respectively; and
(16) the selected VH1 CDRs 1, 2, 3 amino acid sequences are set forth in SEQ ID NOs: 89-91, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively.

In some embodiments, selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 86-88, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 89-91, respectively, and the selected VL1 CDRs 1, 2, 3 amino acid sequences are set forth in SEQ ID NOs: 1-3, respectively.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(4) the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(5) the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively; and
(6) the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively.

In some embodiments, the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively.

In some embodiments, the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively.

In some embodiments, the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively.

In some embodiments, the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments:
(1) The selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(2) The selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(3) The selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(4) The selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(5) The selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(6) The selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOS: 1-3, respectively;
(7) The selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(8) The selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(9) The selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOS: 1-3, respectively;
(10) The selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(11) The selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(12) The selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(13) The selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(14) The selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(15) The selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(16) The selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(17) The selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(18) The selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(19) The selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(20) The selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(21) The selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOS: 1-3, respectively;
(22) The selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(23) The selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOS: 1-3, respectively;
(24) The selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(25) The selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOS: 1-3, respectively;
(26) The selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(27) The selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOS: 1-3, respectively;
(28) The selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(29) The selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOS: 1-3, respectively;
(30) The selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOS: 1-3, respectively;
(31) The selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively;
(32) The selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOS: 1-3, respectively;
(33) The selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOS: 1-3, respectively;
(34) The selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively;
(35) The selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOS: 1-3, respectively;
(36) The selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(37) The selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(38) The selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(39) The selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(40) The selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(41) The selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(42) The selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(43) The selected VH1 CDRs 1, 2, 3 amino acid sequences are set forth in SEQ ID NOs: 86-88, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(44) The selected VH1 CDRs 1, 2, 3 amino acid sequences are set forth in SEQ ID NOs: 89-91, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(45) The selected VH1 CDRs 1, 2, 3 amino acid sequences are set forth in SEQ ID NOs: 86-88, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively; and
(46) The selected VH1 CDRs 1, 2, 3 amino acid sequences are set forth in SEQ ID NOs: 89-91, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively.

In some embodiments, the first heavy chain variable region comprises a sequence that is at least 80%, 85%, 90%, 95%, 99%, or 100% identical to SEQ ID NO: 46, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 52, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 46, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 52, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 46, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 52, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 92, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 92, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the VH1 comprises an amino acid sequence that is at least 80%, 85%, 90%, 95%, 99%, or 100% identical to a selected VH sequence, and the VL1 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 46, and the selected VL sequence is SEQ ID NO: 53;
(2) the selected VH sequence is SEQ ID NO: 47, and the selected VL sequence is SEQ ID NO: 53;
(3) the selected VH sequence is SEQ ID NO: 48, and the selected VL sequence is SEQ ID NO: 53;
(4) the selected VH sequence is SEQ ID NO: 49, and the selected VL sequence is SEQ ID NO: 53;
(5) the selected VH sequence is SEQ ID NO: 50, and the selected VL sequence is SEQ ID NO: 53;
(6) the selected VH sequence is SEQ ID NO: 51, and the selected VL sequence is SEQ ID NO: 53;
(7) the selected VH sequence is SEQ ID NO: 52, and the selected VL sequence is SEQ ID NO: 53; and
(8) the selected VH sequence is SEQ ID NO: 92, and the selected VL sequence is SEQ ID NO: 53.

In some embodiments, the VH2 comprises an amino acid sequence that is at least 80%, 85%, 90%, 95%, 99%, or 100% identical to a selected VH sequence, and the VL2 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 72, and the selected VL sequence is SEQ ID NO: 53;
(2) the selected VH sequence is SEQ ID NO: 73, and the selected VL sequence is SEQ ID NO: 53; and
(3) the selected VH sequence is SEQ ID NO: 74, and the selected VL sequence is SEQ ID NO: 53.

In some embodiments, the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 46, and the selected VL sequence is SEQ ID NO: 53;
(2) the selected VH sequence is SEQ ID NO: 47, and the selected VL sequence is SEQ ID NO: 53;
(3) the selected VH sequence is SEQ ID NO: 48, and the selected VL sequence is SEQ ID NO: 53;
(4) the selected VH sequence is SEQ ID NO: 49, and the selected VL sequence is SEQ ID NO: 53;
(5) the selected VH sequence is SEQ ID NO: 50, and the selected VL sequence is SEQ ID NO: 53;
(6) the selected VH sequence is SEQ ID NO: 51, and the selected VL sequence is SEQ ID NO: 53;
(7) the selected VH sequence is SEQ ID NO: 52, and the selected VL sequence is SEQ ID NO: 53; and
(8) the selected VH sequence is SEQ ID NO: 92, and the selected VL sequence is SEQ ID NO: 53.

In some embodiments, the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 72, and the selected VL sequence is SEQ ID NO: 53;
(2) the selected VH sequence is SEQ ID NO: 73, and the selected VL sequence is SEQ ID NO: 53; and
(3) the selected VH sequence is SEQ ID NO: 74, and the selected VL sequence is SEQ ID NO: 53.

In some embodiments, the first antigen-binding domain specifically binds to human, monkey, or dog PTK7; and/or the second antigen-binding domain specifically binds to human, monkey, or dog TROP2.

In some embodiments, the first antigen-binding domain is a human or humanized antigen-binding domain; and/or the second antigen-binding domain is a human or humanized antigen-binding domain.

In some embodiments, the first antigen-binding domain is a single-chain variable fragment (scFv); and/or the second antigen-binding domain is a scFv.

In some embodiments, the first light chain variable region and the second light chain variable region are identical.

In one aspect, the disclosure relates to an antibody or antigen-binding fragment thereof that cross-competes with any one of the antibodies or antigen-binding fragments thereof described herein.

In some embodiments, the antibody or antigen-binding fragment thereof comprises a fragment crystallizable region (Fc region).

In some embodiments, the Fc region has increased complement-dependent cytotoxicity (CDC) or antibody-dependent cellular cytotoxicity (ADCC).

In one aspect, the disclosure relates to a chimeric antigen receptor (CAR) comprising any one of the antibodies or antigen-binding fragments thereof described herein.

In one aspect, the disclosure relates to an antibody-drug conjugate comprising any one of the antibodies or antigen-binding fragments thereof described herein covalently bound to a therapeutic agent.

In some embodiments, the therapeutic agent is a cytotoxic or cytostatic agent.

In some embodiments, the therapeutic agent is MMAE or MMAF.

In some embodiments, the therapeutic agent is selected from

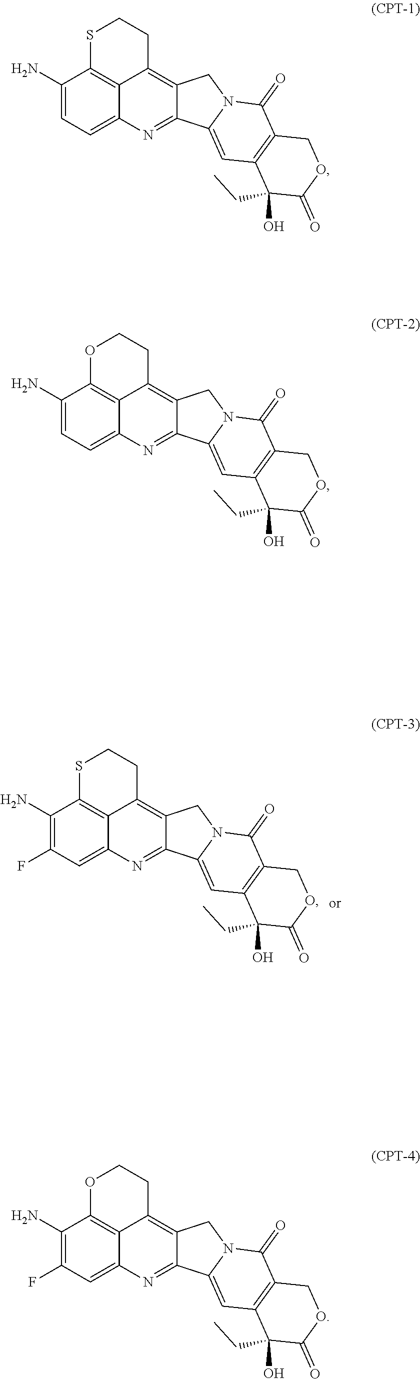 [SMILES: CC[C@@]1(O)C(=O)OCc2c1cc1n(c2=O)Cc2c-1nc1ccc(N)c3c1c2CCS3 ; CC[C@@]1(O)C(=O)OCc2c1cc1n(c2=O)Cc2c-1nc1ccc(N)c3c1c2CCO3 ; CC[C@@]1(O)C(=O)OCc2c1cc1n(c2=O)Cc2c-1nc1cc(F)c(N)c3c1c2CCS3 ; CC[C@@]1(O)C(=O)OCc2c1cc1n(c2=O)Cc2c-1nc1cc(F)c(N)c3c1c2CCO3]

In some embodiments, the therapeutic agent is linked to the antibody or antigen-binding fragment thereof via a linker. In some embodiments, the linker has a structure of:

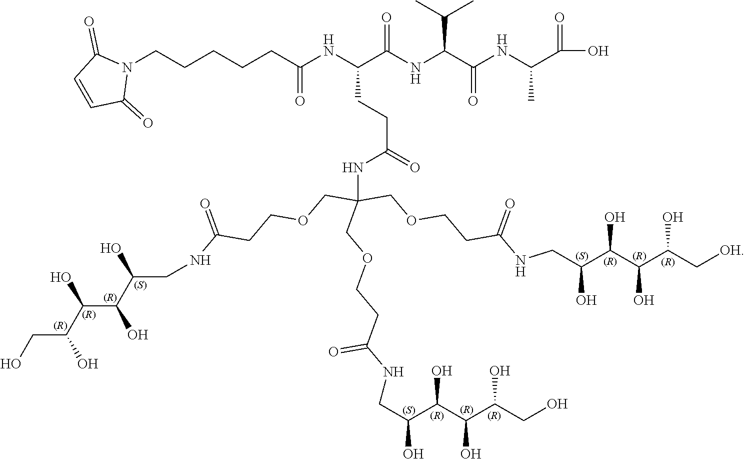

In some embodiments, the antibody-drug conjugate has a structure of:

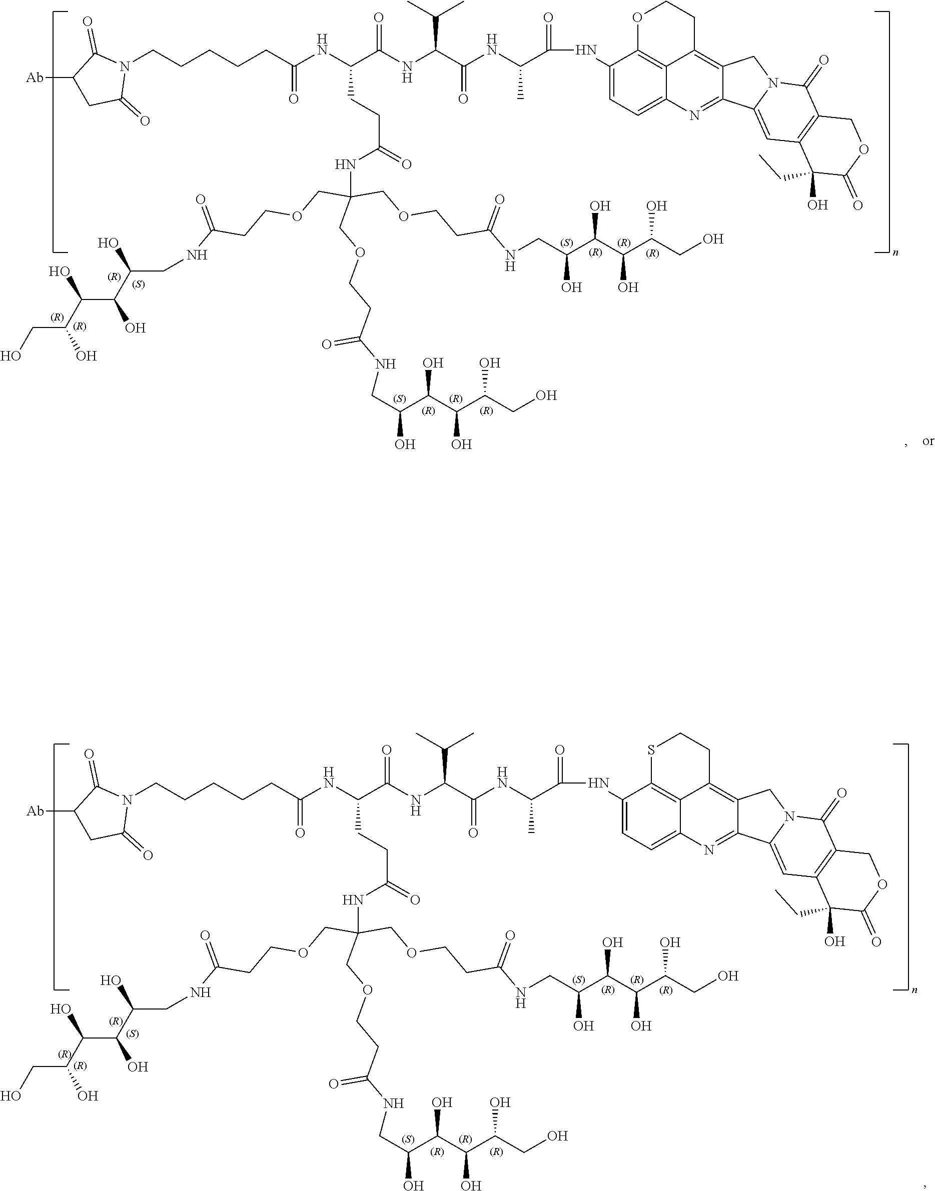

in some embodiments, n=1, 2, 3, 4, 5, 6, 7, or 8; in some embodiments, “Ab” represents the antibody or antigen-binding fragment thereof described herein.

In one aspect, the disclosure relates to a method of treating a subject having cancer, the method comprising administering a therapeutically effective amount of a composition comprising any one of the antibodies or antigen-binding fragments thereof described herein, any one of the CARs described herein, or any one of the antibody-drug conjugates described herein.

In some embodiments, the subject has a solid tumor.

In some embodiments, the cancer is colon cancer, triple-negative breast cancer (TNBC), lung cancer, non-small cell lung cancer (NSCLC), esophageal cancer, pancreatic cancer, colorectal cancer, ovarian cancer (OVCA), or bladder cancer.

In some embodiments, the subject is further treated with an effective amount of an anti-4-1BB antibody, an anti-OX40 antibody, an anti-PD-1 antibody, an anti-CTLA4 antibody, an anti-CD40 antibody, or an anti-PD-L1 antibody.

In one aspect, the disclosure relates to a method of decreasing the rate of tumor growth, the method comprising contacting a tumor cell with an effective amount of a composition comprising any one of the antibodies or antigen-binding fragments thereof described herein, any one of the CARs described herein, or any one of the antibody-drug conjugates described herein.

In one aspect, the disclosure relates to a method of killing a tumor cell, the method comprising contacting a tumor cell with an effective amount of a composition comprising any one of the antibodies or antigen-binding fragments thereof described herein, any one of the CARs described herein, or any one of the antibody-drug conjugates described herein.

In one aspect, the disclosure relates to a method of increasing immune response in a subject, the method comprising administering to the subject an effective amount of a composition comprising any one of the antibodies or antigen-binding fragments thereof described herein, any one of the CARs described herein, or any one of the antibody-drug conjugates described herein.

In one aspect, the disclosure relates to a pharmaceutical composition comprising any one of the antibodies or antigen-binding fragments thereof described herein, and a pharmaceutically acceptable carrier.

In one aspect, the disclosure relates to a pharmaceutical composition comprising any one of the antibody drug conjugates described herein, and a pharmaceutically acceptable carrier.

In some embodiments, the drug-to-antibody ratio (DAR) is about 4 or 8.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “human antibody” refers to an antibody that is encoded by an endogenous nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the term “multimeric antibody” refers to an antibody that contains four or more (e.g., six, eight, or ten) immunoglobulin variable domains.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., PTK7) preferably to some other molecules in general, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a PTK7 molecule may be referred to as a PTK7-specific antibody or an anti-PTK7 antibody. Similarly, an antibody that specifically binds to both PTK7 and TROP2 molecules may be referred to as an anti-PTK7/TROP2 antibody.

As used herein, the term “bispecific antibody” refers to an antibody that binds to two different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, or six different epitopes.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A lists Kabat CDR sequences for anti-PTK7 antibodies.

FIG. 1B lists Kabat CDR sequences for anti-TROP2 antibodies.

FIG. 2A lists Chothia CDR sequences for anti-PTK7 antibodies.

FIG. 2B lists Chothia CDR sequences for anti-TROP2 antibodies.

FIG. 3 lists sequences of the heavy chain variable regions and light chain variable regions of the anti-PTK7 and anti-TROP2 antibodies a antigen-binding fragments thereof.

FIG. 4 lists some of the amino acids sequences discussed in the disclosure.

FIG. 5 shows the tumor volume of mice xenograft with MDA-MB-468 cells in different groups treated with the ADCs described herein. Sacituzumab govitecan and hu24-ADC were used as positive controls.

FIG. 6 shows the tumor volume of mice xenograft with patient-derived breast tumor fragments in different groups treated with the ADCs described herein.

FIG. 7 shows the tumor volume of mice xenograft with patient-derived lung tumor fragments in different groups treated with the ADCs described herein.

FIG. 8 shows the tumor volume of mice xenograft with patient-derived pancreatic tumor fragments in different groups treated with the ADCs described herein.

FIG. 9 shows the tumor volume of mice xenograft with patient-derived colorectal tumor fragments in different groups treated with the ADCs described herein.

FIG. 10 shows the tumor volume of mice xenograft with patient-derived colorectal tumor fragments in different groups treated with the ADCs described herein.

FIG. 11 shows the binding activity results of antibodies in NCI-H1975 cells (A) and NUGC-4 cells (B).

FIG. 12 shows the in vitro killing activity of ADCs in NCI-H1975 cells (A) and NUGC-4 cells (B).

### DETAILED DESCRIPTION

A bispecific antibody or antigen-binding fragment thereof is an artificial protein that can simultaneously bind to two different epitopes (e.g., on two different antigens). In some embodiments, a bispecific antibody or antigen-binding fragment thereof can have two arms. Each arm can have one heavy chain variable region and one light chain variable region, forming an antigen-binding domain (or an antigen-binding region). In some embodiments, the bispecific antibody has a common light chain.

The present disclosure provides examples of antibodies, antigen-binding fragment thereof, that bind to PTK7, TROP2, and/or both PTK7 and TROP2 (PTK7/TROP2), antibody drug conjugates derived from these antibodies. The present disclosure also relates to anti-PTK7/TROP2 antibodies (e.g., bispecific antibodies or antigen-binding fragments thereof) that specifically bind to PTK7 and TROP, and antibody drug conjugates derived from these anti-PTK7/TROP2 antibodies.

### PTK7 and TROP2

Protein tyrosine kinase 7 (PTK7) is a highly conserved member of the pseudokinase family of receptor tyrosine kinases. The lack of observable kinase activity across species is likely explained by substitutions at residues that are typically conserved in kinase domains. Genetic and biochemical studies have demonstrated a key function for PTK7 in noncanonical Wnt signaling, and PTK7-deficient embryos exhibit severe developmental defects in planar cell polarity. There is also evidence for additional, possibly context-dependent functions of PTK7 in the vascular endothelial growth factor (VEGF), semaphorin/plexin, and canonical Wnt signaling pathways. Oncogenic functions of PTK7 have been documented in colon cancer, lung cancer, and esophageal cancer, and PTK7 promotes cell survival and resistance to chemotherapy in acute myeloid leukemia.

PTK7 is highly expressed in breast cancer and correlates with worse prognosis and associates with tumor metastasis and progression in TNBC. Co-expression analysis and gain- or loss-of-function of PTK7 in TNBC cell lines revealed that PTK7 participates in EGFR/Akt signaling regulation and associated with extracellular matrix organization and migration genes in breast cancer, including COL1A1, FN1, WNT5B, MMP11, MMP14 and SDC1. Gain- or loss-of-function experiments of PTK7 suggested that PTK7 promotes proliferation and migration in TNBC cell lines. PTK7 knockdown cell bearing mouse model further demonstrated that PTK7-deficiency inhibits TNBC tumor progression in vivo.

A detailed review of PTK7 and its functions can be found in Damelin M, et al., A PTK7-targeted antibody-drug conjugate reduces tumor-initiating cells and induces sustained tumor regressions. Sci Transl Med. 2017 Jan. 11; 9(372):eaag2611. doi: 10.1126/scitranslmed.aag2611. PMID: 28077676; Xiang et al., Biomater Res. 2022 Dec. 5; 26(1):74. doi: 10.1186/s40824-022-00328-9; Cui et al., Front. Oncol., 22 Jul. 2021, Sec. Breast Cancer, Volume 11-2021; and Kim et al., Int J Mol Sci. 2022 Oct. 13; 23(20):12195. doi: 10.3390/ijms232012195, each of which is incorporated by reference in its entirety.

Trophoblast cell surface antigen 2 (TROP2) is a glycoprotein that spans the epithelial membrane surface and plays a role in cell self-renewal, proliferation, and transformation. Encoded by the TACSTD2 gene, TROP2 is a 35-kDa protein composed of a large extracellular domain, a single transmembrane domain, and a short intracellular tail that is the functionally dominant part of the protein.

Under physiological conditions, TROP2 plays an essential role in embryonic development, placental tissue formation, embryo implantation, stem cell proliferation, and organ development. A low basal expression level of TROP2 is found on the surface of multiple normal epithelial tissues, including skin and oral mucosa. TROP2 can promote tumor growth and its overexpression is common in many types of malignant epithelial tumors.

Expression of Trop2 is regulated by several pro-oncogenic transcription factors (e.g., CREB1, nuclear factor NFκB, and HOXA10) via positive feedback relationships. Trop2 expression may be upregulated because of the inactivation of several transcription factors (e.g., HNF4A, TP63/TP53L, ERG, HNF1A/TCF-1, and FOXP3). Overexpression of TROP2 accelerates the cancer cell cycle and drives cancer growth. Knocking out the TACSTD2 gene disturbs the proliferation of tumor cells, further validating the role of TROP2 in tumorigenesis.

TROP2 was elucidated as a transducer of intracellular calcium signals; and it is known to function in a variety of cell signaling pathways associated with tumorigenesis. Expression of TROP2, as a calcium signal transducer, causes calcium to be mobilized from internal stores. Increased intracellular calcium levels activate MAPK, which in turn increases levels of phosphorylated ERK1 and ERK2. ERK1 and ERK2 are important mediators of cell cycle progression, angiogenesis, cell proliferation, cell invasion, and metastasis. Intracellular calcium also activates the NFκB pathway, which is involved in stimulation of cell growth, and the RAF pathway, which is essential for the upregulation of FOXM1, one of the most commonly overexpressed genes in human solid tumors.

In addition to stimulating calcium release and MAPK signaling, TROP2 is involved in several other pro-oncogenic signaling pathways, leading to tumor cell growth and proliferation. Activation of cyclin E and D further promotes cell cycle progression. Alteration of the Notch, Hedgehog, and Wnt pathways may discourage appropriate stem cell proliferation and differentiation. TROP2 signaling was also shown to be dependent on β-catenin. Direct interaction between β-catenin and the intracellular domain of TROP2, through β-catenin signaling, enhances stem cell-like properties (e.g., self-renewal and transformation) of cancer cells. Attenuation of IGF-1 receptor signaling by TROP2 encourages cancer growth and malignancy, particularly in lung cancers.

A detailed review of TROP2 and its functions can be found in Zaman S, Jadid H, Denson A C, Gray J E. Targeting Trop-2 in solid tumors: future prospects. Onco Targets Ther. 2019; 12:1781-1790. doi: 10.2147/OTT.S162447; Shvartsur A, Bonavida B. Trop2 and its overexpression in cancers: regulation and clinical/therapeutic implications. Genes Cancer. 2015; 6(3-4):84-105. doi: 10.18632/genesandcancer.40; Strop P, Tran T T, Dorywalska M, et al. RN927C, a site-specific Trop-2 antibody-drug conjugate (ADC) with enhanced stability, is highly efficacious in preclinical solid tumor models. Mol Cancer Ther. 2016; 15(11):2698-2708. doi: 10.1158/1535-7163.MCT-16-0431; and Goldenberg D M, Stein R, Sharkey R M. The emergence of trophoblast cell-surface antigen 2 (TROP-2) as a novel cancer target. Oncotarget. 2018; 9(48):28989-29006. doi: 10.18632/oncotarget.25615; each of which is incorporated by reference in its entirety.

The present disclosure provides several anti-PTK7, anti-TROP2, and anti-PTK7/TROP2 (e.g., multispecific or bispecific) antibodies, antigen-binding fragments thereof, and methods of using these anti-PTK7, anti-TROP2, and/or anti-PTK7/TROP2 antibodies and antigen-binding fragments to inhibit tumor growth, treat cancers, and to treat autoimmune diseases.

### Anti-PTK7 Antibodies and Antigen-Binding Fragments

The disclosure provides antibodies and antigen-binding fragments thereof that specifically bind to PTK7 (e.g., human PTK7, monkey PTK7 or dog PTK7). The antibodies and antigen-binding fragments described herein are capable of binding to PTK7. These antibodies can be agonists or antagonists. In some embodiments, these antibodies can increase immune response. In some embodiments, these antibodies can block PTK7 activity, e.g., reduce the frequency of tumor-initiating cells (TICs) or inhibit angiogenesis and the stimulation of immune cells.

The disclosure provides e.g., anti-PTK7 antibodies 1A7, 1A11, 1F2, 2A5, 2D11, 2F5, 3C4, 3E6, the chimeric antibodies thereof, and the human or humanized antibodies thereof.

The CDR sequences for 1A7, and 1A7 derived antibodies (e.g., human or humanized antibodies) include CDRs of the heavy chain variable domain, SEQ ID NOs: 4, 5, 6 and CDRs of the light chain variable domain, SEQ ID NOs: 1, 2, 3 as defined by Kabat numbering. The CDRs can also be defined by Chothia system. Under the Chothia numbering, the CDR sequences of the heavy chain variable domain are set forth in SEQ ID NOs: 25, 26, 27 and CDR sequences of the light chain variable domain are set forth in SEQ ID NOs: 1, 2, 3.

The CDR sequences for 1A11, and 1A11 derived antibodies (e.g., human or humanized antibodies) include CDRs of the heavy chain variable domain, SEQ ID NOs: 86, 87, 88 and CDRs of the light chain variable domain, SEQ ID NOs: 1, 2, 3 as defined by Kabat numbering. The CDRs can also be defined by Chothia system. Under the Chothia numbering, the CDR sequences of the heavy chain variable domain are set forth in SEQ ID NOs: 89, 90, 91 and CDR sequences of the light chain variable domain are set forth in SEQ ID NOs: 1, 2, 3.

The CDR sequences for 1F2, and 1F2 derived antibodies (e.g., human or humanized antibodies) include CDRs of the heavy chain variable domain, SEQ ID NOs: 7, 8, 9, and CDRs of the light chain variable domain, SEQ ID NOs: 1, 2, 3 as defined by Kabat numbering. The CDRs can also be defined by Chothia system. Under the Chothia numbering, the CDR sequences of the heavy chain variable domain are set forth in SEQ ID NOs: 28, 29, 30 and CDR sequences of the light chain variable domain are set forth in SEQ ID NOs: 1, 2, 3.

The CDR sequences for 2A5, and 2A5 derived antibodies (e.g., human or humanized antibodies) include CDRs of the heavy chain variable domain, SEQ ID NOs: 10, 11, 12, and CDRs of the light chain variable domain, SEQ ID NOs: 1, 2, 3 as defined by Kabat numbering. The CDRs can also be defined by Chothia system. Under the Chothia numbering, the CDR sequences of the heavy chain variable domain are set forth in SEQ ID NOs: 31, 32, 33 and CDR sequences of the light chain variable domain are set forth in SEQ ID NOs: 1, 2, 3.

The CDR sequences for 2D11, and 2D11 derived antibodies (e.g., human or humanized antibodies) include CDRs of the heavy chain variable domain, SEQ ID NOs: 13, 14, 15, and CDRs of the light chain variable domain, SEQ ID NOs: 1, 2, 3 as defined by Kabat numbering. The CDRs can also be defined by Chothia system. Under the Chothia numbering, the CDR sequences of the heavy chain variable domain are set forth in SEQ ID NOs: 34, 35, 36 and CDR sequences of the light chain variable domain are set forth in SEQ ID NOs: 1, 2, 3.

The CDR sequences for 2F5, and 2F5 derived antibodies (e.g., human or humanized antibodies) include CDRs of the heavy chain variable domain, SEQ ID NOs: 16, 17, 18, and CDRs of the light chain variable domain, SEQ ID NOs: 1, 2, 3 as defined by Kabat numbering. The CDRs can also be defined by Chothia system. Under the Chothia numbering, the CDR sequences of the heavy chain variable domain are set forth in SEQ ID NOs: 37, 38, 39 and CDR sequences of the light chain variable domain are set forth in SEQ ID NOs: 1, 2, 3.

The CDR sequences for 3C4, and 3C4 derived antibodies (e.g., human or humanized antibodies) include CDRs of the heavy chain variable domain, SEQ ID NOs: 19, 20, 21, and CDRs of the light chain variable domain, SEQ ID NOs: 1, 2, 3 as defined by Kabat numbering. The CDRs can also be defined by Chothia system. Under the Chothia numbering, the CDR sequences of the heavy chain variable domain are set forth in SEQ ID NOs: 40, 41, 42 and CDR sequences of the light chain variable domain are set forth in SEQ ID NOs: 1, 2, 3.

The CDR sequences for 3E6, and 3E6 derived antibodies (e.g., human or humanized antibodies) include CDRs of the heavy chain variable domain, SEQ ID NOs: 22, 23, 24, and CDRs of the light chain variable domain, SEQ ID NOs: 1, 2, 3 as defined by Kabat numbering. The CDRs can also be defined by Chothia system. Under the Chothia numbering, the CDR sequences of the heavy chain variable domain are set forth in SEQ ID NOs: 43, 44, 45 and CDR sequences of the light chain variable domain are set forth in SEQ ID NOs: 1, 2, 3.

The amino acid sequence for the heavy chain variable region of 1A7 antibody is set forth in SEQ ID NO: 46. The amino acid sequence for the light chain variable region of 1A7 antibody is set forth in SEQ ID NO: 53.

The amino acid sequence for the heavy chain variable region of 1A11 antibody is set forth in SEQ ID NO: 92. The amino acid sequence for the light chain variable region of 1A11 antibody is set forth in SEQ ID NO: 53.

The amino acid sequence for the heavy chain variable region of 1F2 antibody is set forth in SEQ ID NO: 47. The amino acid sequence for the light chain variable region of 1F2 antibody is set forth in SEQ ID NO: 53.

The amino acid sequence for the heavy chain variable region of 2A5 antibody is set forth in SEQ ID NO: 48. The amino acid sequence for the light chain variable region of 2A5 antibody is set forth in SEQ ID NO: 53.

The amino acid sequence for the heavy chain variable region of 2D11 antibody is set forth in SEQ ID NO: 49. The amino acid sequence for the light chain variable region of 2D11 antibody is set forth in SEQ ID NO: 53.

The amino acid sequence for the heavy chain variable region of 2F5 antibody is set forth in SEQ ID NO: 50. The amino acid sequence for the light chain variable region of 2F5 antibody is set forth in SEQ ID NO: 53.

The amino acid sequence for the heavy chain variable region of 3C4 antibody is set forth in SEQ ID NO: 51. The amino acid sequence for the light chain variable region of 3C4 antibody is set forth in SEQ ID NO: 53.

The amino acid sequence for the heavy chain variable region of 3E6 antibody is set forth in SEQ ID NO: 52. The amino acid sequence for the light chain variable region of 3E6 antibody is set forth in SEQ ID NO: 53.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to any one of SEQ ID NO: 46-52, and 92. In some embodiments, the light chain variable region is at least 80%, 85%, 90%, 91%, 92%, 93%, 94%, 95%, 96%, 97%, 98%, or 99% identical to SEQ ID NO: 53. The heavy chain variable region sequence can be paired with the corresponding light chain variable region sequence, and together they bind to PTK7.

Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol.

8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects. In some embodiments, the variable regions are fully human, e.g., derived from human heavy chain immunoglobulin locus sequences (e.g., recombination of human IGHV, human IGHD, and human IGHJ genes), and/or human kappa chain immunoglobulin locus sequences (e.g., recombination of human IGKV and human IGKJ genes).

Furthermore, in some embodiments, the antibodies or antigen-binding fragments thereof described herein can also contain one, two, or three heavy chain variable region CDRs selected from the group of SEQ ID NOs: 4-6, SEQ ID NOs: 7-9, SEQ ID NOs: 10-12, SEQ ID NOs: 13-15, SEQ ID NOs: 16-18, SEQ ID NOs: 19-21, SEQ ID NOs: 22-24, SEQ ID NOs: 25-27, SEQ ID NOs: 28-30, SEQ ID NOs: 31-33, SEQ ID NOs: 34-36, SEQ ID NOs: 37-39, SEQ ID NOs: 40-42, SEQ ID NOs: 43-45, SEQ ID NOs: 86-88, SEQ ID NOs: 89-91; and/or one, two, or three light chain variable region CDRs selected from the group of SEQ ID NOs: 1-3.

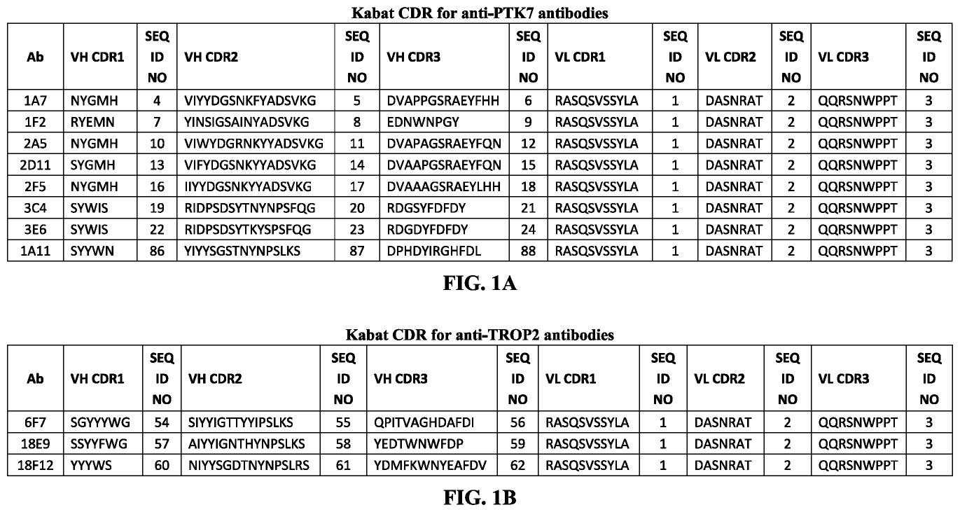

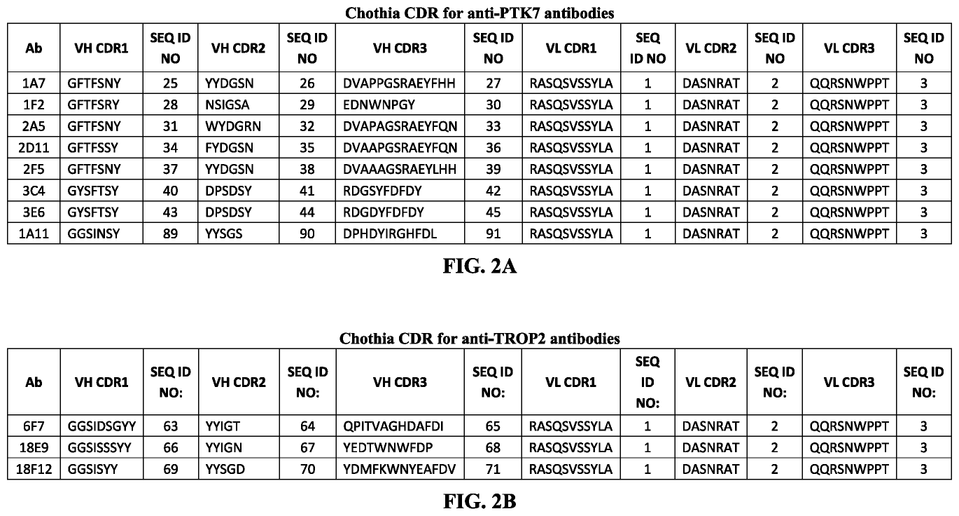

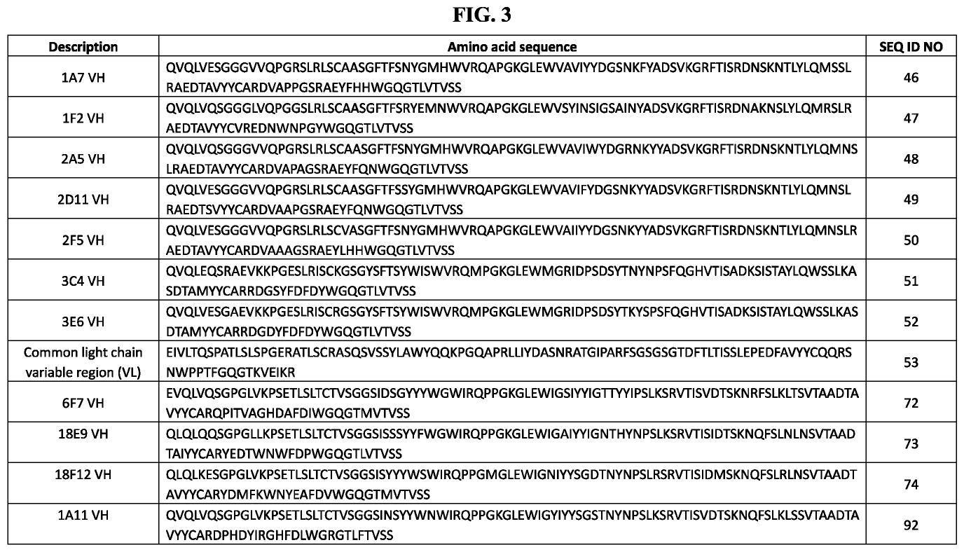

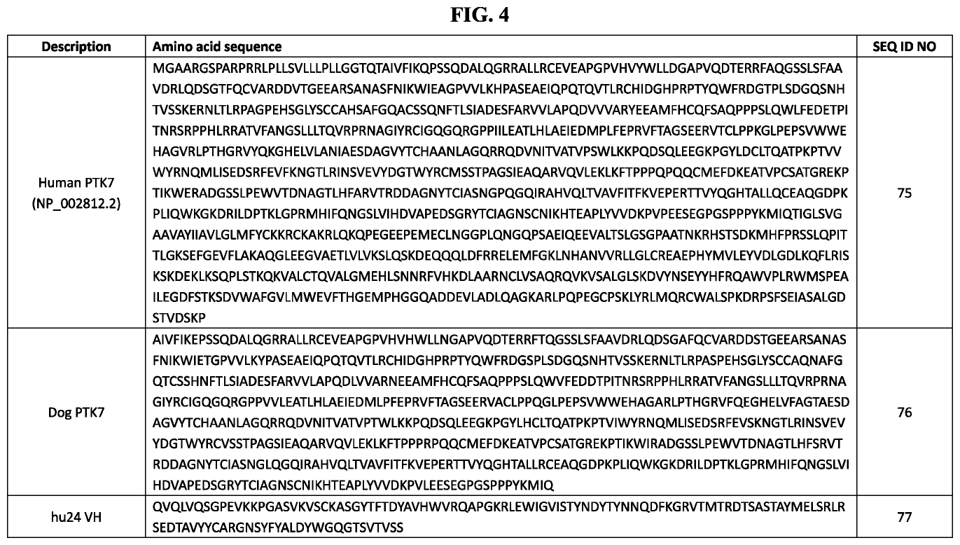

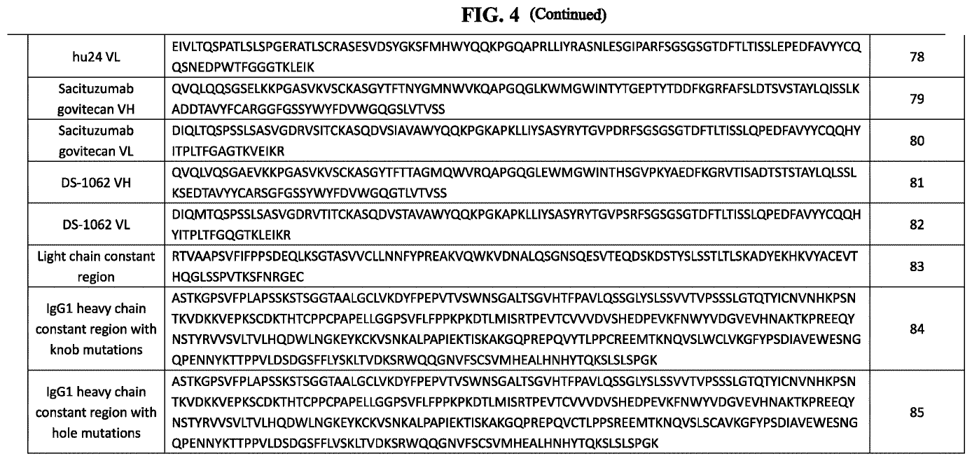

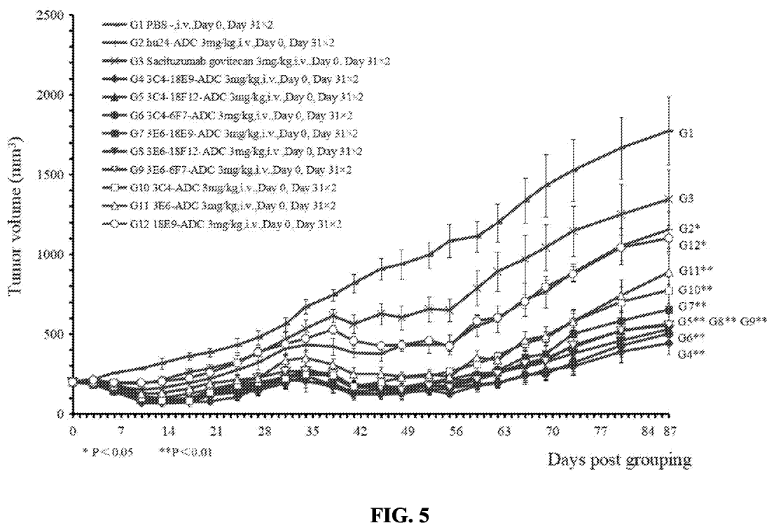

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibody can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 1A (Kabat CDR) and FIG. 2A (Chothia CDR).

In some embodiments, the antibody or an antigen-binding fragment described herein can contain a heavy chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 37 with zero, one or two amino acid insertions, deletions, or substitutions; SEQ ID NO: 38 with zero, one or two amino acid insertions, deletions, or substitutions; SEQ ID NO: 39 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 40 with zero, one or two amino acid insertions, deletions, or substitutions; SEQ ID NO: 41 with zero, one or two amino acid insertions, deletions, or substitutions; SEQ ID NO: 42 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 43 with zero, one or two amino acid insertions, deletions, or substitutions; SEQ ID NO: 44 with zero, one or two amino acid insertions, deletions, or substitutions; SEQ ID NO: 45 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 86 with zero, one or two amino acid insertions, deletions, or substitutions; SEQ ID NO: 87 with zero, one or two amino acid insertions, deletions, or substitutions; SEQ ID NO: 88 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 89 with zero, one or two amino acid insertions, deletions, or substitutions; SEQ ID NO: 90 with zero, one or two amino acid insertions, deletions, or substitutions; SEQ ID NO: 91 with zero, one or two amino acid insertions, deletions, or substitutions.

In some embodiments, the antibody or an antigen-binding fragment described herein can contain a light chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination of Kabat and Chothia numbering scheme.

The disclosure also provides antibodies or antigen-binding fragments thereof that bind to PTK7. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 46, and the selected VL sequence is SEQ ID NO: 53. In some embodiments, the selected VH sequence is SEQ ID NO: 47 and the selected VL sequence is SEQ ID NO: 53. In some embodiments, the selected VH sequence is SEQ ID NO: 48, and the selected VL sequence is SEQ ID NO: 53. In some embodiments, the selected VH sequence is SEQ ID NO: 49, and the selected VL sequence is SEQ ID NO: 53. In some embodiments, the selected VH sequence is SEQ ID NO: 50, and the selected VL sequence is SEQ ID NO: 53. In some embodiments, the selected VH sequence is SEQ ID NO: 51, and the selected VL sequence is SEQ ID NO: 53. In some embodiments, the selected VH sequence is SEQ ID NO: 52, and the selected VL sequence is SEQ ID NO: 53. In some embodiments, the selected VH sequence is SEQ ID NO: 92, and the selected VL sequence is SEQ ID NO: 53.

The disclosure also provides antibodies or antigen-binding fragments thereof that can compete with the antibodies described herein. In some aspects, the antibodies or antigen-binding fragments can bind to the same epitope as the antibodies described herein.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 1A or FIG. 2A, or have sequences as shown in FIG. 3. When the polypeptides are paired with corresponding polypeptide (e.g., a corresponding heavy chain variable region or a corresponding light chain variable region), the paired polypeptides bind to PTK7 (e.g., human PTK7).

The anti-PTK7 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to PTK7 will retain an ability to bind to PTK7. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

In some embodiments, the antibody or antigen-binding fragment thereof described herein recognizes an endogenous PTK7 or a recombinant PTK7. In some embodiments, the antibody or antigen-binding fragment thereof described herein recognizes human PTK7. In some embodiments, the antibody or antigen-binding fragment thereof described herein recognizes monkey PTK7. In some embodiments, the antibody or antigen-binding fragment thereof described herein recognizes dog PTK7.

### Anti-TROP2 Antibodies and Antigen-Binding Fragments

The disclosure provides antibodies and antigen-binding fragments thereof that specifically bind to TROP2 (e.g., human TROP2, monkey TROP2, or dog TROP2). The antibodies and antigen-binding fragments described herein are capable of binding to TROP2. These antibodies can be agonists or antagonists. In some embodiments, these antibodies can increase immune response. In some embodiments, these antibodies can block TROP2 activity, e.g., the stimulation of calcium release and the activation of the MAPK signaling pathway.

The disclosure provides e.g., anti-TROP2 antibodies 18E9, 18F12, 6F7, the chimeric antibodies thereof, and the human or humanized antibodies thereof.

The CDR sequences for 18E9, and 18E9 derived antibodies (e.g., human or humanized antibodies) include CDRs of the heavy chain variable domain, SEQ ID NOs: 57, 58, 59, and CDRs of the light chain variable domain, SEQ ID NOs: 1, 2, 3 as defined by Kabat numbering. The CDRs can also be defined by Chothia system. Under the Chothia numbering, the CDR sequences of the heavy chain variable domain are set forth in SEQ ID NOs: 66, 67, 68 and CDR sequences of the light chain variable domain are set forth in SEQ ID NOs: 1, 2, 3.

Similarly, the CDR sequences for 18F12, and 18F12 derived antibodies include CDRs of the heavy chain variable domain, SEQ ID NOs: 60, 61, 62, and CDRs of the light chain variable domain, SEQ ID NOs: 1, 2, 3, as defined by Kabat numbering. Under Chothia numbering, the CDR sequences of the heavy chain variable domain are set forth in SEQ ID NOs: 69, 70, 71, and CDRs of the light chain variable domain are set forth in SEQ ID NOs: 1, 2, 3.

The CDR sequences for 6F7, and 6F7 derived antibodies include CDRs of the heavy chain variable domain, SEQ ID NOs: 54, 55, 56, and CDRs of the light chain variable domain, SEQ ID NOs: 1, 2, 3, as defined by Kabat numbering. Under Chothia numbering, the CDR sequences of the heavy chain variable domain are set forth in SEQ ID NOs: 63, 64, 65, and CDRs of the light chain variable domain are set forth in SEQ ID NOs: 1, 2, 3.

The amino acid sequence for the heavy chain variable region of 18E9 antibody is set forth in SEQ ID NO: 73. The amino acid sequence for the light chain variable region of 18E9 antibody is set forth in SEQ ID NO: 53.

The amino acid sequence for the heavy chain variable region of 18F12 antibody is set forth in SEQ ID NO: 74. The amino acid sequence for the light chain variable region of 18F12 antibody is set forth in SEQ ID NO: 53.

The amino acid sequence for the heavy chain variable region of 6F7 antibody is set forth in SEQ ID NO: 72. The amino acid sequence for the light chain variable region of 6F7 antibody is set forth in SEQ ID NO: 53.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to any one of SEQ ID NOs: 73 and 74. In some embodiments, the light chain variable region is at least 80%, 85%, 90%, 91%, 92%, 93%, 94%, 95%, 96%, 97%, 98%, or 99% identical to SEQ ID NO: 53. The heavy chain variable region sequence can be paired with the corresponding light chain variable region sequence, and together they bind to TROP2.

Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects. In some embodiments, the variable regions are fully human, e.g., derived from human heavy chain immunoglobulin locus sequences (e.g., recombination of human IGHV, human IGHD, and human IGHJ genes), and/or human kappa chain immunoglobulin locus sequences (e.g., recombination of human IGKV and human IGKJ genes).

Furthermore, in some embodiments, the antibodies or antigen-binding fragments thereof described herein can also contain one, two, or three heavy chain variable region CDRs selected from the group of SEQ ID NOs: 54-56, SEQ ID NOs: 57-59, SEQ ID NOs: 60-62, SEQ ID NOs: 63-65, SEQ ID NOs: 66-68, SEQ ID NOs: 69-71; and/or one, two, or three light chain variable region CDRs selected from the group of SEQ ID NOs: 1-3.

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibody can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 1B (Kabat CDR) and FIG. 2B (Chothia CDR).

In some embodiments, the antibody or an antigen-binding fragment described herein can contain a heavy chain variable domain containing one, two, or three of the CDRs of SEQ ID NO: 54 with zero, one or two amino acid insertions, deletions, or substitutions; SEQ ID NO: 55 with zero, one or two amino acid insertions, deletions, or substitutions; SEQ ID NO: 56 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 63 with zero, one or two amino acid insertions, deletions, or substitutions; SEQ ID NO: 64 with zero, one or two amino acid insertions, deletions, or substitutions; SEQ ID NO: 65 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 57 with zero, one or two amino acid insertions, deletions, or substitutions; SEQ ID NO: 58 with zero, one or two amino acid insertions, deletions, or substitutions; SEQ ID NO: 59 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 60 with zero, one or two amino acid insertions, deletions, or substitutions; SEQ ID NO: 61 with zero, one or two amino acid insertions, deletions, or substitutions; SEQ ID NO: 62 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 66 with zero, one or two amino acid insertions, deletions, or substitutions; SEQ ID NO: 67 with zero, one or two amino acid insertions, deletions, or substitutions; SEQ ID NO: 68 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 69 with zero, one or two amino acid insertions, deletions, or substitutions; SEQ ID NO: 70 with zero, one or two amino acid insertions, deletions, or substitutions; SEQ ID NO: 71 with zero, one or two amino acid insertions, deletions, or substitutions.

In some embodiments, the antibody or an antigen-binding fragment described herein can contain a light chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination of Kabat and Chothia numbering scheme.

The disclosure also provides antibodies or antigen-binding fragments thereof that bind to TROP2. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 72, and the selected VL sequence is SEQ ID NO: 53. In some embodiments, the selected VH sequence is SEQ ID NO: 73, and the selected VL sequence is SEQ ID NO: 53. In some embodiments, the selected VH sequence is SEQ ID NO: 74 and the selected VL sequence is SEQ ID NO: 53.

The disclosure also provides antibodies or antigen-binding fragments thereof that can compete with the antibodies described herein. In some aspects, the antibodies or antigen-binding fragments can bind to the same epitope as the antibodies described herein.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 1B or FIG. 2B, or have sequences as shown in FIG. 3. When the polypeptides are paired with corresponding polypeptide (e.g., a corresponding heavy chain variable region or a corresponding light chain variable region), the paired polypeptides bind to TROP2 (e.g., human TROP2, monkey TROP2, or dog TROP2).

The anti-TROP2 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to TROP2 will retain an ability to bind to TROP2. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

In some embodiments, the antibody or antigen-binding fragment thereof described herein recognizes an endogenous TROP2 or a recombinant TROP2. In some embodiments, the antibody or antigen-binding fragment thereof described herein recognizes human TROP2. In some embodiments, the antibody or antigen-binding fragment thereof described herein recognizes monkey TROP2. In some embodiments, the antibody or antigen-binding fragment thereof described herein recognizes dog TROP2.

### Anti-PTK7/TROP2 Multispecific Antibodies and Antigen-Binding Fragments

The disclosure provides multispecific (e.g., bispecific) antibodies and antigen-binding fragments thereof that specifically bind to PTK7/TROP2 (e.g., human PTK7/TROP2). In one aspect, the disclosure provides an anti-PTK7/TROP2 multispecific (e.g., bispecific) antibody or antigen-binding fragment thereof, comprising: a first antigen-binding domain that specifically binds to PTK7; and a second antigen-binding domain that specifically binds to TROP2.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3,
(9) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3,
(10) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively;
(11) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively;
(12) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively;
(13) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively;
(14) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively;
(15) the selected VH1 CDRs 1, 2, 3 amino acid sequences are set forth in SEQ ID NOs: 86-88, respectively, and the selected VL1 CDRs 1, 2, 3 amino acid sequences are set forth in SEQ ID NOs: 1-3, respectively; and
(16) the selected VH1 CDRs 1, 2, 3 amino acid sequences are set forth in SEQ ID NOs: 89-91, respectively, and the selected VL1 CDRs 1, 2, 3 amino acid sequences are set forth in SEQ ID NOs: 1-3, respectively.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3,
(3) the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3,
(4) the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(5) the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively; and
(6) the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the first heavy chain variable region comprises a sequence that is at least 80%, 85%, 90%, 95%, 99%, or 100% identical to SEQ ID NO: 46, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 52, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 46, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 52, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 46, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 52, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 92, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

In some embodiments, the first heavy chain variable region comprises a sequence that is at least 80%, 85%, 90%, 95%, 99%, or 100% identical to SEQ ID NO: 92, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

In some embodiments, the VH1 comprises an amino acid sequence that is at least 80%, 85%, 90%, 95%, 99%, or 100% identical to a selected VH sequence, and the VL1 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 46, and the selected VL sequence is SEQ ID NO: 53;
(2) the selected VH sequence is SEQ ID NO: 47, and the selected VL sequence is SEQ ID NO: 53;
(3) the selected VH sequence is SEQ ID NO: 48, and the selected VL sequence is SEQ ID NO: 53;
(4) the selected VH sequence is SEQ ID NO: 49, and the selected VL sequence is SEQ ID NO: 53;
(5) the selected VH sequence is SEQ ID NO: 50, and the selected VL sequence is SEQ ID NO: 53;
(6) the selected VH sequence is SEQ ID NO: 51, and the selected VL sequence is SEQ ID NO: 53;
(7) the selected VH sequence is SEQ ID NO: 52, and the selected VL sequence is SEQ ID NO: 53; and
(8) the selected VH sequence is SEQ ID NO: 92, and the selected VL sequence is SEQ ID NO: 53.

In some embodiments, the VH2 comprises an amino acid sequence that is at least 80%, 85%, 90%, 95%, 99%, or 100% identical to a selected VH sequence, and the VL2 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 72, and the selected VL sequence is SEQ ID NO: 53;
(2) the selected VH sequence is SEQ ID NO: 73, and the selected VL sequence is SEQ ID NO: 53; and
(3) the selected VH sequence is SEQ ID NO: 74, and the selected VL sequence is SEQ ID NO: 53.

In some embodiments, the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 46, and the selected VL sequence is SEQ ID NO: 53;
(2) the selected VH sequence is SEQ ID NO: 47, and the selected VL sequence is SEQ ID NO: 53;
(3) the selected VH sequence is SEQ ID NO: 48, and the selected VL sequence is SEQ ID NO: 53;
(4) the selected VH sequence is SEQ ID NO: 49, and the selected VL sequence is SEQ ID NO: 53;
(5) the selected VH sequence is SEQ ID NO: 50, and the selected VL sequence is SEQ ID NO: 53;
(6) the selected VH sequence is SEQ ID NO: 51, and the selected VL sequence is SEQ ID NO: 53;
(7) the selected VH sequence is SEQ ID NO: 52, and the selected VL sequence is SEQ ID NO: 53; and
(8) the selected VH sequence is SEQ ID NO: 92, and the selected VL sequence is SEQ ID NO: 53.

In some embodiments, the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 72, and the selected VL sequence is SEQ ID NO: 53;
(2) the selected VH sequence is SEQ ID NO: 73, and the selected VL sequence is SEQ ID NO: 53; and
(3) the selected VH sequence is SEQ ID NO: 74, and the selected VL sequence is SEQ ID NO: 53.

In some embodiments, the first antigen-binding domain specifically binds to human, mouse, monkey, or dog PTK7; and/or the second antigen-binding domain specifically binds to human, mouse, monkey, or dog TROP2.

In some embodiments, the first antigen-binding domain is a human or humanized antigen-binding domain; and/or the second antigen-binding domain is a human or humanized antigen-binding domain.

In some embodiments, the first antigen-binding domain is a single-chain variable fragment (scFv); and/or the second antigen-binding domain is a scFv.

In some embodiments, the first light chain variable region and the second light chain variable region are identical.

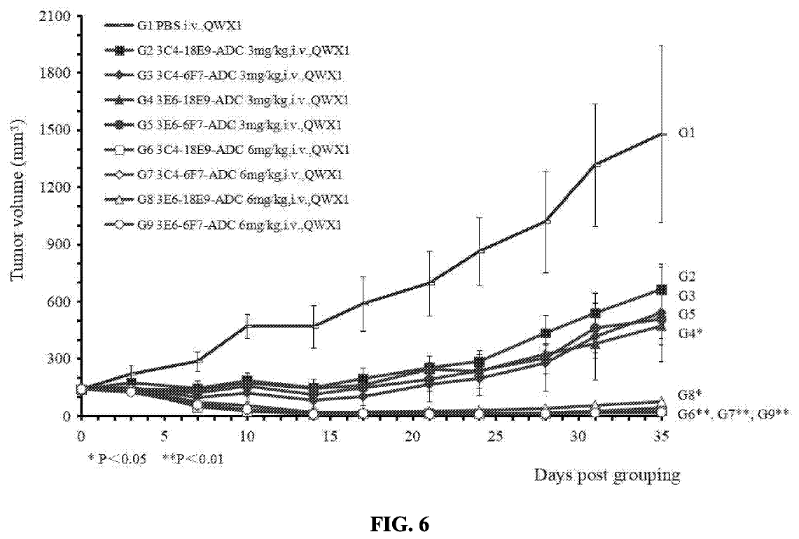

In some embodiments, knobs-into-holes mutations were introduced in the Fc regions of the bispecific antibodies to reduce the chance of wrong pairing between the two heavy chains. Exemplary bispecific antibodies obtained include: 3C4-6F7, 3C4-18E9, 3C4-18F12, 3E6-6F7, 3E6-18E9 and 3E6-18F12 (FIG. 5). The sequence of the human IgG1 constant region with knob mutations is set forth in SEQ ID NO: 84, and the human IgG1 constant region with hole mutations is set forth in SEQ ID NO: 85.

In some embodiments, the anti-PTK7/TROP2 antibodies, or antibody fragments thereof include the combinations of anti-PTK7 and anti-TROP2 antigen-binding domains shown in FIG. 1A-FIG. 3.

In some embodiments, 1A7-6F7 refers to an anti-PTK7/TROP2 antibody that contains a first anti-PTK7 antigen-binding domain that is derived from 1A7 and a second anti-TROP2 antigen-binding domain that is derived from 6F7. In some embodiments, the first anti-PTK7 antigen-binding domain comprises the CDRs of 1A7. In some embodiments, the second anti-TROP2 antigen-binding domain comprises the CDRs of 6F7. In some embodiments, the first anti-PTK7 antigen-binding domain comprises the VH and VL of 1A7. In some embodiments, the second anti-TROP2 antigen-binding domain comprises the VH and VL of 6F7.

In some embodiments, 1A11-6F7 refers to an anti-PTK7/TROP2 antibody that contains a first anti-PTK7 antigen-binding domain that is derived from 1A11 and a second anti-TROP2 antigen-binding domain that is derived from 6F7. In some embodiments, the first anti-PTK7 antigen-binding domain comprises the CDRs of 1A11. In some embodiments, the second anti-TROP2 antigen-binding domain comprises the CDRs of 6F7. In some embodiments, the first anti-PTK7 antigen-binding domain comprises the VH and VL of 1A11. In some embodiments, the second anti-TROP2 antigen-binding domain comprises the VH and VL of 6F7.

In some embodiments, 1F2-6F7 refers to an anti-PTK7/TROP2 antibody that contains a first anti-PTK7 antigen-binding domain that is derived from 1F2 and a second anti-TROP2 antigen-binding domain that is derived from 6F7. In some embodiments, the first anti-PTK7 antigen-binding domain comprises the CDRs of 1F2. In some embodiments, the second anti-TROP2 antigen-binding domain comprises the CDRs of 6F7. In some embodiments, the first anti-PTK7 antigen-binding domain comprises the VH and VL of 1F2. In some embodiments, the second anti-TROP2 antigen-binding domain comprises the VH and VL of 6F7.

In some embodiments, 2A5-6F7 refers to an anti-PTK7/TROP2 antibody that contains a first anti-PTK7 antigen-binding domain that is derived from 2A5 and a second anti-TROP2 antigen-binding domain that is derived from 6F7. In some embodiments, the first anti-PTK7 antigen-binding domain comprises the CDRs of 2A5. In some embodiments, the second anti-TROP2 antigen-binding domain comprises the CDRs of 6F7. In some embodiments, the first anti-PTK7 antigen-binding domain comprises the VH and VL of 2A5. In some embodiments, the second anti-TROP2 antigen-binding domain comprises the VH and VL of 6F7.

In some embodiments, 2D11-6F7 refers to an anti-PTK7/TROP2 antibody that contains a first anti-PTK7 antigen-binding domain that is derived from 2D11 and a second anti-TROP2 antigen-binding domain that is derived from 6F7. In some embodiments, the first anti-PTK7 antigen-binding domain comprises the CDRs of 2D11. In some embodiments, the second anti-TROP2 antigen-binding domain comprises the CDRs of 6F7. In some embodiments, the first anti-PTK7 antigen-binding domain comprises the VH and VL of 2D11. In some embodiments, the second anti-TROP2 antigen-binding domain comprises the VH and VL of 6F7.

In some embodiments, 2F5-6F7 refers to an anti-PTK7/TROP2 antibody that contains a first anti-PTK7 antigen-binding domain that is derived from 2F5 and a second anti-TROP2 antigen-binding domain that is derived from 6F7. In some embodiments, the first anti-PTK7 antigen-binding domain comprises the CDRs of 2F5. In some embodiments, the second anti-TROP2 antigen-binding domain comprises the CDRs of 6F7. In some embodiments, the first anti-PTK7 antigen-binding domain comprises the VH and VL of 2F5. In some embodiments, the second anti-TROP2 antigen-binding domain comprises the VH and VL of 6F7.

In some embodiments, 3C4-6F7 refers to an anti-PTK7/TROP2 antibody that contains a first anti-PTK7 antigen-binding domain that is derived from 3C4 and a second anti-TROP2 antigen-binding domain that is derived from 6F7. In some embodiments, the first anti-PTK7 antigen-binding domain comprises the CDRs of 3C4. In some embodiments, the second anti-TROP2 antigen-binding domain comprises the CDRs of 6F7. In some embodiments, the first anti-PTK7 antigen-binding domain comprises the VH and VL of 3C4. In some embodiments, the second anti-TROP2 antigen-binding domain comprises the VH and VL of 6F7.

In some embodiments, 3E6-6F7 refers to an anti-PTK7/TROP2 antibody that contains a first anti-PTK7 antigen-binding domain that is derived from 3E6 and a second anti-TROP2 antigen-binding domain that is derived from 6F7. In some embodiments, the first anti-PTK7 antigen-binding domain comprises the CDRs of 3E6. In some embodiments, the second anti-TROP2 antigen-binding domain comprises the CDRs of 6F7. In some embodiments, the first anti-PTK7 antigen-binding domain comprises the VH and VL of 3E6. In some embodiments, the second anti-TROP2 antigen-binding domain comprises the VH and VL of 6F7.

In some embodiments, 1A7-18E9 refers to an anti-PTK7/TROP2 antibody that contains a first anti-PTK7 antigen-binding domain that is derived from 1A7 and a second anti-TROP2 antigen-binding domain that is derived from 18E9. In some embodiments, the first anti-PTK7 antigen-binding domain comprises the CDRs of 1A7. In some embodiments, the second anti-TROP2 antigen-binding domain comprises the CDRs of 18E9. In some embodiments, the first anti-PTK7 antigen-binding domain comprises the VH and VL of 1A7. In some embodiments, the second anti-TROP2 antigen-binding domain comprises the VH and VL of 18E9.

In some embodiments, 1A11-18E9 refers to an anti-PTK7/TROP2 antibody that contains a first anti-PTK7 antigen-binding domain that is derived from 1A11 and a second anti-TROP2 antigen-binding domain that is derived from 18E9. In some embodiments, the first anti-PTK7 antigen-binding domain comprises the CDRs of 1A11. In some embodiments, the second anti-TROP2 antigen-binding domain comprises the CDRs of 18E9. In some embodiments, the first anti-PTK7 antigen-binding domain comprises the VH and VL of 1A11. In some embodiments, the second anti-TROP2 antigen-binding domain comprises the VH and VL of 18E9.

In some embodiments, 1F2-18E9 refers to an anti-PTK7/TROP2 antibody that contains a first anti-PTK7 antigen-binding domain that is derived from 1F2 and a second anti-TROP2 antigen-binding domain that is derived from 18E9. In some embodiments, the first anti-PTK7 antigen-binding domain comprises the CDRs of 1F2. In some embodiments, the second anti-TROP2 antigen-binding domain comprises the CDRs of 18E9. In some embodiments, the first anti-PTK7 antigen-binding domain comprises the VH and VL of 1F2. In some embodiments, the second anti-TROP2 antigen-binding domain comprises the VH and VL of 18E9

In some embodiments, 2A5-18E9 refers to an anti-PTK7/TROP2 antibody that contains a first anti-PTK7 antigen-binding domain that is derived from 2A5 and a second anti-TROP2 antigen-binding domain that is derived from 18E9. In some embodiments, the first anti-PTK7 antigen-binding domain comprises the CDRs of 2A5. In some embodiments, the second anti-TROP2 antigen-binding domain comprises the CDRs of 18E9. In some embodiments, the first anti-PTK7 antigen-binding domain comprises the VH and VL of 2A5. In some embodiments, the second anti-TROP2 antigen-binding domain comprises the VH and VL of 18E9.

In some embodiments, 2D11-18E9 refers to an anti-PTK7/TROP2 antibody that contains a first anti-PTK7 antigen-binding domain that is derived from 2D11 and a second anti-TROP2 antigen-binding domain that is derived from 18E9. In some embodiments, the first anti-PTK7 antigen-binding domain comprises the CDRs of 2D11. In some embodiments, the second anti-TROP2 antigen-binding domain comprises the CDRs of 18E9. In some embodiments, the first anti-PTK7 antigen-binding domain comprises the VH and VL of 2D11. In some embodiments, the second anti-TROP2 antigen-binding domain comprises the VH and VL of 18E9.

In some embodiments, 2F5-18E9 refers to an anti-PTK7/TROP2 antibody that contains a first anti-PTK7 antigen-binding domain that is derived from 2F5 and a second anti-TROP2 antigen-binding domain that is derived from 18E9. In some embodiments, the first anti-PTK7 antigen-binding domain comprises the CDRs of 2F5. In some embodiments, the second anti-TROP2 antigen-binding domain comprises the CDRs of 18E9. In some embodiments, the first anti-PTK7 antigen-binding domain comprises the VH and VL of 2F5. In some embodiments, the second anti-TROP2 antigen-binding domain comprises the VH and VL of 18E9.

In some embodiments, 3C4-18E9 refers to an anti-PTK7/TROP2 antibody that contains a first anti-PTK7 antigen-binding domain that is derived from 3C4 and a second anti-TROP2 antigen-binding domain that is derived from 18E9. In some embodiments, the first anti-PTK7 antigen-binding domain comprises the CDRs of 3C4. In some embodiments, the second anti-TROP2 antigen-binding domain comprises the CDRs of 18E9. In some embodiments, the first anti-PTK7 antigen-binding domain comprises the VH and VL of 3C4. In some embodiments, the second anti-TROP2 antigen-binding domain comprises the VH and VL of 18E9.

In some embodiments, 3E6-18E9 refers to an anti-PTK7/TROP2 antibody that contains a first anti-PTK7 antigen-binding domain that is derived from 3E6 and a second anti-TROP2 antigen-binding domain that is derived from 18E9. In some embodiments, the first anti-PTK7 antigen-binding domain comprises the CDRs of 3E6. In some embodiments, the second anti-TROP2 antigen-binding domain comprises the CDRs of 18E9. In some embodiments, the first anti-PTK7 antigen-binding domain comprises the VH and VL of 3E6. In some embodiments, the second anti-TROP2 antigen-binding domain comprises the VH and VL of 18E9.

In some embodiments, 1A7-18F12 refers to an anti-PTK7/TROP2 antibody that contains a first anti-PTK7 antigen-binding domain that is derived from 1A7 and a second anti-TROP2 antigen-binding domain that is derived from 18F12. In some embodiments, the first anti-PTK7 antigen-binding domain comprises the CDRs of 1A7. In some embodiments, the second anti-TROP2 antigen-binding domain comprises the CDRs of 18F12. In some embodiments, the first anti-PTK7 antigen-binding domain comprises the VH and VL of 1A7. In some embodiments, the second anti-TROP2 antigen-binding domain comprises the VH and VL of 18F12.

In some embodiments, 1F2-18F12 refers to an anti-PTK7/TROP2 antibody that contains a first anti-PTK7 antigen-binding domain that is derived from 1F2 and a second anti-TROP2 antigen-binding domain that is derived from 18F12. In some embodiments, the first anti-PTK7 antigen-binding domain comprises the CDRs of 1F2. In some embodiments, the second anti-TROP2 antigen-binding domain comprises the CDRs of 18F12. In some embodiments, the first anti-PTK7 antigen-binding domain comprises the VH and VL of 1F2. In some embodiments, the second anti-TROP2 antigen-binding domain comprises the VH and VL of 18F12.

In some embodiments, 2A5-18F12 refers to an anti-PTK7/TROP2 antibody that contains a first anti-PTK7 antigen-binding domain that is derived from 2A5 and a second anti-TROP2 antigen-binding domain that is derived from 18F12. In some embodiments, the first anti-PTK7 antigen-binding domain comprises the CDRs of 2A5. In some embodiments, the second anti-TROP2 antigen-binding domain comprises the CDRs of 18F12. In some embodiments, the first anti-PTK7 antigen-binding domain comprises the VH and VL of 2A5. In some embodiments, the second anti-TROP2 antigen-binding domain comprises the VH and VL of 18F12.

In some embodiments, 2D11-18F12 refers to an anti-PTK7/TROP2 antibody that contains a first anti-PTK7 antigen-binding domain that is derived from 2D11 and a second anti-TROP2 antigen-binding domain that is derived from 18F12. In some embodiments, the first anti-PTK7 antigen-binding domain comprises the CDRs of 2D11. In some embodiments, the second anti-TROP2 antigen-binding domain comprises the CDRs of 18F12. In some embodiments, the first anti-PTK7 antigen-binding domain comprises the VH and VL of 2D11. In some embodiments, the second anti-TROP2 antigen-binding domain comprises the VH and VL of 18F12.

In some embodiments, 2F5-18F12 refers to an anti-PTK7/TROP2 antibody that contains a first anti-PTK7 antigen-binding domain that is derived from 2F5 and a second anti-TROP2 antigen-binding domain that is derived from 18F12. In some embodiments, the first anti-PTK7 antigen-binding domain comprises the CDRs of 2F5. In some embodiments, the second anti-TROP2 antigen-binding domain comprises the CDRs of 18F12. In some embodiments, the first anti-PTK7 antigen-binding domain comprises the VH and VL of 2F5. In some embodiments, the second anti-TROP2 antigen-binding domain comprises the VH and VL of 18F12.

In some embodiments, 3C4-18F12 refers to an anti-PTK7/TROP2 antibody that contains a first anti-PTK7 antigen-binding domain that is derived from 3C4 and a second anti-TROP2 antigen-binding domain that is derived from 18F12. In some embodiments, the first anti-PTK7 antigen-binding domain comprises the CDRs of 3C4. In some embodiments, the second anti-TROP2 antigen-binding domain comprises the CDRs of 18F12. In some embodiments, the first anti-PTK7 antigen-binding domain comprises the VH and VL of 3C4. In some embodiments, the second anti-TROP2 antigen-binding domain comprises the VH and VL of 18F12.

In some embodiments, 3E6-18F12 refers to an anti-PTK7/TROP2 antibody that contains a first anti-PTK7 antigen-binding domain that is derived from 3E6 and a second anti-TROP2 antigen-binding domain that is derived from 18F12. In some embodiments, the first anti-PTK7 antigen-binding domain comprises the CDRs of 3E6. In some embodiments, the second anti-TROP2 antigen-binding domain comprises the CDRs of 18F12. In some embodiments, the first anti-PTK7 antigen-binding domain comprises the VH and VL of 3E6. In some embodiments, the second anti-TROP2 antigen-binding domain comprises the VH and VL of 18F12.

In some embodiments, in 3C4-6F7, the heavy chain constant region of 3C4 includes knob mutations, and the heavy chain constant region of 6F7 includes hole mutations. In some embodiments. The sequence of the light chain constant region is set forth in SEQ ID NO: 83; the human IgG1 constant region with knob mutations is set forth in SEQ ID NO: 84; and the human IgG1 constant region with hole mutations is set forth in SEQ ID NO: 85.

In some embodiments, the anti-PTK7/TROP2 antibody is a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the anti-PTK7/TROP2 antibodies or antigen-binding fragments thereof described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an anti-PTK7/TROP2 antibody or an antigen-binding fragment in vitro (e.g., in tissue culture or when stored as a pharmaceutical composition) or in vivo (e.g., in a human).

The anti-PTK7/TROP2 antibodies or antigen-binding fragments thereof can also have various forms. Many different formats of bispecific antibodies or antigen-binding fragments thereof are known in the art, and are described e.g., in Suurs, et al. “A review of bispecific antibodies and antibody constructs in oncology and clinical challenges,” Pharmacology & therapeutics (2019), which is incorporated herein by reference in the entirety.

In some embodiments, the anti-PTK7/TROP2 antibody is a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, or a tandem-scFv. In some embodiments, the anti-PTK7/TROP2 antibody is a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, an Intrabody, a dock and lock, a 1mmTAC, an IgG-IgG conjugate, a Cov-X-Body, or a scFv1-PEG-scFv2.

In some embodiments, the anti-PTK7/TROP2 antibody can be a TrioMab. In a TrioMab, the two heavy chains are from different species, wherein different sequences restrict the heavy-light chain pairing.

In some embodiments, the anti-PTK7/TROP2 antibody has two different heavy chains and one common light chain. Heterodimerization of heavy chains can be based on the knobs-into-holes or some other heavy chain pairing technique.

In some embodiments, CrossMAb technique can be used produce bispecific anti-PTK7/TROP2 antibodies. CrossMAb technique can be used enforce correct light chain association in bispecific heterodimeric IgG antibodies, this technique allows the generation of various bispecific antibody formats, including bi-(1+1), tri-(2+1) and tetra-(2+2) valent bispecific antibodies, as well as non-Fc tandem antigen-binding fragment (Fab)-based antibodies. These formats can be derived from any existing antibody pair using domain crossover, without the need for the identification of common light chains, post-translational processing/in vitro chemical assembly or the introduction of a set of mutations enforcing correct light chain association. The method is described in Klein et al., “The use of CrossMAb technology for the generation of bi- and multispecific antibodies.” MAbs. Vol. 8. No. 6. Taylor & Francis, 2016, which is incorporated by reference in its entirety. In some embodiments, the CH1 in the heavy chain and the CL domain in the light chain are swapped.

The anti-PTK7/TROP2 antibody can be a Duobody. The Fab-exchange mechanism naturally occurring in IgG4 antibodies is mimicked in a controlled matter in IgG1 antibodies, a mechanism called controlled Fab exchange. This format can ensure specific pairing between the heavy-light chains.

In Dual-variable-domain antibody (DVD-Ig), additional VH and variable light chain (VL) domain are added to each N-terminus for bispecific targeting. This format resembles the IgG-scFv, but the added binding domains are bound individually to their respective N-termini instead of a scFv to each heavy chain N-terminus.

In scFv-IgG, the two scFv are connected to the C-terminus of the heavy chain (CH3). The scFv-IgG format has two different bivalent binding sites and is consequently also called tetravalent. There are no heavy-chain and light-chain pairing problem in the scFv-IgG.

In some embodiments, the anti-PTK7/TROP2 antibody can have a IgG-IgG format. Two intact IgG antibodies are conjugated by chemically linking the C-terminals of the heavy chains.

The anti-PTK7/TROP2 antibody can also have a Fab-scFv-Fc format. In Fab-scFv-Fc format, a light chain, heavy chain and a third chain containing the Fc region and the scFv are assembled. It can ensure efficient manufacturing and purification.

In some embodiments, the anti-PTK7/TROP2 antibody can be a TF. Three Fab fragments are linked by disulfide bridges. Two fragments target the tumor associated antigen (TAA) and one fragment targets a hapten. The TF format does not have an Fc region.

ADAPTIR has two scFvs bound to each side of an Fc region. It abandons the intact IgG as a basis for its construct, but conserves the Fc region to extend the half-life and facilitate purification.

Dual affinity retargeting (DART) has two peptide chains connecting the opposite fragments, thus VLA with VHB and VLB with VHA, and a sulfur bond at their C-termini fusing them together. In DART, the sulfur bond can improve stability over BiTEs.

In DART-Fc, an Fc region is attached to the DART structure. It can be generated by assembling three chains, two via a disulfide bond, as with the DART. One chain contains half of the Fc region which will dimerize with the third chain, only expressing the Fc region. The addition of Fc region enhances half-life leading to longer effective concentrations, avoiding continuous IV.

In tetravalent DART, four peptide chains are assembled. Basically, two DART molecules are created with half an Fc region and will dimerize. This format has bivalent binding to both targets, thus it is a tetravalent molecule.

Tandem diabody (TandAb) comprises two diabodies. Each diabody consists of an VHA and VLB fragment and a VHA and VLB fragment that are covalently associated. The two diabodies are linked with a peptide chain. It can improve stability over the diabody consisting of two scFvs. It has two bivalent binding sites.

The ScFv-scFv-toxin includes toxin and two scFv with a stabilizing linker. It can be used for specific delivery of payload.

In some embodiments, the anti-PTK7/TROP2 antibody is a bispecific antibody. In some embodiments, the bispecific antibody in present disclosure is designed to be 1+1 (monovalent for each target) and has an IgG1 subtype structure. This can reduce the avidity to cells with low expression levels of PTK7 and TROP2, and increase the avidity to cells that co-express PTK7 and TROP2, to achieve enhanced targeting function.

In some embodiments, the anti-PTK7/TROP2 antibodies or antigen-binding fragments thereof have a light chain constant region that is at least 80%, 85%, 86%, 87%, 88%, 89%, 90%, 91%, 92%, 93%, 94%, 95%, 96%, 97%, 98%, 99% or 100% identical to SEQ ID NO: 83, and a heavy chain constant region that is at least 80%, 85%, 86%, 87%, 88%, 89%, 90%, 91%, 92%, 93%, 94%, 95%, 96%, 97%, 98%, 99% or 100% identical to any one of SEQ ID NOs: 84 and 85.

In some embodiments, the anti-PTK7/TROP2 antibodies include KIH mutations. In some embodiments, the anti-PTK7/TROP2 antibody includes a first antigen-binding domain that specifically binds to PTK7, and a second antigen-binding domain that specifically binds to TROP2. In some embodiments, the first antigen-binding domain includes a heavy chain that including one or more knob mutations (a knob heavy chain), and the second antigen-binding domain includes a heavy chain including one or more hole mutations (a hole heavy chain). In some embodiments, the first antigen-binding domain includes a heavy chain that includes one or more hole mutations (a hole heavy chain), and the second antigen-binding domain that includes a heavy chain including one or more knob mutations (a knob heavy chain). In some embodiments, the anti-PTK7/TROP2 antibody includes a knob heavy chain comprising a constant region that is at least 80%, 85%, 86%, 87%, 88%, 89%, 90%, 91%, 92%, 93%, 94%, 95%, 96%, 97%, 98%, 99% or 100% identical to SEQ ID NO: 84. In some embodiments, the anti-PTK7/TROP2 antibody includes a hole heavy chain comprising a constant region that is at least 80%, 85%, 86%, 87%, 88%, 89%, 90%, 91%, 92%, 93%, 94%, 95%, 96%, 97%, 98%, 99% or 100% identical to SEQ ID NO: 85.

### Antibodies and Antigen Binding Fragments

The present disclosure provides anti-PTK7, anti-TROP2, and/or anti-PTK7/TROP2 antibodies and antigen-binding fragments thereof.

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′) 2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFv has one heavy chain variable domain, and one light chain variable domain. In some embodiments, the scFv has two heavy chain variable domains, and two light chain variable domains.

Single-chain Fv (scFv) or antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′) 2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

One-armed antibodies can have a heavy chain and a light chain, and a heavy chain fragment comprising CH2 and CH3 domains of IgG. In some embodiments, a one-armed antibody is an antibody that only has one of the two antigen binding arms in a typical antibody. In some embodiments, a one-armed antibody comprises an antigen binding arm (e.g., VH+CH1 and VL+CL), and a Fc.

Antibodies and antibody fragments of the present disclosure can be modified in the Fc region to provide desired effector functions or serum half-life. In some embodiments, the Fc region can be modified to silence or decrease complement-dependent cytotoxicity (CDC) or antibody-dependent cellular cytotoxicity (ADCC). In some embodiments, the Fc region can be modified to increase complement-dependent cytotoxicity (CDC) or antibody-dependent cellular cytotoxicity (ADCC).

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94:7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

In some embodiments, sequences (e.g., CDRs or VH/VL sequences) of the antibody or antigen-binding fragment thereof described herein can be used to generate a bispecific antibody targeting PTK7 and TROP2.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

### Antibody Drug Conjugates (ADC)

In some embodiments, the antibodies, the antigen-binding fragments thereof, or the multispecific antibodies (e.g., bispecific antibodies) described herein can be conjugated to a therapeutic agent, optionally with a linker, to form an antibody-drug conjugate. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., monomethyl auristatin E, monomethyl auristatin F, camptothecin, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). In some embodiments, the therapeutic agent is MMAE or MMAF.

Definitions of specific functional groups and chemical terms are described in more detail below. For purpose of this invention, the chemical elements are identified in accordance with the Periodic Table of the Elements, CAS version, Handbook of Chemistry and Physics, 75th Edition, inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modem Methods of Organic Synthesis, 3rd Edition, Cambridge University Press, Cambridge, 1987.

All ranges cited herein are inclusive, unless expressly stated to the contrary. When a range of values is listed, it is intended to encompass each value and sub-range within the range. For example, “C1-6” is intended to encompass, C1, C2, C3, C4, C5, C6, C1-6, C1-5, C1-4, C1-3, C1-2, C2-6, C2-5, C2-4, C2-3, C3-6, C3-5, C3-4, C4-6, C4-5, and C5-6.

The compounds or any formula depicting and describing the compounds of the present disclosure may have one or more chiral (asymmetric) centers. The present invention encompasses all stereoisomeric forms of the compounds or any formula depicting and describing the compounds of the present invention. Centers of asymmetry that are present in the compounds or any formula depicting and describing the compounds of the present invention can all independently of one another have (R) or(S) configuration. When bonds to a chiral carbon are depicted as straight lines in the structural formulas, or when a compound name is recited without an (R) or(S) chiral designation for a chiral carbon, it is understood that both the (R) and(S) configurations of each such chiral carbon, and hence each enantiomer or diastereomer and mixtures thereof, are embraced within the formula or by the name.

The disclosure includes all possible enantiomers and diastereomers and mixtures of two or more stereoisomers, for example mixtures of enantiomers and/or diastereomers, in all ratios. Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism the disclosure includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis.

Unless otherwise stated, the structures depicted herein are also meant to include the compounds that differ only in the presence of one or more isotopically enriched atoms, in other words, the compounds wherein one or more atoms are replaced by atoms having the same atomic number, but an atomic mass or mass number different from the atomic mass or mass number which predominates in nature. Such compounds are referred to as a “isotopic variant”. The present disclosure is intended to include all pharmaceutically acceptable isotopic variants of the compounds or any formula depicting and describing the compounds of the present invention. Examples of isotopes suitable for inclusion in the compounds of the present invention include, but not limited to, isotopes of hydrogen, such as 2H (i.e., D) and 3H; carbon, such as 11C, 13C, and 14C; chlorine, such as 36Cl; fluorine, such as 18F; iodine, such as 123I and 125I; nitrogen, such as 13N and 15N; oxygen, such as 15O, 17O, and 18O; phosphorus, such as 32P; and sulfur, such as 35S. Certain isotopic variants of the compounds or any formula depicting and describing the compounds of the present disclosure, for example those incorporating a radioactive isotope, may be useful in drug and/or substrate tissue distribution studies. Particularly, compounds having the depicted structures that differ only in the replacement with heavier isotopes, such as the replacement of hydrogen by deuterium (2H, or D), can afford certain therapeutic advantages, for example, resulting from greater metabolic stability, increased in vivo half-life, or reduced dosage requirements and, hence, may be utilized in some particular circumstances. Isotopic variants of compounds or any formula depicting and describing the compounds of the present disclosure can generally be prepared by techniques known to those skilled in the art or by processes analogous to those described in the accompanying examples and synthesis using an appropriate isotopically-labeled reagent in place of the non-labeled reagent previously employed.

The compounds as provided herein are described with reference to both generic formulas and specific compounds. In addition, the compounds of the present disclosure may exist in a number of different forms or derivatives, all within the scope of the disclosure. These include, for example, pharmaceutically acceptable salts, tautomers, stereoisomers, racemic mixtures, regioisomers, prodrugs, solvated forms, different crystal forms or polymorphs, and active metabolites, etc.

As used herein, the term “pharmaceutically acceptable salt”, unless otherwise stated, includes salts that retain the biological effectiveness of the free acid/base form of the specified compound and that are not biologically or otherwise undesirable. Pharmaceutically acceptable salts may include salts formed with inorganic bases or acids and organic bases or acids. In cases where the compounds of the present disclosure contain one or more acidic or basic groups, the disclosure also comprises their corresponding pharmaceutically acceptable salts. Thus, the compounds of the present invention which contain acidic groups, such as carboxyl groups, can be present in salt form, and can be used according to the invention, for example, as alkali metal salts, alkaline earth metal salts, aluminum salts or as ammonium salts. More non-limiting examples of such salts include lithium salts, sodium salts, potassium salts, calcium salts, magnesium salts, barium salts, or salts with ammonia or organic amines such as ethylamine, ethanolamine, diethanolamine, triethanolamine, piperidine, N-methylglutamine, or amino acids. These salts are readily available, for instance, by reacting the compound having an acidic group with a suitable base, e.g., lithium hydroxide, sodium hydroxide, sodium propoxide, potassium hydroxide, potassium ethoxide, magnesium hydroxide, calcium hydroxide, or barium hydroxide. Other base salts of compounds of the present disclosure include but are not limited to copper (I), copper (II), iron (II), iron (III), manganese (II), and zinc salts. Compounds of the present disclosure which contain one or more basic groups, e.g., groups which can be protonated, can be present in salt form, and can be used according to the disclosure in the form of their addition salts with inorganic or organic acids. Examples of suitable acids include hydrogen chloride, hydrogen bromide, hydrogen iodide, phosphoric acid, sulfuric acid, nitric acid, methanesulfonic acid, p-toluenesulfonic acid, naphthalenedisulfonic acid, sulfoacetic acid, trifluoroacetic acid, oxalic acid, acetic acid, tartaric acid, lactic acid, salicylic acid, benzoic acid, carbonic acid, formic acid, propionic acid, pivalic acid, diethylacetic acid, malonic acid, succinic acid, pimelic acid, fumaric acid, maleic acid, malic acid, embonic acid, mandelic acid, sulfaminic acid, phenylpropionic acid, gluconic acid, ascorbic acid, isonicotinic acid, citric acid, adipic acid, taurocholic acid, glutaric acid, stearic acid, glutamic acid, or aspartic acid, and other acids known to those skilled in the art. The salts which are formed are, inter alia, hydrochlorides, chlorides, hydrobromides, bromides, iodides, sulfates, phosphates, methanesulfonates (mesylates), tosylates, carbonates, bicarbonates, formates, acetates, sulfoacetates, triflates, oxalates, malonates, maleates, succinates, tartrates, malates, embonates, mandelates, fumarates, lactates, citrates, glutarates, stearates, aspartates, and glutamates. The stoichiometry of the salts formed from the compounds of the disclosure may moreover be an integral or non-integral multiple of one.

Compounds of the present disclosure which contain basic nitrogen-containing groups can be quaternized using agents such as C1-4alkyl halides, for example, methyl, ethyl, isopropyl, and tert-butyl chloride, bromide, and iodide; diC1-4alkyl sulfates, for example, dimethyl, diethyl, and diamyl sulfate; C10-18alkyl halides, for example, decyl, dodecyl, lauryl, myristyl, and stearyl chloride, bromide, and iodide; and arylC1-4alkyl halides, for example, benzyl chloride and phenethyl bromide.

If the compounds of the present disclosure simultaneously contain acidic and basic groups in the molecule, the disclosure also includes, in addition to the salt forms mentioned, inner salts or betaines (zwitterions). The respective salts can be obtained by customary methods which are known to those skilled in the art, for example by contacting these with an organic or inorganic acid or base in a solvent or dispersant, or by anion exchange or cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts. For a review on more suitable salts, see Stahl and Wermuth, Handbook of Pharmaceutical Salts: Properties, Selection, and Use (Wiley-VCH, 2002).

The compound or any formula depicting and describing the compounds of the present disclosure and pharmaceutically acceptable salts thereof may exist in unsolvated and solvated forms. As used herein, the term “solvate” refers to a molecular complex comprising the compound of Formula (I), or a pharmaceutically acceptable salt thereof, and one or more pharmaceutically acceptable solvent molecules. For example, the term “hydrate” is employed when the solvent is water.

Pharmaceutically acceptable solvates in accordance with the present disclosure may include those wherein the solvent of crystallization may be isotopically substituted, e.g., D2O, d6-acetone, d6-DMSO.

### Linker (Linking Agent Compound)

In some embodiments, the therapeutic agent is conjugated via a linker (or a linking agent compound). As used herein, the term “linker” or “linking agent compound” refers to a compound that can connect a ligand (e.g., the antibodies, the antigen-binding fragments thereof, or the antigen-binding protein constructs (e.g., bispecific antibodies) described herein) and a therapeutic agent (e.g., any of the therapeutic agents described herein) together to form a ligand-drug conjugate by reacting with a group of the ligand compound and the therapeutic agent compound respectively by, for example, a coupling reaction.

In some embodiments, the linker described herein is a compound having the following formula:

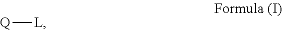

or a pharmaceutically acceptable salt, solvate, stereoisomer, or isotopic variant thereof, wherein Q denotes to a junction moiety capable of being coupled to a ligand via a bond selected from the group consisting of carbonyl, thioether, amide, disulfide and hydrazone bond; L denotes to a linker moiety capable of connecting Q to a therapeutic agent.

In some embodiments, the junction moiety (Q in Formula (I)) has the following structure:

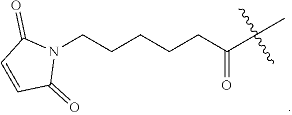 [SMILES: *C(=O)CCCCCN1C(=O)C=CC1=O]

In some embodiments, the linker moiety (L in Formula (I)) has the following formula:

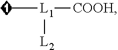

where L1 is a polypeptide residue consisting of three to eight amino acid residues which comprises at least one amino acid residue with a side chain carboxyl group, for example, glutamic acid residue or aspartic acid residue, where “—COOH” denotes carboxyl group of an amino acid residue at C-terminal of the polypeptide residue;
L2 is absent or a monodentate, bidentate or tridentate hydrophilic group attached to the side chain carboxyl group on the amino acid residue of the polypeptide residue L1, and L2 has a structure of —NHC(RL2a)(RL2b)(RL2c), where RL2a, RL2b, and RL2c are each independently selected from the group consisting of H, —(CH2O)(CH2CH2O)m(CH2)pC(O)OH, and —(CH2O) (CH2CH2O)m(CH2)pC(O)NHRL2d, RL2d is H or C1-6 alkyl optionally substituted with 1 to 6 hydroxy groups, each m is independently an integer from 0 to 10, preferably 0 to 4, for example 0, 1, 2, 3, or 4, especially preferably m is 0, and each p is independent an integer from 1 to 4, for example, 1, 2, 3, or 4; and
[symbol] denotes to the N-terminal side of the polypeptide residue covalently attached to the junction moiety Q.

In some embodiments, the polypeptide residue L1 is NH-Glu-Val-Ala-COOH. In some embodiments, the hydrophilic group L2 has the following structure:

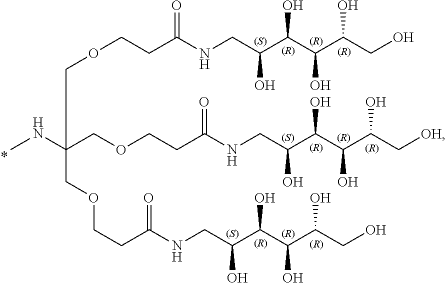

wherein “*” denotes the site covalently attached to polypeptide residue L1, e.g., side chain of the Glu residue in NH-Glu-Val-Ala-COOH.

In some embodiments, the linker described herein is a compound having the following structure:

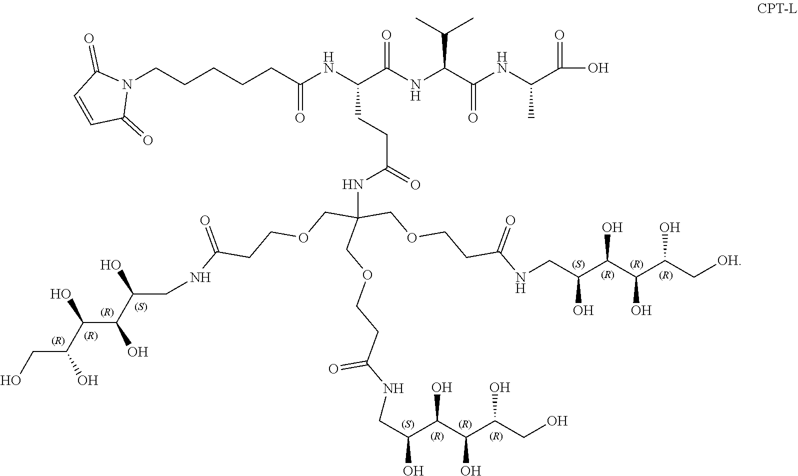

In some embodiments, the linker is a VC linker. Details of the linkers used for ADCs can be found, e.g., in Su, Z. et al. “Antibody-drug conjugates: Recent advances in linker chemistry.” Acta Pharmaceutica Sinica B (2021), which is incorporated herein by reference in its entirety.

### Therapeutic Agent

In some embodiments, the therapeutic agent that is conjugated to the antibodies, the antigen-binding fragments thereof, or the multispecific antibodies (e.g., bispecific antibodies) described herein is discussed as follows.

In some embodiments, the therapeutic agent described herein is a cytotoxic agent. In some embodiments, the cytotoxic agent is a camptothecin compound, an analogue or a derivative thereof. In some preferred embodiments, the camptothecin compound is a compound having the following structure:

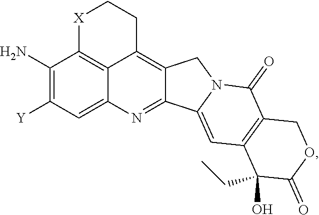

wherein X is selected from the group consisting of —CH2-, O and S; Y is selected from the group consisting of H, D, and F.

In some embodiments, the therapeutic agent is (S)-4-amino-9-ethyl-9-hydroxy-1,9,12,15-tetrahydro-13H-pyrano[3′,4′: 6,7]indolizino[1,2-b]thiopyrano[4,3,2-de]quinoline-10,13(2H)-dione) (CPT-1). The structure of CPT-1 is shown below:

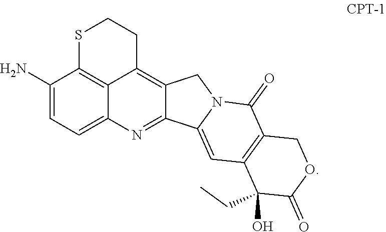 [SMILES: CC[C@@]1(O)C(=O)OCc2c1cc1n(c2=O)Cc2c-1nc1ccc(N)c3c1c2CCS3]

In some embodiments, the therapeutic agent is (S)-4-amino-9-ethyl-9-hydroxy-1,9,12,15-tetrahydro-13H-pyrano[4,3,2-de]pyrano[3′,4′: 6,7]indolizino[1,2-b]quinoline-10,13(2H)-dione (CPT-2). The structure of CPT-2 is shown below:

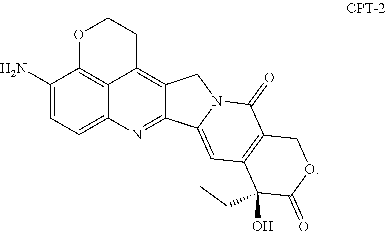 [SMILES: CC[C@@]1(O)C(=O)OCc2c1cc1n(c2=O)Cc2c-1nc1ccc(N)c3c1c2CCO3]

In some embodiments, the therapeutic agent is CPT3. The structure of CPT-3 is shown below:

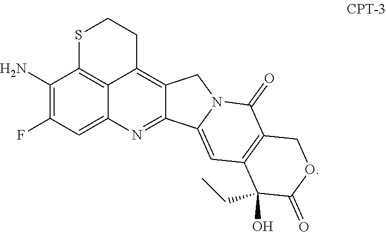 [SMILES: CC[C@@]1(O)C(=O)OCc2c1cc1n(c2=O)Cc2c-1nc1cc(F)c(N)c3c1c2CCS3]

In some embodiments, the therapeutic agent is (S)-4-amino-9-ethyl-5-fluoro-9-hydroxy-1,9,12,15-tetrahydro-13H-pyrano[4,3,2-de]pyrano[3′,4′: 6,7]indolizino[1,2-b]quinoline-10,13(2H)-dione (CPT-4). The structure of CPT-4 is shown below:

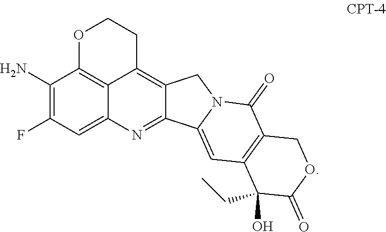 [SMILES: CC[C@@]1(O)C(=O)OCc2c1cc1n(c2=O)Cc2c-1nc1cc(F)c(N)c3c1c2CCO3]

In some embodiments, the therapeutic agent is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the therapeutic agent is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

### Linker-Therapeutic Agent Compound

In some embodiments, a linker (e.g., any of the linkers described herein) and a therapeutic agent (e.g., any of the therapeutic agents described herein) can be linked to form a “linker-therapeutic agent” compound.

In some embodiments, the linker-therapeutic agent compound has the following structure:

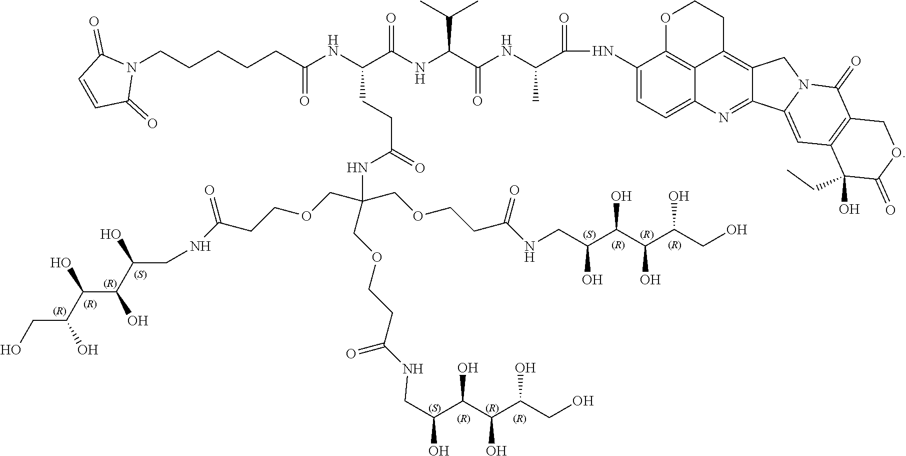

In some embodiments, the linker-therapeutic agent compound has the following structure:

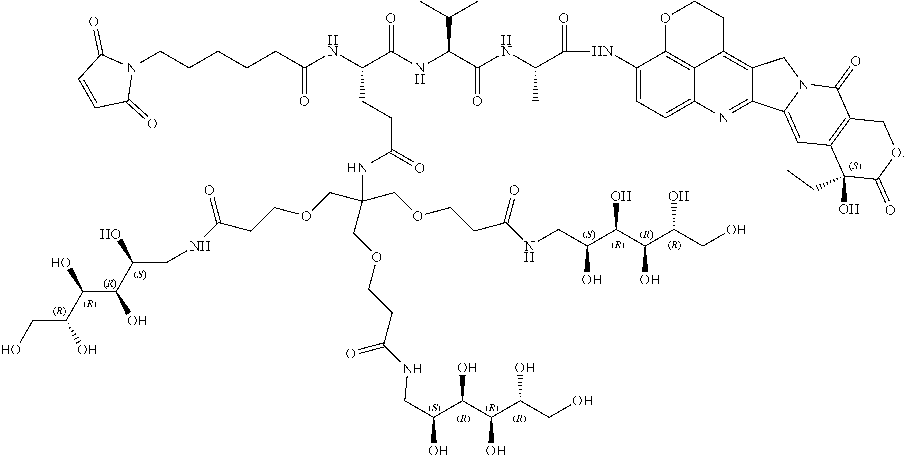

In some embodiments, an antibody (“Ab”), e.g., any of the antibodies, the antigen-binding fragments thereof, or the antigen-binding protein constructs (e.g., bispecific antibodies) described herein, can be linked to a linker-therapeutic agent compound (e.g., any of the linker-therapeutic agent compounds described herein) to generate an antibody-drug conjugate. In some embodiments, the antibody-drug conjugate has the following structure:

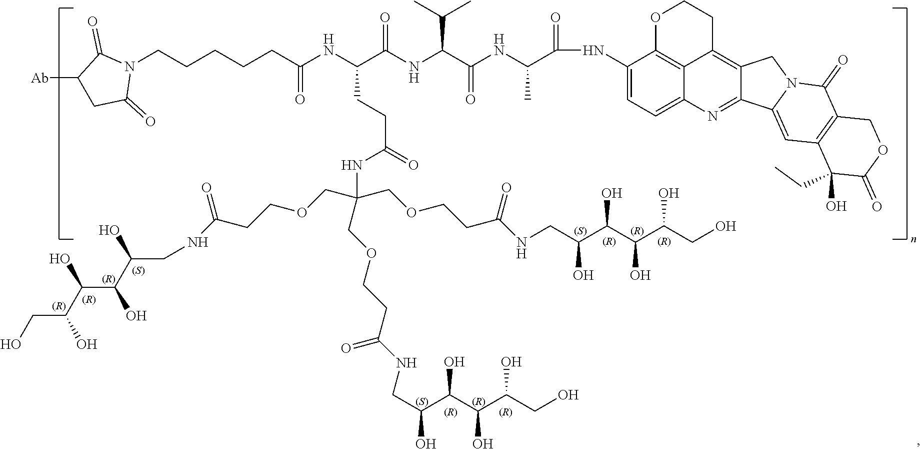

wherein n=1, 2, 3, 4, 5, 6, 7, or 8.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, higher drug loading, e.g. p≥5, may cause aggregation, insolubility, toxicity, or loss of cellular permeability of certain antibody-drug conjugates. In certain embodiments, the average drug loading for an antibody-drug conjugate ranges from 1 to about 8; from about 2 to about 6; or from about 3 to about 5. Indeed, it has been shown that for certain antibody-drug conjugates, the optimal ratio of drug moieties per antibody can be around 4. In some embodiments, the drug-to-antibody ratio (DAR) of the ADCs described herein is about 3.8, about 3.9, about 4.0, about 4.1, about 4.2, about 4.3, about 4.4, about 4.5, about 4.6, about 4.7, about 5.0, about 5.5, about 6.0, about 6.5, about 7.0, about 7.5, about 8.0, about 8.5, or about 9.0. In some embodiments, the DAR of the ADCs described herein is about 3.5 to about 4.5, about 3.6 about 4.5, about 3.7 to about 4.5, about 3.8 to about 4.5, about 3.9 to about 4.5, about 4.0 to about 4.5, about 4.1 to about 4.5, about 4.2 to about 4.5, about 4.3 to about 4.5, about 4.4 to about 4.5, about 3.5 to about 4.4, about 3.6 to about 4.4, about 3.7 to about 4.4, about 3.8 to about 4.4, about 3.9 to about 4.4, about 4.0 to about 4.4, about 4.1 to about 4.4, about 4.2 to about 4.4, about 4.3 to about 4.4, about 3.5 to about 4.3, about 3.6 to about 4.3, about 3.7 to about 4.3, about 3.8 to about 4.3, about 3.9 to about 4.3, about 4.0 to about 4.3, about 4.1 to about 4.3, about 4.2 to about 4.3, about 3.5 to about 4.2, about 3.6 to about 4.2, about 3.7 to about 4.2, about 3.8 to about 4.2, about 3.9 to about 4.2, about 4.0 to about 4.2, about 4.1 to about 4.2, about 3.5 to about 4.1, about 3.6 to about 4.1, about 3.7 to about 4.1, about 3.8 to about 4.1, about 3.9 to about 4.1, about 4.0 to about 4.1, about 3.5 to about 4.0, about 3.6 to about 4.0, about 3.7 to about 4.0, about 3.8 to about 4.0, about 3.9 to about 4.0, about 3.5 to about 3.9, about 3.6 to about 3.9, about 3.7 to about 3.9, about 3.8 to about 3.9, about 3.5 to about 3.8, about 3.6 to about 3.8, about 3.7 to about 3.8, about 3.5 to about 3.7, about 3.6 to about 3.7, or about 3.5 to about 3.6. In some embodiments, the average DAR in the composition is about 1~about 2, about 2~about 3, about 3~about 4, about 3~about 5, about 4~about 5, about 5~about 6, about 6~about 7, or about 7~about 8. In some embodiments, the DAR of the ADCs described herein is about 7.5 to about 8.5, about 7.6 to about 8.5, about 7.7 to about 8.5, about 7.8 to about 8.5, about 7.9 to about 8.5, about 8.0 to about 8.5, about 8.1 to about 8.5, about 8.2 to about 8.5, about 8.3 to about 8.5, about 8.4 to about 8.5, about 7.5 to about 8.4, about 7.6 to about 8.4, about 7.7 to about 8.4, about 7.8 to about 8.4, about 7.9 to about 8.4, about 8.0 to about 8.4, about 8.1 to about 8.4, about 8.2 to about 8.4, about 8.3 to about 8.4, about 7.5 to about 8.3, about 7.6 to about 8.3, about 7.7 to about 8.3, about 7.8 to about 8.3, about 7.9 to about 8.3, about 8.0 to about 8.3, about 8.1 to about 8.3, about 8.2 to about 8.3, about 7.5 to about 8.2, about 7.6 to about 8.2, about 7.7 to about 8.2, about 7.8 to about 8.2, about 7.9 to about 8.2, about 8.0 to about 8.2, about 8.1 to about 8.2, about 7.5 to about 8.1, about 7.6 to about 8.1, about 7.7 to about 8.1, about 7.8 to about 8.1, about 7.9 to about 8.1, about 8.0 to about 8.1, about 7.5 to about 8.0, about 7.6 to about 8.0, about 7.7 to about 8.0, about 7.8 to about 8.0, about 7.9 to about 8.0, about 7.5 to about 7.9, about 7.6 to about 7.9, about 7.7 to about 7.9, about 7.8 to about 7.9, about 7.5 to about 7.8, about 7.6 to about 7.8, about 7.7 to about 7.8, about 7.5 to about 7.7, about 7.6 to about 7.7, or about 7.5 to about 7.6.

In some embodiments, the anti-PTK7, anti-TROP2, and/or anti-PTK7/TROP2 ADC described herein can effectively inhibit in vitro cancer cell growth at a concentration of less than 10 μg/mL, less than 3.33 μg/mL, less than 1.11 μg/mL, less than 0.37 μg/mL, less than 0.12 μg/mL, less than 0.04 μg/mL, or less than 0.01 μg/mL. In some embodiments, the anti-PTK7, anti-TROP2, and/or anti-PTK7/TROP2 ADC described herein can inhibit in vivo cancer cell growth (e.g., lung cancer, gastric cancer, or skin cancer) in a xenograft mouse model at a dose level of less than 10 mg/kg, 9 mg/kg, 8 mg/kg, 7 mg/kg, 6 mg/kg, 5 mg/kg, 4 mg/kg, 3 mg/kg, 2 mg/kg, 1.5 mg/kg, or 1 mg/kg.

### Antibody and ADC Characteristics

In some embodiments, the antibodies or antigen-binding fragments thereof described herein or ADC derived therefrom can block the binding between PTK7 and PTK7 ligands (e.g., Wnt ligand). In some embodiments, the antibodies or antigen-binding fragments thereof described herein or ADC derived therefrom can block the binding between TROP2 and TROP2 ligands (e.g., ErbB3-ligand neuregulin-1). In some embodiments, the antibodies or antigen-binding fragments thereof described herein or ADC derived therefrom can block the binding between PTK7 and PTK7 ligands and the binding between TROP2 and TROP2 ligands.

The antibodies or antigen-binding fragments thereof as described herein or ADC derived therefrom can be an agonist or antagonist. In some embodiments, by binding to PTK7, the antibody can inhibit PTK7 signaling pathway. In some embodiments, by binding to TROP2, the antibody can inhibit TROP2 signaling pathway. In some embodiments, by binding to PTK7 and TROP2, the antibody can inhibit PTK7 signaling pathway and TROP2 pathway. In some embodiments, the antibody can upregulate immune response or downregulate immune response.

In some embodiments, the antibody (or antigen-binding fragments thereof) or ADC derived therefrom specifically binds to PTK7 (e.g., human PTK7, monkey PTK7 (e.g., rhesus macaques, Macaca fascicularis), dog PTK7, mouse PTK7) with a dissociation rate (koff) of less than 0.1 s⁻¹, less than 0.01 s⁻¹, less than 0.001 s⁻¹, less than 0.0001 s⁻¹, less than 0.00001 s⁻¹, less than 0.000001 s⁻¹ or less than 0.0000001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, greater than 0.000001 s⁻¹, greater than 0.0000001 s⁻¹ or greater than 0.00000001 s⁻¹.

In some embodiments, the antibody (or antigen-binding fragments thereof) or ADC derived therefrom specifically binds to TROP2 (e.g., human TROP2, monkey TROP2 (e.g., rhesus macaques, Macaca fascicularis), dog TROP2, mouse TROP2) with a dissociation rate (koff) of less than 0.1 s⁻¹, less than 0.01 s⁻¹, less than 0.001 s⁻¹, less than 0.0001 s⁻¹, less than 0.00001 s⁻¹, less than 0.000001 s⁻¹ or less than 0.0000001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, greater than 0.000001 s⁻¹, greater than 0.0000001 s⁻¹ or greater than 0.00000001 s⁻¹.

In some embodiments, kinetic association rates (kon) for PTK7 or TROP2 is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD for PTK7 or TROP2 is less than 1×10⁻⁶ M, less than 1×10⁻⁷M, less than 1×10⁻⁸ M, less than 1×10⁻⁹ M, less than 1×10⁻¹⁰ M, less than 1×10⁻¹¹ M, less than 1×10⁻¹² M, less than 1×10⁻¹³ M or less than 1×10⁻¹⁴ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, greater than 1×10⁻¹² M, greater than 1×10⁻¹³ M, greater than 1×10⁻¹⁴ M.

General techniques for measuring the affinity of an antibody for an antigen include, e.g., ELISA, RIA, and surface plasmon resonance (SPR). In some embodiments, the antibody binds to human PTK7 (SEQ ID NO: 75), monkey PTK7, dog PTK7 (SEQ ID NO: 76), and/or mouse PTK7. In some embodiments, the antibody does not bind to human PTK7, monkey PTK7, dog PTK7, and/or mouse PTK7.

In some embodiments, the antibody binds to human TROP2, monkey TROP2, dog TROP2, and/or mouse TROP2. In some embodiments, the antibody does not bind to human TROP2, monkey TROP2, dog TROP2, and/or mouse TROP2.

In some embodiments, the antibody binds to human PTK7 and TROP2 (PTK7/TROP2), monkey PTK7/TROP2, dog PTK7/TROP2, and/or mouse PTK7/TROP2. In some embodiments, the antibody does not bind to human PTK7/TROP2, monkey PTK7/TROP2, dog PTK7/TROP2, and/or mouse PTK7/TROP2.

In some embodiments, the antibody or antigen-binding fragment thereof as described herein or ADC derived therefrom is added to NUGC-4 cells (Cobioer, Cat #: CBP60493) or LN229 cells (ATCC, Cat #: CRL-2611) to test the endocytosis rate. In some embodiments, the antibody or antigen-binding fragment thereof as described herein or ADC derived therefrom has an endocytosis rate of above 5%, above 10%, above 15%, above 20%, above 25%, above 30%, above 35%, above 40%, above 45%, above 50%, above 55%, above 60%, above 65%, above 70%, above 75%, above 80%, above 85%, above 90%, above 91%, above 92%, above 93%, above 94%, above 95%, above 96%, above 97%, or above 98%.

In some embodiments, thermal stabilities are determined. The antibodies or antigen binding fragments as described herein or ADC derived therefrom can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies or antigen-binding fragments thereof as described herein or ADC derived therefrom can bind to the same epitope of PTK7, TROP2 and/or PTK7/TROP2. In some embodiments, the antibodies or antigen-binding fragments thereof as described herein or ADC derived therefrom can bind to different epitopes of PTK7, TROP2 and/or PTK7/TROP2.

In some embodiments, the ADC described herein has an average drug-to-antibody ratio (DAR) of higher than 3, higher than 3.2, higher than 3.4, higher than 3.6, higher than 3.8, higher than 4, higher than 4.2, higher than 4.4, or higher than 4.6, as determined by HPLC. In some embodiments, the ADC described herein has an average DAR of lower than 3, lower than 3.2, lower than 3.4, lower than 3.6, lower than 3.8, lower than 4, lower than 4.2, lower than 4.4 or lower than 4.6, as determined by HPLC.

In some embodiments, the antibody or antigen-binding fragment thereof as described herein or ADC derived therefrom has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody or antigen-binding fragment thereof as described herein or ADC derived therefrom has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$TGI ( \% ) = [ 1 - ( Ti - T 0 ) / ( Vi - V 0 ) ] \times 100 \%$$

Ti is the average tumor volume in the treatment group on day i. TO is the average tumor volume in the treatment group on day zero. Vi is the average tumor volume in the control group on day i. V0 is the average tumor volume in the control group on day zero.

In some embodiments, the antibodies or antigen-binding fragments thereof as described herein or ADC derived therefrom are PTK7 antagonist. In some embodiments, the antibodies or antigen binding fragments as described herein or ADC derived therefrom decrease PTK7 signal transduction in a target cell that expresses PTK7.

In some embodiments, the antibodies or antigen-binding fragments thereof as described herein or ADC derived therefrom are TROP2 antagonist. In some embodiments, the antibodies or antigen binding fragments as described herein or ADC derived therefrom decrease TROP2 signal transduction in a target cell that expresses TROP2.

In some embodiments, the antibodies or antigen-binding fragments thereof as described herein or ADC derived therefrom are PTK7 and TROP2 (PTK7/TROP2) antagonist. In some embodiments, the antibodies or antigen binding fragments as described herein or ADC derived therefrom decrease PTK7 and TROP2 (PTK7/TROP2) signal transduction in a target cell that expresses PTK7 and TROP2 (PTK7/TROP2).

In some embodiments, the antibodies or antigen binding fragments as described herein or ADC derived therefrom can enhance APC (e.g., DC cell) function, for example, inducing surface expression of costimulatory and MHC molecules, inducing production of proinflammatory cytokines, and/or enhancing T cell triggering function.

In some embodiments, the antibodies or antigen binding fragments as described herein or ADC derived therefrom can bind to tumor cells that express PTK7. In some embodiments, the antibodies or antigen binding fragments as described herein or ADC derived therefrom can bind to tumor cells that express TROP2. In some embodiments, the antibodies or antigen binding fragments as described herein or ADC derived therefrom can bind to tumor cells that express PTK7 and TROP2. In some embodiments, the antibodies or antigen binding fragments as described herein or ADC derived therefrom can induce complement-dependent cytotoxicity (CDC) and/or antibody dependent cellular cytoxicity (ADCC), and kill the tumor cell.

In some embodiments, the antibodies or antigen binding fragments as described herein or ADC derived therefrom have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis.

In some embodiments, the antibodies or antigen binding fragments as described herein or ADC derived therefrom can induce complement complement-dependent cytotoxicity (CDC).

In some embodiments, the Fc region is human IgG1, human IgG2, human IgG3, or human IgG4. In some embodiments, the antibody is a human IgG1 antibody, optionally with SI mutations, LALA mutations, N297A mutation, YTE mutations, and/or FLAA mutations. In some embodiments, the antibody is a human IgG4 antibody, optionally with SI mutations, LALA mutations, N297A mutation, YTE mutations, and/or FLAA mutations.

In some embodiments, the antibodies or antigen binding fragments as described herein or ADC derived therefrom do not have a functional Fc region. For example, the antibodies or antigen binding fragments are Fab, Fab′, F(ab′) 2, and Fv fragments. In some embodiments, the Fc region has LALA mutations (L234A and L235A mutations according to EU numbering), or LALA-PG mutations (L234A, L235A, P329G mutations according to EU numbering). In some embodiments, the Fc region has FLAA mutations (F234A and L235A according to EU numbering). In some embodiments, the Fc has SI mutations (S239D and 1332E mutations according to EU numbering). In some embodiments, the Fc has N297A mutation according to EU numbering. In some embodiments, the Fc has YTE mutations (M252Y, S254T and T256E according to EU numbering).

Methods of Making Anti-PTK7, Anti-TROP2, and/or Anti-PTK7/TROP2 Antibodies

An isolated fragment of human PTK7 and/or TROP2 can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of PTK7, TROP2 and encompasses an epitope of the protein such that an antibody raised against the peptide forms a specific immune complex with the protein. As described above, the full length sequences of human PTK7 (SEQ ID NO: 75) and TROP2 are known in the art. In some embodiments, an Fc-tagged or His-tagged human PTK7 or TROP2 protein is used as the immunogen.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human PTK7 or TROP2). The preparation can further include an adjuvant, such as Freund's complete or incomplete adjuvant, or a similar immunostimulatory agent.

Polyclonal antibodies can be prepared as described above by immunizing a suitable subject with a PTK7 or TROP2 polypeptide, or an antigenic peptide thereof (e.g., part of PTK7 or TROP2) as an immunogen. The antibody titer in the immunized subject can be monitored over time by standard techniques, such as with an enzyme-linked immunosorbent assay (ELISA) using the immobilized PTK7 or TROP2 polypeptide or peptide. If desired, the antibody molecules can be isolated from the mammal (e.g., from the blood) and further purified by well-known techniques, such as protein A or protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., PTK7 or TROP2. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al., J. Immunol., 151:2296 (1993); Chothia et al., J. Mol. Biol., 196:901 (1987)).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-PTK7, anti-TROP2 or anti-PTK7/TROP2 antibody will contain an amino acid sequence having at least 75%, 80%, 85%, 90%, 95%, 96%, 97%, 98%, or 99% percent identity with a sequence present in the light or heavy chain of the original antibody.

In some embodiments, a mouse (e.g., RenMab™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode the light chains of antibodies (kappa chain). The kappa chain immunoglobulin locus can include e.g., human IGKV (variable) genes, human IGKJ (joining) genes, and mouse light chain constant domain genes. A detailed description regarding RenMab™ mice can be found in PCT/CN2020/075698 or US20200390073A1, which is incorporated herein by reference in its entirety.

In some embodiments, a mouse (e.g., RenLite™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode a common light chain. The kappa chain immunoglobulin locus can include e.g., a human IGKV (variable) gene, a human IGKJ (joining) gene, and mouse light chain constant domain genes. A detailed description regarding RenLite™ mice can be found in PCT/CN2021/097652, which is incorporated herein by reference in its entirety.

The antibodies generated by the mice have a full human VH, a full human VL, and mouse constant regions. In some embodiments, the human VH and human VL is linked to a human IgG constant region (e.g., IgG1, IgG2, IgG3, and IgG4).

Identity or homology with respect to an original sequence is usually the percentage of amino acid residues present within the candidate sequence that are identical with a sequence present within the human, humanized, or chimeric anti-PTK7, anti-TROP2, or anti-PTK7/TROP2 antibody or fragment, after aligning the sequences and introducing gaps, if necessary, to achieve the maximum percent sequence identity, and not considering any conservative substitutions as part of the sequence identity.

Additional modifications to the anti-PTK7, anti-TROP2, or anti-PTK7/TROP2 antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the anti-PTK7, anti-TROP2, or anti-PTK7/TROP2 antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods in Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antigen-binding fragments thereof or ADC derived therefrom of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof or ADC derived therefrom disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, esophageal cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the cancer is NSCLC, ovarian cancer, melanoma, colorectal cancer, breast cancer, a hematological malignancy, head and neck cancer, gastrointestinal cancer, bladder cancer, or bone cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors. In some embodiments, the cancer is colorectal cancer, gastric cancer, breast cancer, lung cancer, melanoma, ovarian cancer, head and neck cancer, pancreatic cancer, endometrial carcinoma, thyroid carcinoma or cervical cancer.

In some embodiments, the compositions and methods disclosed herein can be used for treatment of patients at risk for a cancer. Patients with cancer can be identified with various methods known in the art.

In one aspect, the disclosure provides methods for treating, preventing, or reducing the risk of developing disorders associated with an abnormal or unwanted immune response, e.g., an autoimmune disorder. These autoimmune disorders include, but are not limited to, Alopecia areata, lupus, ankylosing spondylitis, Meniere's disease, antiphospholipid syndrome, mixed connective tissue disease, autoimmune Addison's disease, multiple sclerosis, autoimmune hemolytic anemia, myasthenia gravis, autoimmune hepatitis, pemphigus vulgaris, Behcet's disease, pernicious anemia, bullous pemphigoid, polyarthritis nodosa, cardiomyopathy, polychondritis, celiac sprue-dermatitis, polyglandular syndromes, chronic fatigue syndrome (CFIDS), polymyalgia rheumatica, chronic inflammatory demyelinating, polymyositis and dermatomyositis, chronic inflammatory polyneuropathy, primary agammaglobulinemia, Churg-Strauss syndrome, primary biliary cirrhosis, cicatricial pemphigoid, psoriasis, CREST syndrome, Raynaud's phenomenon, cold agglutinin disease, Reiter's syndrome, Crohn's disease, Rheumatic fever, discoid lupus, rheumatoid arthritis, Cryoglobulinemia sarcoidosis, fibromyalgia, scleroderma, Grave's disease, Sjögren's syndrome, Guillain-Barre, stiff-man syndrome, Hashimoto's thyroiditis, Takayasu arteritis, idiopathic pulmonary fibrosis, temporal arteritis/giant cell arteritis, idiopathic thrombocytopenia purpura (ITP), ulcerative colitis, IgA nephropathy, uveitis, diabetes (e.g., Type I), vasculitis, lichen planus, and vitiligo. The anti-PTK7, anti-TROP2, or anti-PTK7/TROP2 antibodies or antigen-binding fragments thereof or ADC derived therefrom can also be administered to a subject to treat, prevent, or reduce the risk of developing disorders associated with an abnormal or unwanted immune response associated with cell, tissue or organ transplantation, e.g., renal, hepatic, and cardiac transplantation, e.g., graft versus host disease (GVHD), or to prevent allograft rejection. In some embodiments, the subject has dermatological disorders, liver disease (e.g., cirrhosis), Hidradenitis, experimental autoimmune encephalomyelitis. In some embodiments, the subject has renal disease, lupus, Sjogren's syndrome, ulcerative colitics, psoriasis, Hidradenitis suppurativa, Immune Thrombocytopenia (ITP), or other inflammatory arthritis. In some embodiments, the subject has multiple sclerosis or myasthenia gravis. In some embodiments, the subject has Crohn's disease, ulcerative colitis or type 1 diabetes. In some embodiments, the subject has autoimmune thyroid disease, Grave's disease, multiple sclerosis, psoriasis, inflammatory bowel disease (e.g., Crohn's Disease (CD) and ulcerative colitis), rheumatoid arthritis, Sjögren's syndrome, autoimmune nephritis, or systemic lupus erythematosus. In some embodiments, the methods involve administering to the subject an effective amount of a composition as described herein.

As used herein, by an “effective amount” is meant an amount or dosage sufficient to effect beneficial or desired results including halting, slowing, retarding, or inhibiting progression of a disease, e.g., an autoimmune disease or a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment or ADC derived therefrom is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment or ADC derived therefrom may vary, depending on, inter alia, patient history as well as other factors such as the type (and/or dosage) of antibody used.

Effective amounts and schedules for administering the antibodies, ADC, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, ADC, antibody-encoding polynucleotides, and/or compositions disclosed herein, the route of administration, the particular type of antibodies, ADC, antibody-encoding polynucleotides, antigen binding fragments, and/or compositions disclosed herein used and other drugs being administered to the mammal.

A typical daily dosage of an effective amount of an antibody or an ADC is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1 (IDO1) (e.g., epacadostat).

In some embodiments, the additional therapeutic agent can comprise one or more inhibitors selected from the group consisting of an inhibitor of PTK7, an inhibitor or TROP2, an inhibitor of PTK7 and TROP2, an inhibitor of heregulin, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, sorafenib, Votrient, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, a HER2 agonist and a Heregulin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, anti-ICOS antibody, anti-CD27 antibody, anti-OX40 antibody, anti-4-1BB antibody, anti-CD40 antibody, anti-VEGFR2 antibody, anti-EGFR antibody, anti-HER2 antibody, and/or an anti-GITR antibody.

In one aspect, the disclosure provides a combination therapy. In some embodiments, the anti-PTK7, anti-TROP2, or anti-PTK7/TROP2 antibody or antigen-binding fragment thereof (e.g., any antibody described herein) can be administered together with an anti-PD-1 antibody.

### Pharmaceutical Compositions and Routes of Administration

Also provided herein are pharmaceutical compositions that contain at least one (e.g., one, two, three, or four) of the antibodies or antigen-binding fragments described herein or ADC derived therefrom. Two or more (e.g., two, three, or four) of any of the antibodies or antigen-binding fragments described herein or ADC derived therefrom can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein or ADC derived therefrom can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively, antibodies can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein or ADC derived therefrom per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof or ADC derived therefrom for various uses as described herein.

### EXAMPLES

The invention is further described in the following examples, which do not limit the scope of the invention described in the claims.

### Example 1. Anti-PTK7 Antibodies

### Generating Anti-PTK7 Antibodies

To generate antibodies against human PTK7, RenMice (i.e., an engineered mouse comprising DNA encoding human immunoglobulin heavy and kappa light chain variable regions, for example, RenMab™ mice, RenLite™ mice) were immunized with His-tagged human PTK7 protein (Sino Biological, Inc., Cat #: 19399-H08H, containing positions 1-704 of SEQ ID NO: 75) or plasmids encoding PTK7 protein. The antibody immune response was monitored by an antigen-specific immunoassay.

A total of three immunizations were performed. The immunizations were separated by two weeks. One week after the last immunization, retro-orbital blood was collected, and the antibody titer in serum was determined by Fluorescence-Activated Cell Sorting (FACS). Mice with high titer were selected two weeks later for impulse immunizations. Human PTK7 protein or CHO-S cells expressing human PTK7 protein were used for impulse immunizations by intraperitoneal injection and tail vein injection, respectively.

When a desired immune response was achieved, antigen-specific immune cells were isolated from the immunized mice to further obtain anti-PTK7 antibodies or to obtain the light chain and heavy chain variable region sequences of the anti-PTK7 antibodies. For example, single cell technology (for example, using Beacon® Optofluidic System, Berkeley Lights Inc.) was used to screen and find plasma cells that secrete antigen-specific monoclonal antibodies, and reverse transcription and PCR sequencing were used to obtain antibody variable region sequences. The obtained variable region sequences were cloned into a vector containing a sequence encoding the human IgG1 constant region for antibody expression. The binding affinity of the expressed antibody to PTK7 was verified using FACS. Exemplary antibodies obtained included: 1A7, 1A11, 1F2, 2A5, 2D11, 2F5, 3C4 and 3E6.

These antibodies contained substantially the same light chain, and their VH and VL CDR 1-3 sequences are shown in FIG. 1A and FIG. 2A. The VH and the VL regions of 1A7, 1A11, 1F2, 2A5, 2D11, 2F5, 3C4 and 3E6 are shown in FIG. 3.

### Binding Affinity of Anti-PTK7 Antibodies

The affinity of the anti-PTK7 antibodies to His-tagged human PTK7 protein (hPTK7-His, Sino Biological, Inc., Cat #: 19399-H08H), His-tagged cynomolgus monkey PTK7 protein (cPTK7-His, ACRO Biosystems Inc., Cat #: PT7-C52H3) and His-tagged dog PTK7 protein (dPTK7-His, SEQ ID NO: 76) were measured by surface plasmon resonance (SPR) using Biacore™ (Biacore, INC, Piscataway N.J.) 8K biosensor equipped with pre-immobilized Protein A sensor chips.

Purified anti-PTK7 antibodies were diluted to 1 μg/mL and then injected into the Biacore™ 8K biosensor at 10 μL/min for about 50 seconds to achieve a desired protein density (e.g., about 50 response units (RU)). The His-tagged PTK7 protein at a concentration of 200 nM or 0 nM was then injected at 30 μL/min for 180 seconds. Dissociation was monitored for 400 seconds. The chip was regenerated after the last injection of each titration with glycine (pH 2.0, 30 μL/min for 30 seconds).

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below.

TABLE 1

| Antibody | Protein | kon (1/Ms) | koff (1/s) | KD (M) |
|---|---|---|---|---|
| hu24 analog | hPTK7-His | 9.78E+04 | 6.23E−04 | 6.37E−09 |
| | cPTK7-His | 8.86E+04 | 4.08E−04 | 4.61E−09 |
| | dPTK7-His | — | — | — |
| 1A7 | hPTK7-His | 1.04E+05 | 4.25E−03 | 4.09E−08 |
| | cPTK7-His | 4.11E+04 | 7.33E−04 | 1.79E−08 |
| | dPTK7-His | 5.80E+04 | 1.33E−04 | 2.30E−09 |
| 1A11 | hPTK7-His | 7.76E+04 | 3.18E−03 | 4.10E−08 |
| | cPTK7-His | 7.12E+04 | 1.31E−03 | 1.84E−08 |
| | dPTK7-His | 8.46E+04 | 1.23E−03 | 1.45E−08 |
| 1F2 | hPTK7-His | 1.72E+05 | 1.20E−02 | 6.98E−08 |
| | cPTK7-His | 3.14E+05 | 9.49E−03 | 3.02E−08 |
| | dPTK7-His | 4.37E+05 | 9.27E−03 | 2.12E−08 |
| 2A5 | hPTK7-His | 1.08E+05 | 2.81E−03 | 2.59E−08 |
| | cPTK7-His | 4.07E+04 | 4.39E−04 | 1.08E−08 |
| | dPTK7-His | 4.86E+04 | 1.67E−04 | 3.43E−09 |
| 2D11 | hPTK7-His | 3.06E+04 | 2.49E−04 | 8.11E−09 |
| | cPTK7-His | 5.74E+04 | 1.77E−04 | 3.08E−09 |
| | dPTK7-His | 6.09E+04 | 5.47E−05 | 8.98E−10 |
| 2F5 | hPTK7-His | 3.56E+04 | 3.88E−03 | 1.09E−07 |
| | cPTK7-His | 4.16E+04 | 1.46E−03 | 3.52E−08 |
| | dPTK7-His | 5.07E+04 | 5.63E−04 | 1.11E−08 |
| 3C4 | hPTK7-His | 9.32E+04 | 2.37E−03 | 2.54E−08 |
| | cPTK7-His | 8.06E+04 | 1.87E−03 | 2.32E−08 |
| | dPTK7-His | 4.71E+05 | 3.99E−03 | 8.48E−09 |
| 3E6 | hPTK7-His | 1.32E+05 | 4.54E−04 | 3.44E−09 |
| | cPTK7-His | 1.22E+05 | 4.81E−04 | 3.95E−09 |
| | dPTK7-His | 1.47E+05 | 1.71E−03 | 1.16E−08 |

(“—” means no binding)

hu24 is a humanized IgG1 monoclonal antibody targeting PTK7, and its VH and VL sequences are shown in SEQ ID NO: 77 and SEQ ID NO: 78, respectively.

The results showed that the anti-PTK7 antibodies 1A7, 1A11, 1F2, 2A5, 2D11, 2F5, 3C4 and 3E6 had good binding affinities to human PTK7, monkey PTK7 and dog PTK7.

### Binding Activity of Anti-PTK7 Antibodies

The binding activity of anti-PTK7 antibodies to cancer cell lines was verified by flow cytometry. Briefly, A-431 cells (human epidermoid carcinoma, ATCC, Cat #: CRL-1555), NCI-H520 cells (human lung cancer, ATCC, Cat #: HTB-182), NCI-N87 cells (human gastric carcinoma, ATCC, Cat #: CRL-5822), SK-OV-3 cells (human ovary cancer, ATCC, Cat #: HTB-77), BT-20 cells (human breast cancer, ATCC, Cat #: HTB-19), HCC1954 cells (human breast ductal carcinoma, ATCC, Cat #: CRL-2338), NCI-H226 cells (human lung cancer, ATCC, Cat #: CRL-5826), NCI-H358 cells (human lung cancer, ATCC, Cat #: CRL-5807), HCT116 cells (human colorectal cancer, ATCC, Cat #: CCL-247), PC-3 cells (human prostate cancer, ATCC, Cat #: CRL-1435), NCI-H292 cells (human lung cancer, ATCC, Cat #: CRL-1848), CMT-U27 cells (dog breast cancer, ATCC, Cat #: CRL-3456) and D-17 cells (dog osteosarcoma, ATCC, Cat #: CCL-183) were plated in a 96-well plate at a density of 2×10⁵ cells/well respectively. Purified anti-PTK7 antibody with a concentration of 5 μg/mL was added to each well and was incubated at 4° C. for 30 minutes. Then, after one wash with PBS, the cells were incubated with the secondary antibody Alexa Fluor® 647 anti-human IgG Fcγ (Jackson ImmunoResearch Laboratories, Inc., Cat #: 109-606-170) at 4° C. for 15 minutes before flow cytometry analysis. The cells were collected, and the mean fluorescence intensity (MFI) was determined. Human IgG1 was used as ISO control. The results are shown in the table below.

TABLE 2

| | | | | | | | | | | Hu24 |
|---|---|---|---|---|---|---|---|---|---|---|
| Cells | 1A7 | 1A11 | 1F2 | 2A5 | 2D11 | 2F5 | 3C4 | 3E6 | ISO | analog |
| A-431 | 91104 | 247047 | 155559 | 172933 | 294662 | 105899 | 185834 | 288859 | 4631 | 273181 |
| NCI-H520 | 190170 | 296401 | 215732 | 283395 | 312041 | 198610 | 281805 | 309767 | 2419 | 302352 |
| NCI-N87 | 75749 | 157576 | 107955 | 152406 | 217478 | 69435 | 139492 | 179645 | 2853 | 174384 |
| SK-OV-3 | 196638 | 319032 | 210133 | 283142 | 370736 | 177831 | 303512 | 373377 | 3859 | 331691 |
| BT-20 | 86226 | 135582 | 50855 | 137426 | 171641 | 83767 | 148165 | 151887 | 5918 | 159218 |
| HCC1954 | 118008 | 172328 | 99051 | 179345 | 210839 | 120628 | 186751 | 206992 | 6339 | 191433 |
| NCI-H226 | 80170 | 152156 | 94936 | 141300 | 180066 | 80946 | 166757 | 193219 | 9842 | 184138 |
| NCI-H358 | 52921 | 89425 | 33869 | 87702 | 103335 | 51917 | 98844 | 116986 | 3359 | 106279 |
| HCT116 | 115142 | 260470 | 148618 | 234188 | 286626 | 132976 | 230109 | 338961 | 3112 | 273707 |
| PC-3 | 32531 | 67185 | 34924 | 60736 | 72972 | 32380 | 66706 | 94615 | 3694 | 78285 |
| NCI-H292 | 43476 | 124903 | 69260 | 91984 | 134009 | 44507 | 92429 | 141445 | 2234 | 119697 |
| CMT-U27 | 56716 | 75566 | 45815 | 76363 | 72205 | 81328 | 37399 | 94534 | 1649 | 1937 |
| D-17 | 120350 | 90646 | 64106 | 95866 | 106105 | 131353 | 34628 | 86313 | 3524 | 3688 |

The results showed that all seven antibodies 1A7, 1A11, 1F2, 2A5, 2D11, 2F5, 3C4 and 3E6 exhibited binding activities to various cancer cell lines, while the positive control hu24 analog didn't bind to the dog cancer cells. In particular, the binding of 2D11 and 3E6 to the cell lines is higher than that of the positive control.

### Internalization Detection of Anti-PTK7 Antibodies

Anti-PTK7 antibodies (2.5 μg/mL) and pHAb-Goat Anti-Human IgG Secondary Antibody were added to the MDA-MB-468 cells (human breast cancer cells, Shanghai Institutes for Biological Sciences), NCI-N87 cells, CMT-U27 cells or D-17 cells. After incubating for 8 hours, the cells were centrifuged and washed in FACS buffer. MFI was detected on a flow cytometer, and the endocytosis rates of anti-PTK7 antibodies were calculated. For ISO control, human IgG1 was used.

TABLE 3

| | MDA-MB-468 | | NCI-N87 | | CMT-U27 | | D-17 | |
|---|---|---|---|---|---|---|---|---|
| | | Posi- | | Posi- | | Posi- | | Posi- |
| | | tive | | tive | | tive | | tive |
| Anti- | | cells | | cells | | cells | | cells |
| body | MFI | (%) | MFI | (%) | MFI | (%) | MFI | (%) |
| ISO | 10613 | 4.1 | 9608 | 6.4 | 9781 | 7.9 | 12384 | 6.5 |
| 1A7 | 19785 | 53.9 | 9082 | 36.9 | 8551 | 5.1 | 13844 | 9.1 |
| 1A11 | 11431 | 7.3 | 5807 | 7.6 | 6655 | 1.9 | 10067 | 3.8 |
| 1F2 | 10730 | 7.6 | 7274 | 11.6 | 8932 | 5.6 | 12199 | 7.4 |
| 2A5 | 29247 | 64.2 | 10802 | 34.8 | 13924 | 16.3 | 14913 | 14.1 |
| 2D11 | 14831 | 27.1 | 7435 | 15.2 | 7516 | 3.2 | 12593 | 6.0 |
| 2F5 | 23977 | 70.6 | 12001 | 44.0 | 10834 | 10.7 | 21650 | 17.0 |
| 3C4 | 19350 | 65.4 | 8064 | 31.9 | 7544 | 4.5 | 14011 | 15.8 |
| 3E6 | 14708 | 38.0 | 6936 | 16.5 | 7345 | 3.0 | 13244 | 10.8 |

All the anti-PTK7 antibodies exhibited good cell endocytosis rates in both MDA-MB-468 cells and NCI-N87 cells. In addition, 2A5, 2F5, 3C4 and 3E6 also had good endocytosis rates in CMT-U27 cells or D-17 cells.

### Epitope Analysis of Anti-PTK7 Antibodies

Relative positions of target protein epitope between a pair of purified anti-PTK7 antibodies were analyzed by Biolayer Interferometry (BLI) using ForteBio Octet system at 30° C. A total of seven purified antibodies were used: 1A7, 1A11, 1F2, 2A5, 2D11, 2F5, 3C4 and 3E6.

1×HBS-EP+ buffer (10 mM 4-(2-hydroxyethyl)-1-piperazineethanesulfonic acid (HEPES), 150 mM NaCl, 3 mM ethylenediaminetetraacetic acid (EDTA) and 0.05% P20, pH7.4) diluted from HBS-EP+ buffer (10×) was used as the running buffer throughout the experiment. His-tagged human PTK7 protein was captured at a flow rate of 10 μL/min, and 200 nM of antibody (Analyte 1) was injected at a flow rate of 30 μL/min to bind the ligand. Another antibody (Analyte 2) was injected under the same conditions to determine whether the binding of different antibodies interfered with each other. The binding time was 300 s for each antibody.

The binding value of each antibody was obtained using Data Analysis HT 12.0. To quantify the interference of one antibody binding to another, a binding ratio was calculated to compare each pair of antibodies. The binding ratio is defined as the binding value of the second antibody (Analyte 2), divided by the binding value of the first antibody (Analyte 1). The binding ratio of each antibody pair was summarized in a matrix table as shown in Table 4. More specifically, the binding ratio was between-0.1 to 0.4, if analyte 1 exhibited a blocking effect to analyte 2. The binding ratio was between 0.5-1.2, if analyte 1 did not exhibit a blocking effect to analyte 2. In general, antibody pairs that interfere with each other have the same or overlapping epitopes.

Accordingly, epitope correlation was analyzed and the seven human anti-PTK7 antibodies were categorized into three epitope clusters. In summary, 1A7, 2A5, 2D11, 2F5 and hu24 analog shared the same or overlapping epitopes; 3C4 and 3E6 shared the same or overlapping epitopes; 1A11 and 1F2 did not exhibit epitope correlation with the other six antibodies.

TABLE 4

| Antibody | 1A7 | 1A11 | 1F2 | 2A5 | 2D11 | 2F5 | 3C4 | 3E6 | hu24 analog |
|---|---|---|---|---|---|---|---|---|---|
| 1A7 | 0.08 | 0.74 | 0.84 | 0.10 | 0.10 | 0.07 | 0.66 | 1.05 | 0.20 |
| 1A11 | 0.71 | −0.01 | 0.86 | 0.75 | 0.71 | 0.67 | 0.67 | 0.94 | 0.97 |
| 1F2 | 0.64 | 0.64 | 0.03 | 0.71 | 0.62 | 0.56 | 0.48 | 0.75 | 0.95 |
| 2A5 | −0.02 | 0.62 | 0.78 | 0.05 | −0.07 | −0.02 | 0.62 | 0.94 | 0.04 |
| 2D11 | 0.17 | 0.83 | 0.93 | 0.15 | 0.15 | 0.14 | 0.81 | 1.18 | 0.21 |
| 2F5 | 0.12 | 0.71 | 0.85 | 0.12 | 0.07 | 0.08 | 0.71 | 1.02 | 0.19 |
| 3C4 | 0.87 | 0.84 | 0.90 | 0.93 | 0.91 | 0.86 | 0.16 | 0.17 | 1.22 |
| 3E6 | 0.71 | 0.71 | 0.74 | 0.81 | 0.76 | 0.71 | 0.11 | 0.08 | 0.98 |
| hu24 | 0.11 | 0.75 | 0.83 | 0.11 | 0.14 | 0.12 | 0.68 | 1.05 | 0.13 |
| analog | | | | | | | | | |

### Example 2. Anti-TROP2 Antibody

### Generating Anti-TROP2 Antibodies

RenMice (i.e., an engineered mouse comprising DNA encoding human immunoglobulin heavy and kappa light chain variable regions, for example, RenMab™ mice, RenLite™ mice) were immunized with Fc-tagged human TROP2 protein (ACRO Biosystems Inc., Cat #: TR2-H5253) and his-tagged canine TROP2 protein (ACRO Biosystems Inc., Cat #: TR2-C52H4) to generate antibodies against TROP2.

The antibody immune response was monitored by an antigen-specific immunoassay. When a desired immune response was achieved, antigen-specific immune cells were isolated from the immunized mice to further obtain the light chain and heavy chain variable region sequences of the anti-TROP2 antibodies. The obtained variable region sequences were cloned into a vector containing a sequence encoding the human IgG1 constant region for antibody expression. Exemplary antibodies obtained included 18E9 and 18F12.

These antibodies contained substantially the same light chain, and their VH and VL CDR 1-3 sequences are shown in FIG. 1B and FIG. 2B. The VH and the VL regions of 18E9 and 18F12 are shown in FIG. 3.

### Binding Affinity of Anti-TROP2 Antibodies

The affinity of the anti-TROP2 antibodies to His-tagged human TROP2 protein (hTROP2-His, ACRO Biosystems Inc., Cat #: TR2-H5223), His-tagged cynomolgus monkey TROP2 protein (cTROP2-His, ACRO Biosystems Inc., Cat #: TR2-R52H3) and His-tagged dog TROP2 protein (dTROP2-His, ACRO Biosystems Inc., Cat #: TR2-C52H4) were measured by surface plasmon resonance (SPR) using Biacore™ (Biacore, INC, Piscataway N.J.) 8K biosensor equipped with pre-immobilized Protein A sensor chips.

Purified anti-TROP2 antibodies were diluted to 1 μg/mL and then injected into the Biacore™ 8K biosensor at 10 μL/min for about 50 seconds to achieve a desired protein density (e.g., about 350 response units (RU)). The His-tagged TROP2 protein at a concentration of 200 nM was then injected at 30 μL/min for 180 seconds. Dissociation was monitored for 400 seconds. The chip was regenerated after the last injection of each titration with glycine (pH 2.0, 30 μL/min for 30 seconds).

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

The results for the tested antibodies are summarized in the table below.

TABLE 5

| Antibody | Protein | kon (1/Ms) | koff (1/s) | KD (M) |
|---|---|---|---|---|
| DS-1062 analog- | hTROP2-His | 1.11E+05 | 4.11E−03 | 3.70E−08 |
| LALA | cTROP2-His | 1.26E+05 | 4.09E−03 | 3.25E−08 |
| | dTROP2-His | 2.99E+04 | 8.12E−04 | 2.72E−08 |
| Sacituzumab | hTROP2-His | 2.05E+05 | 2.55E−04 | 1.24E−09 |
| analog | cTROP2-His | 2.21E+05 | 2.45E−04 | 1.11E−09 |
| | dTROP2-His | 2.04E+05 | 8.58E−03 | 4.20E−08 |
| 18E9 | hTROP2-His | 7.98E+05 | 1.17E−03 | 1.46E−09 |
| | cTROP2-His | 1.10E+06 | 1.32E−03 | 1.20E−09 |
| | dTROP2-His | 2.23E+05 | 1.08E−03 | 4.84E−09 |
| 18F12 | hTROP2-His | 7.13E+05 | 5.11E−03 | 7.16E−09 |
| | cTROP2-His | 8.03E+05 | 4.35E−03 | 5.41E−09 |
| | dTROP2-His | 1.18E+05 | 2.55E−03 | 2.16E−08 |

Sacituzumab govitecan (Trodelvy™) is a humanized anti-TROP2 monoclonal antibody-drug conjugate. The heavy chain variable region and light chain variable region of Sacituzumab govitecan are shown as SEQ ID NOs: 79-80.

DS-1062 is a TROP2-directed antibody drug conjugate, which is in phase III clinical trials in patients with advanced or metastatic non-small cell lung cancer (NSCLC). The heavy chain variable region and light chain variable region of DS-1062 are shown as SEQ ID NOs: 81-82.

The antibody portion of Sacituzumab govitecan and DS-1062 were used for comparison purpose, and were named as Sacituzumab analog and DS-1062 analog respectively. The constant region of the antibodies can include some mutations (e.g., LALA mutations, SI mutations). When LALA mutations are introduced into the Fc region of DS-1062 analog, the resulting antibody is named as DS-1062 analog-LALA. When SI mutations are introduced into the Fc region of DS-1062 analog, the resulting antibody is named as DS-1062-SI.

### Internalization Detection of Anti-TROP2 Antibodies

Anti-TROP2 antibodies (2.5 μg/mL) and pHAb-Goat Anti-Human IgG Secondary Antibody were added to the NCI-H292 cells, NCI-H1650 cells and CMT-U27 cells. After incubating for 1 hours, the cells were centrifuged and washed in FACS buffer. MFI was detected on a flow cytometer. The results are shown in the table below.

TABLE 6

| | NCI-H292 | | NCI-H1650 | | CMT-U27 | |
|---|---|---|---|---|---|---|
| | | Positive | | Positive | | Positive |
| Anti- | | cells | | cells | | cells |
| body | MFI | (%) | MFI | (%) | MFI | (%) |
| ISO | 5951.12 | 4.25 | 11817.47 | 8.73 | 5801.99 | 0.99 |
| DS- | 32987.31 | 99.83 | 20796.54 | 41.33 | 6169.29 | 3.18 |
| 1062 | | | | | | |
| ana- | | | | | | |
| log- | | | | | | |
| LALA | | | | | | |
| 18E9 | 57525.61 | 100 | 32441.42 | 80.88 | 10832.53 | 56.49 |
| 18F12 | 72671.60 | 99.97 | 28031.99 | 68.63 | 12140.00 | 66.29 |

The results showed that anti-TROP2 antibodies 18E9 and 18F12 exhibited good endocytosis rates in NCI-H292 cells, NCI-H1650 cells and CMT-U27 cells.

### Example 3. Anti-PTK7/TROP2 Bispecific Antibodies

### Generating Anti-PTK7/TROP2 Bispecific Antibodies

Above anti-PTK7 antibodies and anti-TROP2 antibodies can be paired to form various bispecific antibodies. Vectors encoding the light chain and heavy chain of the antibodies were constructed. CHO-S cells were co-transfected with three vectors, including a first vector encoding the heavy chain of an anti-PTK7 antibody, a second vector encoding the heavy chain of anti-TROP2 antibody, and a third vector encoding the common light chain. After 14 days of culture, the cell supernatant was collected and purified by Protein A affinity chromatography.

6F7 is an anti-TROP2 monoclonal antibody developed by Biocytogen Pharmaceuticals (Beijing) Co., Ltd., the VH CDR 1-3 sequences are shown in FIG. 1B and FIG. 2B. The VH region of 6F7 is shown in FIG. 3. Details related to 6F7 is described in PCT/CN2023/073039, which is incorporated herein by reference in its entirety.

Knobs-into-holes mutations were introduced in the Fc regions of the antibodies to reduce the chance of wrong pairing between the two heavy chains. Exemplary bispecific antibodies obtained include: 3C4-6F7, 3C4-18E9, 3C4-18F12, 3E6-6F7, 3E6-18E9 and 3E6-18F12. In 3C4-6F7, the heavy chain constant region of 3C4 includes knob mutations, and the heavy chain constant region of 6F7 includes hole mutations. The sequence of the light chain constant region is set forth in SEQ ID NO: 83; the human IgG1 constant region with knob mutations is set forth in SEQ ID NO: 84; and the human IgG1 constant region with hole mutations is set forth in SEQ ID NO: 85.

### Binding Activity of Anti-PTK7/TROP2 Bispecific Antibodies

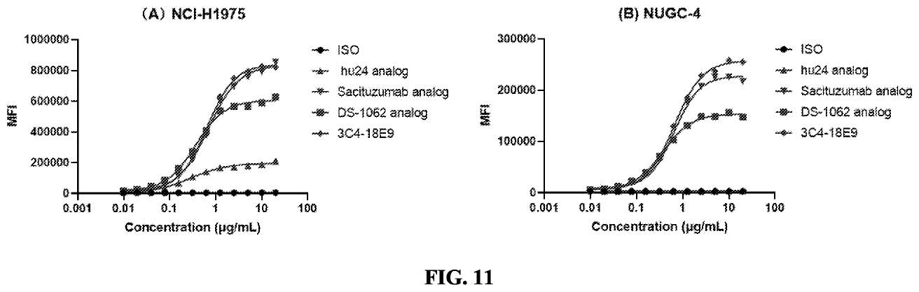

This experiment was performed to test the binding activities of the anti-PTK7/TROP2 bispecific antibodies to different tumor cell lines. NCI-H1975 cells or NUGC-4 cells were transferred to a 96-well plate at a density of 5×10⁴ cells/well respectively. The sample antibody was added to the 96-well plate, and incubated at 4° C. for 30 min. Then, the cells were incubated with the secondary antibody anti-hIgG-Fc-Alex Flour 647 (RL1-H) (Jackson ImmunoResearch Laboratories, Inc., 109-606-170) at 4° C. in the dark for 15 minutes before flow cytometry analysis. The results are shown in FIG. 11, in which 3C4-18E9 showed good binding in NCI-H1975 cells with PTK7/TROP2 co-expression (FIG. 11(A)) and NUGC-4 cells with only TROP2 expression (FIG. 11(B)).

### Internalization Detection of Anti-PTK7/TROP2 Bispecific Antibodies

Anti-PTK7/TROP2 bispecific antibodies (2.5 μg/mL) and pHAb-Goat Anti-Human IgG Secondary Antibody were added to the MDA-MB-468 cells, A-431 cells, NCI-H292 cells or NUGC-4 cells. After incubating for 2-6 hours, the cells were centrifuged and washed in FACS buffer. MFI was detected on a flow cytometer, and the endocytosis rates of anti-PTK7 antibodies were calculated.

TABLE 7

| | MDA-MB- | | | | | | | |
|---|---|---|---|---|---|---|---|---|
| | 468 | | A-431 | | NCI-H292 | | NUGC-4 | |
| | (2 hours) | | (6 hours) | | (2 hours) | | (2 hours) | |
| | | Posi- | | Posi- | | Posi- | | Posi- |
| | | tive | | tive | | tive | | tive |
| Anti- | | cells | | cells | | cells | | cells |
| body | MFI | (%) | MFI | (%) | MFI | (%) | MFI | (%) |
| ISO | 7746 | 1.8 | 10637 | 29.9 | 6052 | 2.1 | 5210 | 0.4 |
| hu24 | 10390 | 7.8 | 25826 | 85.4 | 8763 | 16.3 | 5180 | 0.6 |
| ana- | | | | | | | | |
| log | | | | | | | | |
| 3C4- | 15767 | 73.4 | 77694 | 93.6 | 24568 | 99.0 | 6035 | 3.2 |
| 6F7 | | | | | | | | |
| 3C4- | 21400 | 96.9 | 268336 | 99.0 | 48705 | 99.6 | 8840 | 21.6 |
| 18E9 | | | | | | | | |
| 3C4- | 15266 | 67.6 | 146525 | 99.1 | 25524 | 99.2 | 6948 | 5.4 |
| 18F12 | | | | | | | | |
| 3E6- | 13259 | 44.0 | 67083 | 98.2 | 15583 | 92.9 | 5509 | 0.9 |
| 6F7 | | | | | | | | |
| 3E6- | 40478 | 99.6 | 273910 | 96.7 | 109555 | 100 | 13259 | 54.0 |
| 18E9 | | | | | | | | |
| 3E6- | 30443 | 99.5 | 238592 | 96.5 | 69694 | 99.8 | 11999 | 47.4 |
| 18F12 | | | | | | | | |

The data showed that the anti-PTK7/TROP2 antibodies exhibited good cell endocytosis rates compared with the positive control hu24 analog in MDA-MB-468 cells, A-431 cells, NCI-H292 cells and NUGC-4 cells.

In a similar experiment, antibodies (2.5 μg/mL) and pHAb-Goat Anti-Human IgG Secondary Antibody were added to the BT-20 cells or HCC70 cells. After incubating for 5 hours, the cells were centrifuged and washed in FACS buffer. MFI was detected on a flow cytometer, and the endocytosis rates of anti-PTK7 antibodies were calculated. The results are shown in the table below. The anti-PTK7/TROP2 antibodies 3C4-6F7, 3E6-6F7, 3C4-18E9 and 3E6-18E9 showed increased endocytosis activities compared with their parental monoclonal antibodies 3C4 or 3E6.

TABLE 8

| | BT-20 | | HCC70 | |
|---|---|---|---|---|
| Antibody | MFI | Positive cells (%) | MFI | Positive cells (%) |
| ISO | 9458 | 5.63 | 8763 | 4.13 |
| 3C4-6F7 | 44336 | 89.1 | 52570 | 99.1 |
| 3E6-6F7 | 59822 | 94.9 | 73477 | 99.3 |
| 3C4-18E9 | 147388 | 99.6 | 186427 | 99.8 |
| 3E6-18E9 | 165276 | 97.9 | 204198 | 99.1 |
| 3C4 | 15446 | 13.7 | 24713 | 86.0 |
| 3E6 | 18154 | 21.0 | 25902 | 92.5 |

### Binding Affinity of Anti-PTK7 Bispecific Antibodies

The affinity of the anti-PTK7/TROP2 bispecific antibodies to His-tagged human TROP2 protein (hTROP2-His, ACRO Biosystems Inc., Cat #: TR2-H5223), His-tagged Rhesus macaque monkey TROP2 protein (cTROP2-His, ACRO Biosystems Inc., Cat #: Cat: TR2-R52H3), His-tagged dog TROP2 protein (dTROP2-His, ACRO Biosystems Inc., Cat #: TR2-C52H4), hPTK7-His, cPTK7-His and dPTK7-His were measured by surface plasmon resonance (SPR) using Biacore™ (Biacore, INC, Piscataway N.J.) 8K biosensor equipped with pre-immobilized Protein A sensor chips. The results are summarized in the table below.

TABLE 9

| Antibody | Protein | kon (1/Ms) | koff (1/s) | KD (M) |
|---|---|---|---|---|
| 3C4-6F7 | hPTK7-His | 1.15E+05 | 1.52E−03 | 1.32E−08 |
| | cPTK7-His | 1.24E+05 | 2.16E−03 | 1.74E−08 |
| | dPTK7-His | 1.14E+05 | 6.00E−03 | 5.25E−08 |
| | hTROP2-His | 3.94E+05 | 4.19E−03 | 1.06E−08 |
| | cTROP2-His | 3.01E+05 | 1.79E−03 | 5.94E−09 |
| | dTROP2-His | — | — | — |
| 3C4-18E9 | hPTK7-His | 1.28E+05 | 1.25E−03 | 9.79E−09 |
| | cPTK7-His | 1.27E+05 | 1.67E−03 | 1.31E−08 |
| | dPTK7-His | 1.15E+05 | 4.49E−03 | 3.89E−08 |
| | hTROP2-His | 3.53E+05 | 1.07E−03 | 3.03E−09 |
| | cTROP2-His | 3.29E+05 | 1.04E−03 | 3.16E−09 |
| | dTROP2-His | 3.17E+05 | 1.64E−02 | 5.18E−08 |
| 3C4-18F12 | hPTK7-His | 1.21E+05 | 1.39E−03 | 1.15E−08 |
| | cPTK7-His | 1.17E+05 | 1.73E−03 | 1.48E−08 |
| | dPTK7-His | 1.18E+05 | 4.26E−03 | 3.62E−08 |
| | hTROP2-His | 2.24E+05 | 2.50E−03 | 1.12E−08 |
| | cTROP2-His | 2.09E+05 | 2.52E−03 | 1.21E−08 |
| | dTROP2-His | — | — | — |
| 3E6-6F7 | hPTK7-His | 1.38E+05 | 3.69E−04 | 2.67E−09 |
| | cPTK7-His | 1.14E+05 | 5.22E−04 | 4.60E−09 |
| | dPTK7-His | 9.55E+04 | 1.97E−03 | 2.06E−08 |
| | hTROP2-His | 1.30E+05 | 4.55E−02 | 3.51E−07 |
| | cTROP2-His | 3.52E+05 | 2.84E−03 | 8.07E−09 |
| | dTROP2-His | — | — | — |
| 3E6-18E9 | hPTK7-His | 1.24E+05 | 3.89E−04 | 3.14E−09 |
| | cPTK7-His | 1.20E+05 | 6.23E−04 | 5.20E−09 |
| | dPTK7-His | 9.83E+04 | 1.64E−03 | 1.67E−08 |
| | hTROP2-His | 3.60E+05 | 1.16E−03 | 3.23E−09 |
| | cTROP2-His | 3.25E+05 | 1.19E−03 | 3.66E−09 |
| | dTROP2-His | 2.65E+05 | 1.93E−02 | 7.30E−08 |
| 3E6-18F12 | hPTK7-His | 1.35E+05 | 3.29E−04 | 2.43E−09 |
| | cPTK7-His | 1.30E+05 | 6.49E−04 | 4.98E−09 |
| | dPTK7-His | 1.05E+05 | 1.72E−03 | 1.64E−08 |
| | hTROP2-His | 1.78E+05 | 2.44E−03 | 1.37E−08 |
| | cTROP2-His | 1.66E+05 | 2.31E−03 | 1.39E−08 |
| | dTROP2-His | — | — | — |

(“—” means no binding)

### Example 4. Generating Antibody Drug Conjugates (ADC)

Each purified antibody (3C4, 3E6, 6F7, 18E9, 3C4-6F7, 3C4-18E9, 3C4-18F12, 3E6-6F7, 3E6-18E9 or 3E6-18F12) was coupled with MMAE (monomethyl auristatin E) through a maleimidocaproyl-valine-citrulline-p-aminobenzyloxycarbonyl (VC) linker.

For the names of antibody drug conjugates, “ADC” is added directly after the antibody name. For example, if 3C4 is coupled to MMAE, it is named as 3C4-ADC. If 3C4-6F7 is coupled to MMAE, it is named as 3C4-6F7-ADC. Human IgG1 and hu24 were coupled to MMAE for the isotype control and positive control, and named as ISO-ADC and hu24-ADC, respectively. For reference purpose, DS-1062 analog was coupled to Dxd through GGFG linker to form DS-1062-Dxd.

HIC-HPLC was performed to detect the coupling of antibodies with drug molecules. The results show that the drug-to-antibody ratio (DAR) of the ADCs is about 4.

### Example 5. In Vitro Killing Activity

Purified ADCs with different concentrations (10 μg/mL, 3.333 μg/mL, 1.111 μg/mL, 0.370 μg/mL, 0.123 μg/mL, 0.041 μg/mL, 0.014 μg/mL, 0.005 μg/mL, 0.002 μg/mL, 0.001 μg/mL,) were used to treat MDA-MB-468 cells or HCC70 cells cultured in a cell culture plate, and the killing activity was detected after 72 hours of incubation with IncuCyte (Sartorius AG, IncuCyte® S3). The results are shown in the table below.

TABLE 10

IC50 (μg/mL)

| ADC | MDA-MB-468 | HCC70 |
|---|---|---|
| 3C4-6F7-ADC | 0.3146 | 0.2522 |
| 3E6-6F7-ADC | 0.1438 | 0.1082 |
| 3C4-ADC | 1.4790 | 3.083 |
| 3E6-ADC | 1.7800 | 2.945 |

The datas showed that 3C4-6F7-ADC and 3E6-6F7-ADC exhibited better internalization activities than that of the corresponding parental monoclonal antibody ADC (3C4-ADC or 3E6-ADC).

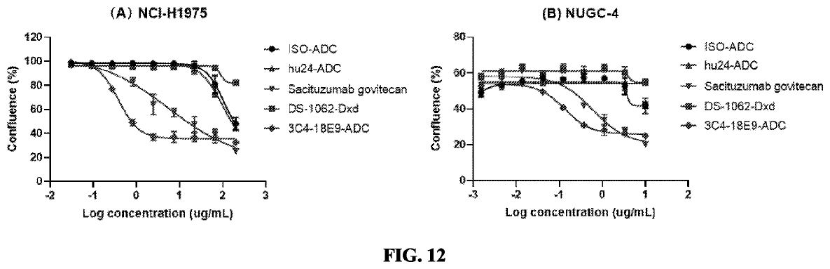

In another similar experiment, the killing activity of 3C4-18E9-ADC in lung cancer cells NCI-H1975 or gastric cancer cells NUGC-4 was tested. Human IgG1 was used as an ISO control. The results were shown in FIG. 12, in which 3C4-18E9-ADC showed potent cytotoxicity in both NCI-H1975 cells (FIG. 12(A)) and NUGC-4 cells (FIG. 12(B)) compared with the positive controls hu24-ADC, Sacituzumab govitecan and DS-1062-Dxd.

### Example 6. Anti-Tumor Activity in MDA-MB-468 Xenograft Model

About 1×10⁷ MDA-MB-468 cells were injected subcutaneously in each B-NDG mouse (Biocytogen Pharmaceuticals (Beijing) Co., Ltd., Cat #: B-CM-002). When the tumors in the mice reached a volume of about 200 mm³, the mice were randomly placed into different groups based on tumor size. The mice were then injected with phosphate buffer saline (PBS) or anti-PTK7 ADCs by intravenosus (i.v.) administration on the day of group assignment (Day 0). Details are shown in the table below.

TABLE 11

| | | | | | | Total |
|---|---|---|---|---|---|---|
| | No. | | | | | No. of |
| | of | | Dos- | | Fre- | admin- |
| Group | mice | ADC | age | Route | quency | istration |
| G1 | 5 | PBS | — | i.v. | Day 0, | 2 |
| | | | | | Day 31 | |
| G2 | 5 | hu24-ADC | 3 | i.v. | Day 0, | 2 |
| | | | mg/ | | Day 31 | |
| | | | kg | | | |
| G3 | 5 | Sacituzumab | 3 | i.v. | Day 0, | 2 |
| | | govitecan | mg/ | | Day 31 | |
| | | | kg | | | |
| G4 | 5 | 3C4-18E9- | 3 | i.v. | Day 0, | 2 |
| | | ADC | mg/ | | Day 31 | |
| | | | kg | | | |
| G5 | 5 | 3C4-18F12- | 3 | i.v. | Day 0, | 2 |
| | | ADC | mg/ | | Day 31 | |
| | | | kg | | | |
| G6 | 5 | 3C4-6F7- | 3 | i.v. | Day 0, | 2 |
| | | ADC | mg/ | | Day 31 | |
| | | | kg | | | |
| G7 | 5 | 3E6-18E9- | 3 | i.v. | Day 0, | 2 |
| | | ADC | mg/ | | Day 31 | |
| | | | kg | | | |
| G8 | 5 | 3E6-18F12- | 3 | i.v. | Day 0, | 2 |
| | | ADC | mg/ | | Day 31 | |
| | | | kg | | | |
| G9 | 5 | 3E6-6F7- | 3 | i.v. | Day 0, | 2 |
| | | ADC | mg/ | | Day 31 | |
| | | | kg | | | |
| G10 | 5 | 3C4-ADC | 3 | i.v. | Day 0, | 2 |
| | | | mg/ | | Day 31 | |
| | | | kg | | | |
| G11 | 5 | 3E6-ADC | 3 | i.v. | Day 0, | 2 |
| | | | mg/ | | Day 31 | |
| | | | kg | | | |
| G12 | 5 | 18E9-ADC | 3 | i.v. | Day 0, | 2 |
| | | | mg/ | | Day 31 | |
| | | | kg | | | |

The length of the long axis and the short axis of the tumor were measured and the volume of the tumor was calculated as 0.5×(long axis)×(short axis) 2. The tumor growth inhibition percentage (TGI %) was calculated using the following formula: TGI (%)=[1−(Tᵢ−T₀)/(Vᵢ−V₀)]×100%. Tᵢ is the average tumor volume in the treatment group on day i. T₀ is the average tumor volume in the treatment group on Day 0. Vᵢ is the average tumor volume in the control group on Day i. V₀ is the average tumor volume in the control group on Day 0. Values are expressed as mean±SEM. T-test was performed for statistical analysis. P<0.05 is a threshold to indicate a significant difference.

The weights of mice in different groups all increased. On the day of group assignment (Day 0), the average weight of each group was in the range of 21.6 g-22.1 g. 87 days post grouping (Day 87), the average body weight of each group was in the range of 23.8 g-25.7 g and the average body weight change of each group was in the range of 108.6%-118.9%. The results showed that these tested ADCs were well tolerated and were not obviously toxic to the mice.

The table below summarizes the results for this experiment, including the tumor volumes on the day of grouping (Day 0), 20 days after grouping (Day 20), 41 days after grouping (Day 41), 62 days after grouping (Day 62) and 87 days after grouping (Day 87); TGI; and the statistical differences (P value) of tumor volume and body weight between the treatment and control groups on Day 87.

TABLE 12

| | | | | | | | P value | |
|---|---|---|---|---|---|---|---|---|
| | Tumor volume (mm³) | | | | | TGI | Tumor | Body |
| Group | Day 0 | Day 20 | Day 41 | Day 62 | Day 87 | (%) | volume | weight |
| G1 | 199 ± 3 | 384 ± 26 | 817 ± 57 | 1195 ± 118 | 1772 ± 213 | NA | NA | NA |
| G2 | 199 ± 5 | 219 ± 25 | 382 ± 29 | 592 ± 85 | 1154 ± 113 | 39.3 | 0.029 | 0.427 |
| G3 | 199 ± 6 | 284 ± 26 | 561 ± 58 | 891 ± 123 | 1343 ± 185 | 27.3 | 0.171 | 0.244 |
| G4 | 199 ± 10 | 77 ± 19 | 120 ± 22 | 191 ± 32 | 442 ± 70 | 84.6 | 3.19E−04 | 0.339 |
| G5 | 199 ± 6 | 139 ± 38 | 158 ± 46 | 259 ± 89 | 563 ± 125 | 76.8 | 0.001 | 0.647 |
| G6 | 199 ± 10 | 116 ± 22 | 137 ± 25 | 192 ± 31 | 501 ± 75 | 80.8 | 4.47E−04 | 0.252 |
| G7 | 199 ± 10 | 127 ± 27 | 170 ± 34 | 269 ± 71 | 652 ± 159 | 71.2 | 0.004 | 0.844 |
| G8 | 199 ± 7 | 115 ± 38 | 131 ± 45 | 257 ± 93 | 529 ± 159 | 79.0 | 0.002 | 0.462 |
| G9 | 199 ± 5 | 151 ± 20 | 166 ± 33 | 249 ± 31 | 555 ± 71 | 77.4 | 0.001 | 0.784 |
| G10 | 199 ± 5 | 134 ± 12 | 172 ± 10 | 364 ± 26 | 773 ± 85 | 63.5 | 0.002 | 0.804 |
| G11 | 199 ± 5 | 190 ± 30 | 249 ± 38 | 335 ± 39 | 887 ± 116 | 56.3 | 0.006 | 0.424 |
| G12 | 199 ± 7 | 259 ± 30 | 456 ± 30 | 602 ± 29 | 1100 ± 82 | 42.7 | 0.015 | 0.660 |

The tumor volume of mice in different groups treated with the ADCs or PBS are shown in FIG. 5. Compared with the positive control hu24-ADC (G2) and Sacituzumab govitecan (G3), all the six anti-PTK7/TROP2 bispecific ADCs 3C4-18E9-ADC (G4), 3C4-18F12-ADC (G5), 3C4-6F7-ADC (G6), 3E6-18E9-ADC (G7), 3E6-18E9-ADC (G8) and 3E6-6F7-ADC (G9) exhibited better tumor inhibitory effect. The corresponding anti-PTK7 ADCs 3C4-ADC (G10) and 3E6-ADC (G11) also obtained better anti-tumor activities than the positive control hu24-ADC (G2). And the corresponding anti-TROP2 ADC 18E9-ADC (G12) also obtained better anti-tumor activity than the positive control Sacituzumab govitecan (G3).

In addition, the experiment was then continued until to 108 days after grouping (Day 108), and the anti-PTK7/TROP2 bispecific antibody ADCs 3C4-18E9-ADC (G4), 3C4-18F12-ADC (G5), 3C4-6F7-ADC (G6), 3E6-18E9-ADC (G7), 3E6-18E9-ADC (G8) and 3E6-6F7-ADC (G9) still showed better tumor inhibitory effect with TGI of 65.4%, 59.2%, 65.7%, 42.4%, 66.2% and 62.9% respectively than that of the positive controls hu24-ADC with TGI of 25.8% and Sacituzumab govitecan with TGI of 14.7% respectively.

The results indicate that the anti-PTK7/TROP2 bispecific ADCs have a synergistic effect on tumor suppression.

### Example 7. Anti-Tumor Activity in Human Breast Cancer PDX Model

The ADCs were tested for the effect in human breast cancer patient-derived xenograft model. Immunofluorescence staining of patient-derived breast tumor fragment was performed and the images were analyzed via HALO 3.2 version. The results showed that the percentage of PTK7 positive cells and TROP2 positive cells in the tumor tissue were 11.32% and 88.82% respectively. B-NDG mice were engrafted in the right flank with the patient-derived tumor fragment (2 mm×2 mm×2 mm). When the tumors in the mice reached a volume of about 150 mm³, the mice were randomly placed into different groups based on the volume of the tumor. The mice were then injected with PBS or ADCs by intravenosus (i.v.) administration. Details of the administration scheme are shown in the table below.

TABLE 13

| | | | | | | Total |
|---|---|---|---|---|---|---|
| | No. | | | | | No. of |
| | of | | | | Fre- | admin- |
| Group | mice | ADC | Dosage | Route | quency | istration |
| G1 | 5 | PBS | — | i.v. | QW | 1 |
| G2 | 5 | 3C4- | 3 mg/kg | i.v. | QW | 1 |
| | | 18E9- | | | | |
| | | ADC | | | | |
| G3 | 5 | 3C4- | 3 mg/kg | i.v. | QW | 1 |
| | | 6F7- | | | | |
| | | ADC | | | | |
| G4 | 5 | 3E6- | 3 mg/kg | i.v. | QW | 1 |
| | | 18E9- | | | | |
| | | ADC | | | | |
| G5 | 5 | 3E6- | 3 mg/kg | i.v. | QW | 1 |
| | | 6F7- | | | | |
| | | ADC | | | | |
| G6 | 5 | 3C4- | 6 mg/kg | i.v. | QW | 1 |
| | | 18E9- | | | | |
| | | ADC | | | | |
| G7 | 5 | 3C4- | 6 mg/kg | i.v. | QW | 1 |
| | | 6F7- | | | | |
| | | ADC | | | | |
| G8 | 5 | 3E6- | 6 mg/kg | i.v. | QW | 1 |
| | | 18E9- | | | | |
| | | ADC | | | | |
| G9 | 5 | 3E6- | 6 mg/kg | i.v. | QW | 1 |
| | | 6F7- | | | | |
| | | ADC | | | | |

The table below summarizes the results for this experiment, including the tumor volumes on the day of grouping (Day 0), 21 days after grouping (Day 21), and 35 days after grouping (Day 35); TGI; and the statistical differences (P value) of tumor volume and body weight between the treatment and control groups on Day 35.

TABLE 14

| | | | | | | P value | |
|---|---|---|---|---|---|---|---|
| | Tumor volume (mm³) | | | Tumor- | TGI | Tumor | Body |
| Group | Day 0 | Day 21 | Day 35 | free | (%) | volume | weight |
| G1 | 139 ± 13 | 695 ± 170 | 1478 ± 464 | 0/5 | NA | NA | NA |
| G2 | 139 ± 11 | 251 ± 62 | 663 ± 117 | 0/5 | 60.9 | 0.099 | 0.332 |
| G3 | 139 ± 13 | 164 ± 87 | 541 ± 257 | 0/5 | 70.0 | 0.104 | 0.240 |
| G4 | 139 ± 14 | 243 ± 38 | 470 ± 65 | 0/5 | 75.2 | 0.045 | 0.465 |
| G5 | 138 ± 13 | 190 ± 59 | 506 ± 132 | 0/5 | 72.5 | 0.060 | 0.208 |
| G6 | 139 ± 14 | 11 ± 2 | 27 ± 10 | 1/5 | 108.4 | 0.009 | 0.194 |
| G7 | 138 ± 14 | 11 ± 3 | 41 ± 13 | 0/5 | 107.3 | 0.010 | 0.414 |
| G8 | 139 ± 18 | 23 ± 8 | 73 ± 28 | 1/5 | 104.9 | 0.011 | 0.299 |
| G9 | 139 ± 20 | 7 ± 3 | 18 ± 8 | 1/5 | 109.0 | 0.009 | 0.043 |

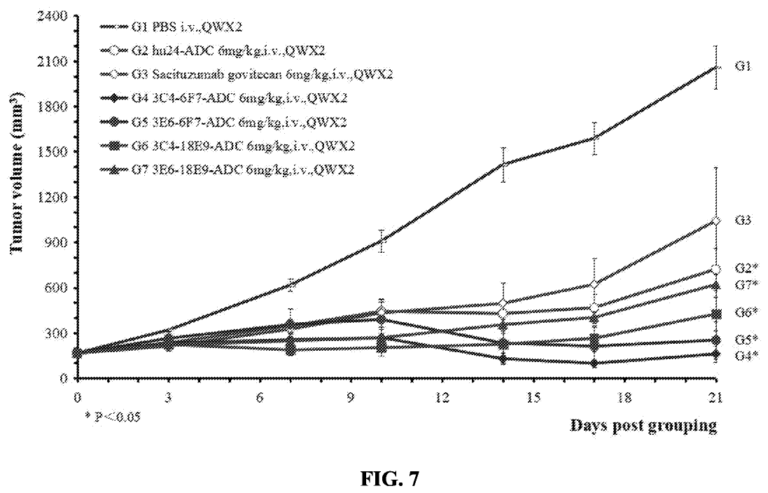

The tumor volume of mice in different groups treated with the ADCs or PBS are shown in FIG. 6. Compared with the control group (G1), all the four anti-PTK7/TROP2 bispecific ADCs 3C4-18E9-ADC, 3C4-6F7-ADC, 3E6-18E9-ADC and 3E6-6F7-ADC exhibited better tumor inhibitory effect at different dose levels, and higher doses led to better therapeutic effects. In particular, the tumor in one mouse disappeared completely in 3C4-18E9-ADC, 3E6-18E9-ADC and 3E6-6F7-ADC treatment groups on Day 35. The results indicates that anti-PTK7/TROP2 bispecific ADCs have potential tumor killing effect in breast cancer therapy.

In a similar experiment, B-NDG mice were engrafted in the right flank with another patient-derived tumor fragment (2 mm×2 mm×2 mm). The percentage of PTK7 positive cells and TROP2 positive cells in the tumor tissue were 72.16% and 91.08% respectively by immunofluorescence test. When the tumors in the mice reached a volume of about 150 mm³, the mice were randomly placed into different groups based on the volume of the tumor. The mice were then injected with PBS or ADCs by i.v. administration. Details of the administration scheme are shown in the table below.

TABLE 15

| | | | | | | Total |
|---|---|---|---|---|---|---|
| | No. | | | | | No. of |
| | of | | | | Fre- | admin- |
| Group | mice | ADC | Dosage | Route | quency | istration |
| G1 | 5 | PBS | — | i.v. | QW | 2 |
| G2 | 5 | hu24- | 5 mg/kg | i.v. | QW | 2 |
| | | ADC | | | | |
| G3 | 5 | 3C4- | 5 mg/kg | i.v. | QW | 2 |
| | | 18E9- | | | | |
| | | ADC | | | | |
| G4 | 5 | 3C4- | 5 mg/kg | i.v. | QW | 2 |
| | | 6F7- | | | | |
| | | ADC | | | | |
| G5 | 5 | 3E6- | 5 mg/kg | i.v. | QW | 2 |
| | | 18E9- | | | | |
| | | ADC | | | | |
| G6 | 5 | 3E6- | 5 mg/kg | i.v. | QW | 2 |
| | | 6F7- | | | | |
| | | ADC | | | | |
| G7 | 5 | 3C4- | 10 mg/kg | i.v. | QW | 2 |
| | | 18E9- | | | | |
| | | ADC | | | | |
| G8 | 5 | 3C4- | 10 mg/kg | i.v. | QW | 2 |
| | | 6F7- | | | | |
| | | ADC | | | | |
| G9 | 5 | 3E6- | 10 mg/kg | i.v. | QW | 2 |
| | | 18E9- | | | | |
| | | ADC | | | | |
| G10 | 5 | 3E6- | 10 mg/kg | i.v. | QW | 2 |
| | | 6F7- | | | | |
| | | ADC | | | | |

The table below summarizes the results for this experiment, including the tumor volumes on Day 0, Day 14 and Day 25; TGI; and the statistical differences (P value) of tumor volume between the treatment and control groups on Day 21. As a large number of mice in the control group (G1) died, P value on Day 25 was not available.

TABLE 16

| | Tumor volume (mm³) | | | TGI (%) | P value | Tumor-free | Survival | TGI (%) |
|---|---|---|---|---|---|---|---|---|
| Group | Day 0 | Day 14 | Day 25 | (Day 21) | (Day 21) | (Day 21) | (Day 25) | (Day 25) |
| G1 | 148 ± 15 | 1675 ± 180 | 3553 ± 0 | NA | NA | 0 | 1/5 | NA |
| G2 | 148 ± 18 | 760 ± 82 | 2790 ± 258 | 44.8 | 0.004 | 0 | 5/5 | 22.4 |
| G3 | 149 ± 17 | 133 ± 79 | 392 ± 171 | 95.9 | 1.20E−05 | 0 | 5/5 | 92.9 |
| G4 | 148 ± 15 | 367 ± 66 | 1351 ± 330 | 77.9 | 1.12E−04 | 0 | 5/5 | 64.7 |
| G5 | 148 ± 16 | 73 ± 62 | 116 ± 108 | 102.0 | 3.18E−06 | 0 | 5/5 | 100.9 |
| G6 | 148 ± 16 | 205 ± 81 | 773 ± 343 | 91.2 | 2.30E−05 | 0 | 5/5 | 81.6 |
| G7 | 148 ± 21 | 28 ± 27 | 11 ± 10 | 103.7 | 2.00E−06 | 2 | 5/5 | 104.0 |
| G8 | 148 ± 27 | 9 ± 6 | 2 ± 2 | 104.3 | 1.86E−06 | 4 | 5/5 | 104.3 |
| G9 | 148 ± 20 | 6 ± 2 | 3 ± 1 | 104.3 | 1.86E−06 | 2 | 5/5 | 104.2 |
| G10 | 148 ± 20 | 6 ± 0 | 5 ± 3 | 104.3 | 1.86E−06 | 2 | 5/5 | 104.2 |

Compared with the positive control hu24-ADC, all the four anti-PTK7/TROP2 bispecific ADCs 3C4-18E9-ADC, 3C4-6F7-ADC, 3E6-18E9-ADC and 3E6-6F7-ADC exhibited better tumor inhibitory effect at both 5 mg/kg dose level and 10 mg/kg dose level, and higher doses led to better therapeutic effects.

The mouse tumor volume and survival after Day 25 were continued to be monitored. On Day 28, all mice died in group G1 and G2; mice in groups G3-G10 survived better, with tumor disappeared in 2 mice in G7 group, 4 mice in G8 group, 2 mice in G9 group, and 2 mice in G10 group. At the end of the experiment on Day 74, all mice died in groups G1-G4 and G6; 4 mice survived in group G5; 4 mice survived in group G7 with tumor disappeared in 3 mice; 5 mice survived in group G8 with tumor disappeared in 4 mice; 5 mice survived in group G9 with tumor disappeared in all 5 mice. This indicates that the anti-PTK7/TROP2 ADCs exhibit significant and sustained anti-tumor activity in vivo.

### Example 8. Anti-Tumor Activity in Human Lung Cancer PDX Model

The ADCs were tested for the effect in human lung cancer patient-derived xenograft model. The percentage of PTK7 positive cells and TROP2 positive cells in the tumor tissue were 77.91% and 91.17% respectively by immunofluorescence test. B-NDG mice were engrafted in the right flank with the patient-derived tumor fragment (2 mm×2 mm×2 mm). When the tumors in the mice reached a volume of about 150 mm³, the mice were randomly placed into different groups based on the volume of the tumor. The mice were then injected with PBS or ADCs by i.v. administration. Details of the administration scheme are shown in the table below.

TABLE 17

| | | | | | | Total |
|---|---|---|---|---|---|---|
| | No. | | | | | No. of |
| | of | | | | Fre- | admin- |
| Group | mice | ADC | Dosage | Route | quency | istration |
| G1 | 5 | PBS | — | i.v. | QW | 2 |
| G2 | 5 | hu24-ADC | 6 mg/kg | i.v. | QW | 2 |
| G3 | 5 | Sacituzumab | 6 mg/kg | i.v. | QW | 2 |
| | | govitecan | | | | |
| G4 | 5 | 3C4-6F7- | 6 mg/kg | i.v. | QW | 2 |
| | | ADC | | | | |
| G5 | 5 | 3E6-6F7- | 6 mg/kg | i.v. | QW | 2 |
| | | ADC | | | | |
| G6 | 5 | 3C4-18E9- | 6 mg/kg | i.v. | QW | 2 |
| | | ADC | | | | |
| G7 | 5 | 3E6-18E9- | 6 mg/kg | i.v. | QW | 2 |
| | | ADC | | | | |

The table below summarizes the results for this experiment, including the tumor volumes on Day 0, Day 14, and Day 21; TGI; and the statistical differences (P value) of tumor volume between the treatment and control groups on Day 21.

TABLE 18

| | Tumor volume (mm³) | | | TGI | |
|---|---|---|---|---|---|
| Group | Day 0 | Day 14 | Day 21 | (%) | P value |
| G1 | 166 ± 12 | 1414 ± 115 | 2058 ± 143 | NA | NA |
| G2 | 166 ± 20 | 428 ± 69 | 722 ± 141 | 70.6 | 1.61E−04 |
| G3 | 166 ± 18 | 496 ± 137 | 1042 ± 353 | 53.7 | 0.028 |
| G4 | 166 ± 15 | 129 ± 39 | 160 ± 51 | 100.3 | 1.55E−06 |
| G5 | 166 ± 19 | 233 ± 132 | 252 ± 123 | 95.4 | 3.48E−05 |
| G6 | 166 ± 18 | 225 ± 74 | 426 ± 113 | 86.3 | 1.88E−05 |
| G7 | 166 ± 19 | 355 ± 38 | 620 ± 81 | 76.0 | 2.27E−05 |

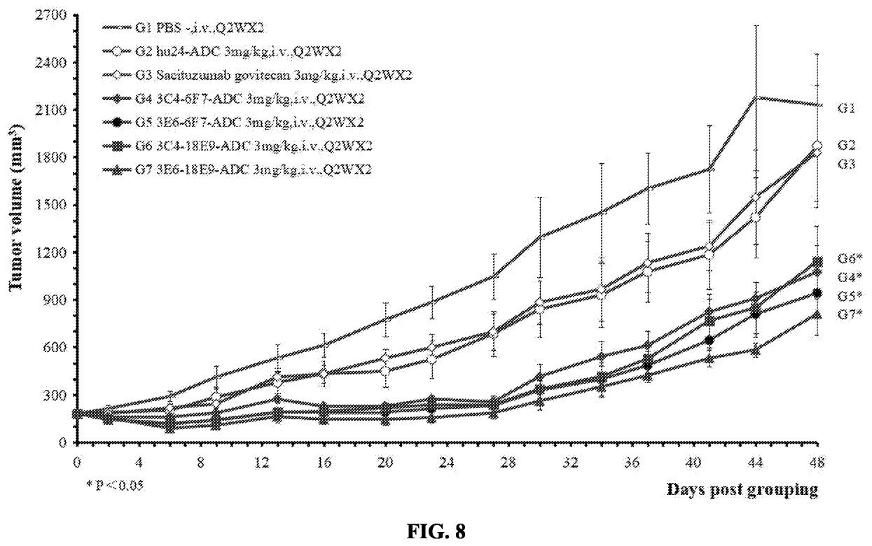

The tumor volume of mice in different groups treated with the ADCs or PBS are shown in FIG. 7. All the four anti-PTK7/TROP2 bispecific ADCs 3C4-18E9-ADC, 3C4-6F7-ADC, 3E6-18E9-ADC and 3E6-6F7-ADC exhibited better tumor inhibitory effect than that of the positive controls hu24-ADC and Sacituzumab govitecan.

### Example 9. Anti-Tumor Activity in Human Pancreatic Cancer PDX Model

The ADCs were tested for the effect in human pancreatic cancer patient-derived xenograft model. The percentage of PTK7 positive cells and TROP2 positive cells in the tumor tissue were 30.07% and 86.74% respectively by immunofluorescence test. B-NDG mice were engrafted in the right flank with the patient-derived tumor fragment (2 mm×2 mm×2 mm). When the tumors in the mice reached a volume of about 200 mm³, the mice were randomly placed into different groups based on the volume of the tumor. The mice were then injected with PBS or ADCs by i.v. administration. Details of the administration scheme are shown in the table below.

TABLE 19

| | | | | | | Total |
|---|---|---|---|---|---|---|
| | No. | | | | | No. of |
| | of | | | | Fre- | admin- |
| Group | mice | ADC | Dosage | Route | quency | istration |
| G1 | 5 | PBS | — | i.v. | Q2W | 2 |
| G2 | 5 | hu24-ADC | 3 mg/kg | i.v. | Q2W | 2 |
| G3 | 5 | Sacituzumab | 3 mg/kg | i.v. | Q2W | 2 |
| | | govitecan | | | | |
| G4 | 5 | 3C4-6F7- | 3 mg/kg | i.v. | Q2W | 2 |
| | | ADC | | | | |
| G5 | 5 | 3E6-6F7- | 3 mg/kg | i.v. | Q2W | 2 |
| | | ADC | | | | |
| G6 | 5 | 3C4-18E9- | 3 mg/kg | i.v. | Q2W | 2 |
| | | ADC | | | | |
| G7 | 5 | 3E6-18E9- | 3 mg/kg | i.v | Q2W | 2 |
| | | ADC | | | | |

The weights of mice in different groups all increased. On the day of group assignment (Day 0), the average weight of each group was in the range of 21.6 g-23.2 g. 48 days post grouping (Day 48), the average body weight of each group was in the range of 22.4 g-23.8 g, and the average body weight change of each group was in the range of 101.3%-105.8%. The results showed that these tested ADCs were well tolerated and were not obviously toxic to the mice.

The table below summarizes the results for this experiment, including the tumor volumes on Day 0, Day 16, D34 and Day 48; TGI; and the statistical differences (P value) of tumor volume between the treatment and control groups on Day 48.

TABLE 20

| | Tumor volume (mm³) | | | | TGI | P value | |
|---|---|---|---|---|---|---|---|
| Group | Day 0 | Day 16 | Day 34 | Day 48 | (%) | Tumor volume | Body weight |
| G1 | 180 ± 18 | 611 ± 73 | 1451 ± 313 | 2130 ± 326 | NA | NA | NA |
| G2 | 179 ± 23 | 432 ± 80 | 929 ± 202 | 1871 ± 386 | 13.3 | 0.636 | 0.775 |
| G3 | 180 ± 22 | 431 ± 56 | 966 ± 198 | 1828 ± 303 | 15.5 | 0.521 | 0.590 |
| G4 | 180 ± 17 | 227 ± 12 | 541 ± 96 | 1076 ± 171 | 54.1 | 0.019 | 0.703 |
| G5 | 180 ± 21 | 186 ± 39 | 397 ± 104 | 942 ± 128 | 60.9 | 0.008 | 0.766 |
| G6 | 180 ± 20 | 195 ± 28 | 415 ± 69 | 1141 ± 224 | 50.7 | 0.036 | 0.992 |
| G7 | 180 ± 24 | 144 ± 28 | 350 ± 65 | 810 ± 132 | 67.7 | 0.005 | 0.508 |

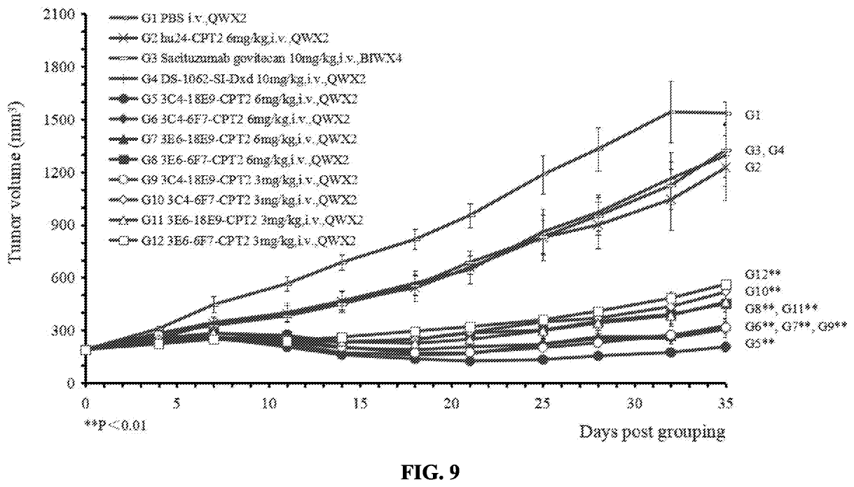

The tumor volume of mice in different groups treated with the ADCs or PBS are shown in FIG. 8. All the four anti-PTK7/TROP2 bispecific ADCs 3C4-18E9-ADC, 3C4-6F7-ADC, 3E6-18E9-ADC and 3E6-6F7-ADC exhibited better tumor inhibitory effect than that of the positive controls hu24-ADC and Sacituzumab govitecan.

### Example 10. Antibody Drug Conjugates

The purified antibodies were also coupled with CPT-1, CPT-2, CPT-3, or CPT-4, through CPT-L linker. For the names of antibody-drug conjugates, CPTx is added directly after the antibody name. For example, if 3C4 is coupled to CPT-1, it is named as 3C4-CPT1. For example, if 3C4 is coupled to CPT-2, it is named as 3C4-CPT2. MS (Mass Spectrometry) was used to detect the coupling of antibodies with drug molecules. The MS detection results showed that the drug-to-antibody ratio (DAR) of the ADCs was about 8.

For reference purpose, DS-1062-SI was coupled to Dxd through GGFG linker to form DS-1062-SI-Dxd, and the DAR was about 4.

### Example 11. Anti-Tumor Activity in Human Colorectal Cancer PDX Model

The ADCs were tested for the effect in human colorectal cancer patient-derived xenograft model. B-NDG mice were engrafted in the right flank with the patient-derived tumor fragment (2 mm×2 mm×2 mm). When the tumors in the mice reached a volume of about 200 mm³, the mice were randomly placed into different groups based on the volume of the tumor. The mice were then injected with PBS or ADCs by i.v. administration. Details of the administration scheme are shown in the table below.

TABLE 21

| | | | | | | Total |
|---|---|---|---|---|---|---|
| | No. | | | | | No. of |
| | of | | | | Fre- | admin- |
| Group | mice | ADC | Dosage | Route | quency | istration |
| G1 | 5 | PBS | — | i.v. | QW | 2 |
| G2 | 5 | hu24-CPT2 | 6 mg/kg | i.v. | QW | 2 |
| G3 | 5 | Sacituzumab | 10 mg/kg | i.v. | BIW | 4 |
| | | govitecan | | | | |
| G4 | 5 | DS-1062- | 10 mg/kg | i.v. | QW | 2 |
| | | SI-Dxd | | | | |
| G5 | 5 | 3C4-18E9- | 6 mg/kg | i.v. | QW | 2 |
| | | CPT2 | | | | |
| G6 | 5 | 3C4-6F7- | 6 mg/kg | i.v. | QW | 2 |
| | | CPT2 | | | | |
| G7 | 5 | 3E6-18E9- | 6 mg/kg | i.v. | QW | 2 |
| | | CPT2 | | | | |
| G8 | 5 | 3E6-6F7- | 6 mg/kg | i.v. | QW | 2 |
| | | CPT2 | | | | |
| G9 | 5 | 3C4-18E9- | 3 mg/kg | i.v. | QW | 2 |
| | | CPT2 | | | | |
| G10 | 5 | 3C4-6F7- | 3 mg/kg | i.v. | QW | 2 |
| | | CPT2 | | | | |
| G11 | 5 | 3E6-18E9- | 3 mg/kg | i.v. | QW | 2 |
| | | CPT2 | | | | |
| G12 | 5 | 3E6-6F7- | 3 mg/kg | i.v. | QW | 2 |
| | | CPT2 | | | | |

As shown in FIG. 9, the tumor volumes in all treatment groups (G2-G12) were smaller than those in the control group (G1). The results also showed that 3C4-18E9-CPT2 (G5, G9), 3C4-6F7-CPT2 (G6, G10), 3E6-18E9-CPT2 (G7, G11) and 3E6-6F7-CPT2 (G8, G12) exhibited better anti-tumor effects than that of the positive controls (G2-G4) both at a dose level of 3 mg/kg or 6 mg/kg, with a TGI % (e.g., on Day 35) of 98.8% (G5), 91.4% (G6), 89.7% (G7), 80.6% (G8), 90.6% (G9), 75.6% (G10), 79.9% (G11) and 72.4% (G12), respectively.

The mouse tumor volume and survival after Day 35 were continued to be monitored. On Day 60 post grouping, all mice in G1 group died; 4 mice died in G2 and G4 groups; 3 mice died in G3 group; only one mouse died in G6, G9 and G10; all mice in G5, G7, G11 and G12 groups survived, indicating that 3C4-18E9-CPT2 (G5, G9), 3C4-6F7-CPT2 (G6, G10), 3E6-18E9-CPT2 (G7, G11) and 3E6-6F7-CPT2 (G8, G12) have excellent tumor inhibitory effects and good safety.

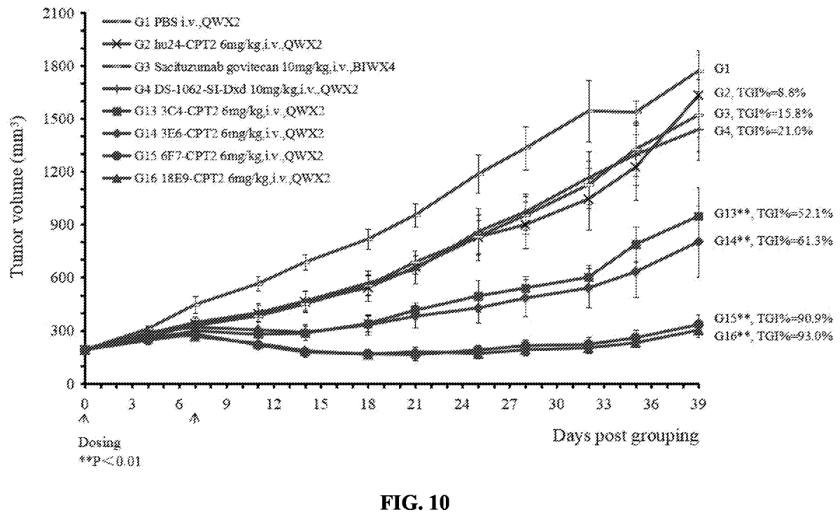

The anti-tumor activities of 3C4-CPT2, 3E6-CPT2, 6F7-CPT2 and 18E9-CPT2 were also tested in the colorectal cancer PDX models. As shown in FIG. 10, 3C4-CPT2 (G13) and 3E6-CPT2 (G14) induced significant tumor growth inhibition at 6 mg/kg with a higher TGI % (e.g., on Day 39) than that of the positive control hu24-CPT2 (G2); 6F7-CPT2 (G15) and 18E9-CPT2 (G16) at 6 mg/kg exhibited better anti-tumor activities than that of the positive controls Sacituzumab govitecan (G3) and DS-1062-SI-Dxd (G4) at a dose level of 10 mg/kg.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to PTK7 (protein tyrosine kinase 7) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 4-6, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 1-3, respectively;
(5) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 1-3, respectively;
(6) the selected VH CDRs 1, 2, 3 amino acid sequences are set forth in SEQ ID NOs: 19-21, respectively, and the selected VL CDRs 1, 2, 3 amino acid sequences are set forth in SEQ ID NOs: 1-3, respectively;
(7) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 1-3, respectively;
(8) the selected VH CDRs 1, 2, 3 amino acid sequences are set forth in SEQ ID NOs: 25-27, respectively, and the selected VL CDRs 1, 2, 3 amino acid sequences are set forth in SEQ ID NOs: 1-3,
(9) the selected VH CDRs 1, 2, 3 amino acid sequences are set forth in SEQ ID NOs: 28-30, respectively, and the selected VL CDRs 1, 2, 3 amino acid sequences are set forth in SEQ ID NOs: 1-3,
(10) the selected VH CDRs 1, 2, 3 amino acid sequences are set forth in SEQ ID NOs: 31-33, respectively, and the selected VL CDRs 1, 2, 3 amino acid sequences are set forth in SEQ ID NOs: 1-3, respectively;
(11) the selected VH CDRs 1, 2, 3 amino acid sequences are set forth in SEQ ID NOs: 34-36, respectively, and the selected VL CDRs 1, 2, 3 amino acid sequences are set forth in SEQ ID NOs: 1-3, respectively;
(12) the selected VH CDRs 1, 2, 3 amino acid sequences are set forth in SEQ ID NOs: 37-39, respectively, and the selected VL CDRs 1, 2, 3 amino acid sequences are set forth in SEQ ID NOs: 1-3, respectively;
(13) the selected VH CDRs 1, 2, 3 amino acid sequences are set forth in SEQ ID NOs: 40-42, respectively, and the selected VL CDRs 1, 2, 3 amino acid sequences are set forth in SEQ ID NOs: 1-3, respectively;
(14) the selected VH CDRs 1, 2, 3 amino acid sequences are set forth in SEQ ID NOs: 43-45, respectively, and the selected VL CDRs 1, 2, 3 amino acid sequences are set forth in SEQ ID NOs: 1-3, respectively;
(15) the selected VH CDRs 1, 2, 3 amino acid sequences are set forth in SEQ ID NOs: 86-88, respectively, and the selected VL CDRs 1, 2, 3 amino acid sequences are set forth in SEQ ID NOs: 1-3, respectively; and
(16) the selected VH CDRs 1, 2, 3 amino acid sequences are set forth in SEQ ID NOs: 89-91, respectively, and the selected VL CDRs 1, 2, 3 amino acid sequences are set forth in SEQ ID NOs: 1-3, respectively.

2. The antibody or antigen-binding fragment thereof of claim 1, wherein the VH comprises CDRs 1, 2, 3 with the amino acid sequences set forth in SEQ ID NOs: 4-6, respectively, and the VL comprises CDRs 1, 2, 3 with the amino acid sequences set forth in SEQ ID NOs: 1-3, respectively, according to Kabat definition.

3. The antibody or antigen-binding fragment thereof of claim 1, wherein the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 1-3, respectively, according to Kabat definition.

4. The antibody or antigen-binding fragment thereof of claim 1, wherein the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 1-3, respectively, according to Kabat definition.

5. The antibody or antigen-binding fragment thereof of claim 1, wherein the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 1-3, respectively, according to Kabat definition.

6. The antibody or antigen-binding fragment thereof of claim 1, wherein the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 1-3, respectively, according to Kabat definition.

7. The antibody or antigen-binding fragment thereof of claim 1, wherein the VH comprises CDRs 1, 2, 3 with the amino acid sequences set forth in SEQ ID NOs: 19-21, respectively, and the VL comprises CDRs 1, 2, 3 with the amino acid sequences set forth in SEQ ID NOs: 1-3, respectively, according to Kabat definition.

8. The antibody or antigen-binding fragment thereof of claim 1, wherein the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 1-3, respectively, according to Kabat definition.

9. The antibody or antigen-binding fragment thereof of claim 1, wherein the VH comprises CDRs 1, 2, 3 with the amino acid sequences set forth in SEQ ID NOs: 86-88, respectively, and the VL comprises CDRs 1, 2, 3 with the amino acid sequences set forth in SEQ ID NOs: 1-3, respectively, according to Kabat definition.

10. The antibody or antigen-binding fragment thereof of claim 1, wherein the VH comprises CDRs 1, 2, 3 with the amino acid sequences set forth in SEQ ID NOs: 25-27, respectively, and the VL comprises CDRs 1, 2, 3 with the amino acid sequences set forth in SEQ ID NOs: 1-3, respectively, according to Chothia definition.

11. The antibody or antigen-binding fragment thereof of claim 1, wherein the VH comprises CDRs 1, 2, 3 with the amino acid sequences set forth in SEQ ID NOs: 28-30, respectively, and the VL comprises CDRs 1, 2, 3 with the amino acid sequences set forth in SEQ ID NOs: 1-3, respectively, according to Chothia definition.

12. The antibody or antigen-binding fragment thereof of claim 1, wherein the VH comprises CDRs 1, 2, 3 with the amino acid sequences set forth in SEQ ID NOs: 31-33, respectively, and the VL comprises CDRs 1, 2, 3 with the amino acid sequences set forth in SEQ ID NOs: 1-3, respectively, according to Chothia definition.

13. The antibody or antigen-binding fragment thereof of claim 1, wherein the VH comprises CDRs 1, 2, 3 with the amino acid sequences set forth in SEQ ID NOs: 34-36, respectively, and the VL comprises CDRs 1, 2, 3 with the amino acid sequences set forth in SEQ ID NOs: 1-3, respectively, according to Chothia definition.

14. The antibody or antigen-binding fragment thereof of claim 1, wherein the VH comprises CDRs 1, 2, 3 with the amino acid sequences set forth in SEQ ID NOs: 37-39, respectively, and the VL comprises CDRs 1, 2, 3 with the amino acid sequences set forth in SEQ ID NOs: 1-3, respectively, according to Chothia definition.

15. The antibody or antigen-binding fragment thereof of claim 1, wherein the VH comprises CDRs 1, 2, 3 with the amino acid sequences set forth in SEQ ID NOs: 40-42, respectively, and the VL comprises CDRs 1, 2, 3 with the amino acid sequences set forth in SEQ ID NOs: 1-3, respectively, according to Chothia definition.

16. The antibody or antigen-binding fragment thereof of claim 1, wherein the VH comprises CDRs 1, 2, 3 with the amino acid sequences set forth in SEQ ID NOs: 43-45, respectively, and the VL comprises CDRs 1, 2, 3 with the amino acid sequences set forth in SEQ ID NOs: 1-3, respectively, according to Chothia definition.

17. The antibody or antigen-binding fragment thereof of claim 1, wherein the VH comprises CDRs 1, 2, 3 with the amino acid sequences set forth in SEQ ID NOs: 89-91, respectively, and the VL comprises CDRs 1, 2, 3 with the amino acid sequences set forth in SEQ ID NOs: 1-3, respectively, according to Kabat definition.

18. The antibody or antigen-binding fragment thereof of any one of claims 1-17, wherein the antibody or antigen-binding fragment thereof specifically binds to human, monkey, or dog PTK7.

19. The antibody or antigen-binding fragment thereof of any one of claims 1-18, wherein the antibody or antigen-binding fragment thereof is a human antibody or antigen-binding fragment thereof, a single-chain variable fragment (scFv), a one-armed antibody, and/or a multi-specific antibody (e.g., a bispecific antibody).

20. The antibody or antigen-binding fragment thereof of any one of claims 1-19, wherein the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

21. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 4-6, respectively, and wherein the VH, when paired with a light chain variable region (VL) comprising the amino acid sequence set forth in SEQ ID NO: 53 binds to PTK7;
(2) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 25-27, respectively, and wherein the VH, when paired with a light chain variable region (VL) comprising the amino acid sequence set forth in SEQ ID NO: 53 binds to PTK7;
(3) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 46 binds to PTK7;
(4) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7-9, respectively, and wherein the VH, when paired with a light chain variable region (VL) comprising the amino acid sequence set forth in SEQ ID NO: 53 binds to PTK7;
(5) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 28-30, respectively, and wherein the VH, when paired with a light chain variable region (VL) comprising the amino acid sequence set forth in SEQ ID NO: 53 binds to PTK7;
(6) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 47 binds to PTK7;
(7) an immunoglobulin heavy chain or a fragment thereof comprising a VH comprising complementarity determining regions (CDRs) 1, 2, and 3 comprising the amino acid sequences set forth in SEQ ID NOs: 10-12, respectively, and wherein the VH, when paired with a light chain variable region (VL) comprising the amino acid sequence set forth in SEQ ID NO: 53 binds to PTK7;
(8) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 31-33, respectively, and wherein the VH, when paired with a light chain variable region (VL) comprising the amino acid sequence set forth in SEQ ID NO: 53 binds to PTK7;
(9) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 48 binds to PTK7;
(10) an immunoglobulin heavy chain or a fragment thereof comprising a VH comprising complementarity determining regions (CDRs) 1, 2, and 3 comprising the amino acid sequences set forth in SEQ ID NOs: 13-15, respectively, and wherein the VH, when paired with a light chain variable region (VL) comprising the amino acid sequence set forth in SEQ ID NO: 53 binds to PTK7;
(11) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 34-36, respectively, and wherein the VH, when paired with a light chain variable region (VL) comprising the amino acid sequence set forth in SEQ ID NO: 53 binds to PTK7;
(12) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 49 binds to PTK7;
(13) an immunoglobulin heavy chain or a fragment thereof comprising a VH comprising complementarity determining regions (CDRs) 1, 2, and 3 comprising the amino acid sequences set forth in SEQ ID NOs: 16-18, respectively, and wherein the VH, when paired with a light chain variable region (VL) comprising the amino acid sequence set forth in SEQ ID NO: 53 binds to PTK7;
(14) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 37-39, respectively, and wherein the VH, when paired with a light chain variable region (VL) comprising the amino acid sequence set forth in SEQ ID NO: 53 binds to PTK7;
(15) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 50 binds to PTK7;
(16) an immunoglobulin heavy chain or a fragment thereof comprising a VH comprising complementarity determining regions (CDRs) 1, 2, and 3 comprising the amino acid sequences set forth in SEQ ID NOs: 19-21, respectively, and wherein the VH, when paired with a light chain variable region (VL) comprising the amino acid sequence set forth in SEQ ID NO: 53 binds to PTK7;
(17) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 40-42, respectively, and wherein the VH, when paired with a light chain variable region (VL) comprising the amino acid sequence set forth in SEQ ID NO: 53 binds to PTK7;
(18) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 51 binds to PTK7;
(19) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 22-24, respectively, and wherein the VH, when paired with a light chain variable region (VL) comprising the amino acid sequence set forth in SEQ ID NO: 53 binds to PTK7;
(20) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 43-45, respectively, and wherein the VH, when paired with a light chain variable region (VL) comprising the amino acid sequence set forth in SEQ ID NO: 53 binds to PTK7; and
(21) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 52 binds to PTK7.
(22) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 86-88, respectively, and wherein the VH, when paired with a light chain variable region (VL) comprising the amino acid sequence set forth in SEQ ID NO: 53 binds to PTK7;
(23) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 89-91, respectively, and wherein the VH, when paired with a light chain variable region (VL) comprising the amino acid sequence set forth in SEQ ID NO: 53 binds to PTK7; and
(24) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 92 binds to PTK7.

22. The nucleic acid of claim 21, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1-3, respectively.

23. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 4-6, respectively.

24. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7-9, respectively.

25. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 10-12, respectively.

26. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13-15, respectively.

27. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 16-18, respectively.

28. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 19-21, respectively.

29. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 22-24, respectively.

30. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 25-27, respectively.

31. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 28-30, respectively.

32. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 31-33, respectively.

33. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 34-36, respectively.

34. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 37-39, respectively.

35. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 40-42, respectively.

36. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 43-45, respectively.

37. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 86-88, respectively.

38. The nucleic acid of claim 21, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 89-91, respectively.

39. The nucleic acid of any one of claims 21-38, wherein the VH when paired with a VL specifically binds to human, monkey, or dog PTK7, or the VL when paired with a VH specifically binds to human, monkey, or dog PTK7.

40. The nucleic acid of any one of claims 21-39, wherein the immunoglobulin heavy chain or the fragment thereof is a human immunoglobulin heavy chain or a fragment thereof (e.g., a human IgG1 heavy chain or a fragment thereof, a human IgG2 antibody or antigen-binding fragment thereof, or a human IgG4 heavy chain or a fragment thereof), and the immunoglobulin light chain or the fragment thereof is a human immunoglobulin light chain or a fragment thereof.

41. The nucleic acid of any one of claims 21-40, wherein the nucleic acid encodes a single-chain variable fragment (scFv), a one-armed antibody, a multi-specific antibody (e.g., a bispecific antibody), or a chimeric antigen receptor (CAR).

42. The nucleic acid of any one of claims 21-41, wherein the nucleic acid is cDNA.

43. A vector comprising one or more of the nucleic acids of any one of claims 21-42.

44. A vector comprising two of the nucleic acids of any one of claims 21-42, wherein the vector encodes the VL region and the VH region that together bind to PTK7.

45. A pair of vectors, wherein each vector comprises one of the nucleic acids of any one of claims 21-42, wherein together the pair of vectors encodes the VL region and the VH region that together bind to PTK7.

46. A cell comprising the vector of claim 43 or 44, or the pair of vectors of claim 45.

47. The cell of claim 46, wherein the cell is a CHO cell.

48. A cell comprising one or more of the nucleic acids of any one of claims 21-42.

49. A cell comprising two of the nucleic acids of any one of claims 21-42.

50. The cell of claim 49, wherein the two nucleic acids together encode the VL region and the VH region that together bind to PTK7.

51. A method of producing an antibody or an antigen-binding fragment thereof, the method comprising
(a) culturing the cell of any one of claims 46-50 under conditions sufficient for the cell to produce the antibody or the antigen-binding fragment; and
(b) collecting the antibody or the antigen-binding fragment produced by the cell.

52. An antibody or antigen-binding fragment thereof that binds to PTK7 comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 46, and the selected VL sequence is SEQ ID NO: 53;
(2) the selected VH sequence is SEQ ID NO: 47, and the selected VL sequence is SEQ ID NO: 53;
(3) the selected VH sequence is SEQ ID NO: 48, and the selected VL sequence is SEQ ID NO: 53;
(4) the selected VH sequence is SEQ ID NO: 49, and the selected VL sequence is SEQ ID NO: 53;
(5) the selected VH sequence is SEQ ID NO: 50, and the selected VL sequence is SEQ ID NO: 53;
(6) the selected VH sequence is SEQ ID NO: 51, and the selected VL sequence is SEQ ID NO: 53;
(7) the selected VH sequence is SEQ ID NO: 52, and the selected VL sequence is SEQ ID NO: 53; and
(8) the selected VH sequence is SEQ ID NO: 92, and the selected VL sequence is SEQ ID NO: 53.

53. The antibody or antigen-binding fragment thereof of claim 52, wherein the VH comprises the sequence of SEQ ID NO: 46 and the VL comprises the sequence of SEQ ID NO: 53.

54. The antibody or antigen-binding fragment thereof of claim 52, wherein the VH comprises the sequence of SEQ ID NO: 47 and the VL comprises the sequence of SEQ ID NO: 53.

55. The antibody or antigen-binding fragment thereof of claim 52, wherein the VH comprises the sequence of SEQ ID NO: 48 and the VL comprises the sequence of SEQ ID NO: 53.

56. The antibody or antigen-binding fragment thereof of claim 52, wherein the VH comprises the sequence of SEQ ID NO: 49 and the VL comprises the sequence of SEQ ID NO: 53.

57. The antibody or antigen-binding fragment thereof of claim 52, wherein the VH comprises the sequence of SEQ ID NO: 50 and the VL comprises the sequence of SEQ ID NO: 53.

58. The antibody or antigen-binding fragment thereof of claim 52, wherein the VH comprises the sequence of SEQ ID NO: 51 and the VL comprises the sequence of SEQ ID NO: 53.

59. The antibody or antigen-binding fragment thereof of claim 52, wherein the VH comprises the sequence of SEQ ID NO: 52 and the VL comprises the sequence of SEQ ID NO: 53.

60. The antibody or antigen-binding fragment thereof of claim 52, wherein the VH comprises the sequence of SEQ ID NO: 92 and the VL comprises the sequence of SEQ ID NO: 53.

61. An antibody or antigen-binding fragment thereof that binds to PTK7 comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 46, and the selected VL sequence is SEQ ID NO: 53;
(2) the selected VH sequence is SEQ ID NO: 47, and the selected VL sequence is SEQ ID NO: 53;
(3) the selected VH sequence is SEQ ID NO: 48, and the selected VL sequence is SEQ ID NO: 53;
(4) the selected VH sequence is SEQ ID NO: 49, and the selected VL sequence is SEQ ID NO: 53;
(5) the selected VH sequence is SEQ ID NO: 50, and the selected VL sequence is SEQ ID NO: 53;
(6) the selected VH sequence is SEQ ID NO: 51, and the selected VL sequence is SEQ ID NO: 53;
(7) the selected VH sequence is SEQ ID NO: 52, and the selected VL sequence is SEQ ID NO: 53; and
(8) the selected VH sequence is SEQ ID NO: 92, and the selected VL sequence is SEQ ID NO: 53.

62. The antibody or antigen-binding fragment thereof of any one of claims 52-61, wherein the antibody or antigen-binding fragment thereof specifically binds to human, monkey, or dog PTK7.

63. The antibody or antigen-binding fragment thereof of any one of claims 52-62, wherein the antibody or antigen-binding fragment thereof is a human antibody or antigen-binding fragment thereof, a single-chain variable fragment (scFv), a one-armed antibody, and/or a multi-specific antibody (e.g., a bispecific antibody).

64. The antibody or antigen-binding fragment thereof of any one of claims 52-63, wherein the antibody or antigen-binding fragment is a human IgG1 antibody or antigen-binding fragment thereof, a human IgG2 antibody or antigen-binding fragment thereof, or a human IgG4 antibody or antigen-binding fragment thereof.

65. An antibody or antigen-binding fragment thereof that binds to TROP2 (trophoblast cell surface antigen 2) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 57-59, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 60-62, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 66-68, respectively, and the selected VL CDRs 1, 2, 3 amino acid sequences are set forth in SEQ ID NOs: 1-3, respectively; and
(4) the selected VH CDRs 1, 2, 3 amino acid sequences are set forth in SEQ ID NOs: 69-71, respectively, and the selected VL CDRs 1, 2, 3 amino acid sequences are set forth in SEQ ID NOs: 1-3, respectively.

66. The antibody or antigen-binding fragment thereof of claim 65, wherein the VH comprises CDRs 1, 2, 3 with the amino acid sequences set forth in SEQ ID NOs: 57-59, respectively, and the VL comprises CDRs 1, 2, 3 with the amino acid sequences set forth in SEQ ID NOs: 1-3, respectively, according to Kabat definition.

67. The antibody or antigen-binding fragment thereof of claim 65, wherein the VH comprises CDRs 1, 2, 3 with the amino acid sequences set forth in SEQ ID NOs: 60-62, respectively, and the VL comprises CDRs 1, 2, 3 with the amino acid sequences set forth in SEQ ID NOs: 1-3, respectively, according to Kabat definition.

68. The antibody or antigen-binding fragment thereof of claim 65, wherein the VH comprises CDRs 1, 2, 3 with the amino acid sequences set forth in SEQ ID NOs: 66-68, respectively, and the VL comprises CDRs 1, 2, 3 with the amino acid sequences set forth in SEQ ID NOs: 1-3, respectively, according to Chothia definition.

69. The antibody or antigen-binding fragment thereof of claim 65, wherein the VH comprises CDRs 1, 2, 3 with the amino acid sequences set forth in SEQ ID NOs: 69-71, respectively, and the VL comprises CDRs 1, 2, 3 with the amino acid sequences set forth in SEQ ID NOs: 1-3, respectively, according to Chothia definition.

70. The antibody or antigen-binding fragment thereof of any one of claims 65-69, wherein the antibody or antigen-binding fragment thereof specifically binds to human, monkey, or dog TROP2.

71. The antibody or antigen-binding fragment thereof of any one of claims 65-70, wherein the antibody or antigen-binding fragment thereof is a human antibody or antigen-binding fragment thereof, a single-chain variable fragment (scFv), a one-armed antibody, and/or a multi-specific antibody (e.g., a bispecific antibody).

72. The antibody or antigen-binding fragment thereof of any one of claims 65-71, wherein the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

73. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 57-59, respectively, and wherein the VH, when paired with a light chain variable region (VL) comprising the amino acid sequence set forth in SEQ ID NO: 53 binds to TROP2;
(2) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 66-68, respectively, and wherein the VH, when paired with a light chain variable region (VL) comprising the amino acid sequence set forth in SEQ ID NO: 53 binds to TROP2;
(3) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 73 binds to TROP2;
(4) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 60-62, respectively, and wherein the VH, when paired with a light chain variable region (VL) comprising the amino acid sequence set forth in SEQ ID NO: 53 binds to TROP2;
(5) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 69-71, respectively, and wherein the VH, when paired with a light chain variable region (VL) comprising the amino acid sequence set forth in SEQ ID NO: 53 binds to TROP2; and
(6) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 74 binds to TROP2.

74. The nucleic acid of claim 73, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1-3, respectively.

75. The nucleic acid of claim 73, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 57-59, respectively.

76. The nucleic acid of claim 73, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 60-62, respectively.

77. The nucleic acid of claim 73, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 66-68, respectively.

78. The nucleic acid of claim 73, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 69-71, respectively.

79. The nucleic acid of any one of claims 73-78, wherein the VH when paired with a VL specifically binds to human, monkey, or dog TROP2, or the VL when paired with a VH specifically binds to human, monkey, or dog TROP2.

80. The nucleic acid of any one of claims 73-79, wherein the immunoglobulin heavy chain or the fragment thereof is a human immunoglobulin heavy chain or a fragment thereof (e.g., a human IgG1 heavy chain or a fragment thereof, a human IgG2 antibody or antigen-binding fragment thereof, or a human IgG4 heavy chain or a fragment thereof), and the immunoglobulin light chain or the fragment thereof is a human immunoglobulin light chain or a fragment thereof.

81. The nucleic acid of any one of claims 73-80, wherein the nucleic acid encodes a single-chain variable fragment (scFv), a one-armed antibody, a multi-specific antibody (e.g., a bispecific antibody), or a chimeric antigen receptor (CAR).

82. The nucleic acid of any one of claims 73-81, wherein the nucleic acid is cDNA.

83. A vector comprising one or more of the nucleic acids of any one of claims 73-82.

84. A vector comprising two of the nucleic acids of any one of claims 73-82, wherein the vector encodes the VL region and the VH region that together bind to TROP2.

85. A pair of vectors, wherein each vector comprises one of the nucleic acids of any one of claims 73-82, wherein together the pair of vectors encodes the VL region and the VH region that together bind to TROP2.

86. A cell comprising the vector of claim 83 or 84, or the pair of vectors of claim 85.

87. The cell of claim 86, wherein the cell is a CHO cell.

88. A cell comprising one or more of the nucleic acids of any one of claims 73-82.

89. A cell comprising two of the nucleic acids of any one of claims 73-82.

90. The cell of claim 89, wherein the two nucleic acids together encode the VL region and the VH region that together bind to TROP2.

91. A method of producing an antibody or an antigen-binding fragment thereof, the method comprising
(a) culturing the cell of any one of claims 86-90 under conditions sufficient for the cell to produce the antibody or the antigen-binding fragment; and
(b) collecting the antibody or the antigen-binding fragment produced by the cell.

92. An antibody or antigen-binding fragment thereof that binds to TROP2 comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 73, and the selected VL sequence is SEQ ID NO: 53; and
(2) the selected VH sequence is SEQ ID NO: 74, and the selected VL sequence is SEQ ID NO: 53.

93. The antibody or antigen-binding fragment thereof of claim 92, wherein the VH comprises the sequence of SEQ ID NO: 73 and the VL comprises the sequence of SEQ ID NO: 53.

94. The antibody or antigen-binding fragment thereof of claim 92, wherein the VH comprises the sequence of SEQ ID NO: 74 and the VL comprises the sequence of SEQ ID NO: 53.

95. An antibody or antigen-binding fragment thereof that binds to TROP2 comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 73, and the selected VL sequence is SEQ ID NO: 53; and
(2) the selected VH sequence is SEQ ID NO: 74, and the selected VL sequence is SEQ ID NO: 53.

96. The antibody or antigen-binding fragment thereof of any one of claims 92-95, wherein the antibody or antigen-binding fragment thereof specifically binds to human, monkey, or dog TROP2.

97. The antibody or antigen-binding fragment thereof of any one of claims 92-96, wherein the antibody or antigen-binding fragment thereof is a human antibody or antigen-binding fragment thereof, a single-chain variable fragment (scFv), a one-armed antibody, and/or a multi-specific antibody (e.g., a bispecific antibody).

98. The antibody or antigen-binding fragment thereof of any one of claims 92-97, wherein the antibody or antigen-binding fragment is a human IgG1 antibody or antigen-binding fragment thereof, a human IgG2 antibody or antigen-binding fragment thereof, or a human IgG4 antibody or antigen-binding fragment thereof.

99. An anti-PTK7/TROP2 antibody or antigen-binding fragment thereof, comprising: a first antigen-binding domain that specifically binds to an epitope of PTK7; and a second antigen-binding domain that specifically binds to an epitope of TROP2.

100. The anti-PTK7/TROP2 antibody or antigen-binding fragment thereof of claim 99, wherein the first antigen-binding domain comprises a first heavy chain variable region (VH1) and a first light chain variable region (VL1); and the second antigen-binding domain comprises a second heavy chain variable region (VH2) and a second light chain variable region (VL2).

101. The anti-PTK7/TROP2 antibody or antigen-binding fragment thereof of claim 100, wherein the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOS: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOS: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOS: 1-3, respectively;
(8) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively;
(10) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively;
(11) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively;
(12) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively;
(13) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively;
(14) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively;
(15) the selected VH1 CDRs 1, 2, 3 amino acid sequences are set forth in SEQ ID NOs: 86-88, respectively, and the selected VL1 CDRs 1, 2, 3 amino acid sequences are set forth in SEQ ID NOs: 1-3, respectively; and
(16) the selected VH1 CDRs 1, 2, 3 amino acid sequences are set forth in SEQ ID NOs: 89-91, respectively, and the selected VL1 CDRs 1, 2, 3 amino acid sequences are set forth in SEQ ID NOs: 1-3, respectively.

102. The anti-PTK7/TROP2 antibody or antigen-binding fragment thereof of claim 100 or claim 101, wherein the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOS: 1-3, respectively.

103. The anti-PTK7/TROP2 antibody or antigen-binding fragment thereof of claim 100 or claim 101, wherein the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively.

104. The anti-PTK7/TROP2 antibody or antigen-binding fragment thereof of claim 100 or claim 101, wherein the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively.

105. The anti-PTK7/TROP2 antibody or antigen-binding fragment thereof of claim 100 or claim 101, wherein the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively.

106. The anti-PTK7/TROP2 antibody or antigen-binding fragment thereof of claim 100 or claim 101, wherein the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively.

107. The anti-PTK7/TROP2 antibody or antigen-binding fragment thereof of claim 100 or claim 101, wherein the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively.

108. The anti-PTK7/TROP2 antibody or antigen-binding fragment thereof of claim 100 or claim 101, wherein the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOS: 1-3, respectively.

109. The anti-PTK7/TROP2 antibody or antigen-binding fragment thereof of claim 100 or claim 101, wherein the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOS: 1-3, respectively.

110. The anti-PTK7/TROP2 antibody or antigen-binding fragment thereof of claim 100 or claim 101, wherein the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOS: 1-3, respectively.

111. The anti-PTK7/TROP2 antibody or antigen-binding fragment thereof of claim 100 or claim 101, wherein the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOS: 1-3, respectively.

112. The anti-PTK7/TROP2 antibody or antigen-binding fragment thereof of claim 100 or claim 101, wherein the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively.

113. The anti-PTK7/TROP2 antibody or antigen-binding fragment thereof of claim 100 or claim 101, wherein the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively.

114. The anti-PTK7/TROP2 antibody or antigen-binding fragment thereof of claim 100 or claim 101, wherein the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively.

115. The anti-PTK7/TROP2 antibody or antigen-binding fragment thereof of claim 100 or claim 101, wherein the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively.

116. The anti-PTK7/TROP2 antibody or antigen-binding fragment thereof of claim 100 or claim 101, wherein the selected VH1 CDRs 1, 2, 3 amino acid sequences are set forth in SEQ ID NOs: 86-88, respectively, and the selected VL1 CDRs 1, 2, 3 amino acid sequences are set forth in SEQ ID NOs: 1-3, respectively.

117. The anti-PTK7/TROP2 antibody or antigen-binding fragment thereof of claim 100 or claim 101, wherein the selected VH1 CDRs 1, 2, 3 amino acid sequences are set forth in SEQ ID NOs: 89-91, respectively, and the selected VL1 CDRs 1, 2, 3 amino acid sequences are set forth in SEQ ID NOs: 1-3, respectively.

118. The anti-PTK7/TROP2 antibody or antigen-binding fragment thereof of any one of claims 100-117, wherein
the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(4) the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOS: 1-3, respectively;
(5) the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively; and
(6) the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOS: 1-3, respectively.

119. The anti-PTK7/TROP2 antibody or antigen-binding fragment thereof of any one of claims 100-118, wherein the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

120. The anti-PTK7/TROP2 antibody or antigen-binding fragment thereof of any one of claims 100-118, wherein the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOS: 1-3, respectively.

121. The anti-PTK7/TROP2 antibody or antigen-binding fragment thereof of any one of claims 100-118, wherein the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively.

122. The anti-PTK7/TROP2 antibody or antigen-binding fragment thereof of any one of claims 100-118, wherein the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively.

123. The anti-PTK7/TROP2 antibody or antigen-binding fragment thereof of any one of claims 100-118, wherein the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively.

124. The anti-PTK7/TROP2 antibody or antigen-binding fragment thereof of any one of claims 100-118, wherein the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

125. The anti-PTK7/TROP2 antibody or antigen-binding fragment thereof of any one of claims 100-124, wherein
(1) The selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(2) The selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(3) The selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(4) The selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(5) The selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(6) The selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(7) The selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(8) The selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOS: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(9) The selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(10) The selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(11) The selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(12) The selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(13) The selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(14) The selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively;
(15) The selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(16) The selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(17) The selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(18) The selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(19) The selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(20) The selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(21) The selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 1-3, respectively;
(22) The selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(23) The selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(24) The selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(25) The selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(26) The selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(27) The selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(28) The selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(29) The selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively;
(30) The selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively;
(31) The selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively;
(32) The selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively;
(33) The selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively;
(34) The selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively;
(35) The selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively;
(36) The selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(37) The selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(38) The selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(39) The selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(40) The selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(41) The selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(42) The selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(43) The selected VH1 CDRs 1, 2, 3 amino acid sequences are set forth in SEQ ID NOs: 86-88, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(44) The selected VH1 CDRs 1, 2, 3 amino acid sequences are set forth in SEQ ID NOs: 89-91, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 1-3, respectively;
(45) The selected VH1 CDRs 1, 2, 3 amino acid sequences are set forth in SEQ ID NOs: 86-88, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 1-3, respectively; and
(46) The selected VH1 CDRs 1, 2, 3 amino acid sequences are set forth in SEQ ID NOs: 89-91, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 1-3, respectively.

126. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 46, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

127. The anti-PTK7/TROP2 antibody or antigen-binding fragment thereof of any one of claim 100-125, wherein the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

128. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

129. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

130. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

131. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

132. The anti-PTK7/TROP2 antibody or antigen-binding fragment thereof of any one of claim 100-125, wherein the first heavy chain variable region comprises a sequence that is at least 80%, 85%, 90%, 95%, 99%, or 100% identical to SEQ ID NO: 52, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

133. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 46, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

134. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

135. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

136. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

137. The anti-PTK7/TROP2 antibody or antigen-binding fragment thereof of any one of claim 100-125, wherein the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

138. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

139. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 52, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

140. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 46, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

141. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

142. The anti-PTK7/TROP2 antibody or antigen-binding fragment thereof of any one of claim 100-125, wherein the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

143. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

144. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

145. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

146. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 52, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 74, and the second light chain variable region comprises a sequence that is at least 80%, 85%, 90%, 95%, 99%, or 100% identical to SEQ ID NO: 53.

147. The anti-PTK7/TROP2 antibody or antigen-binding fragment thereof of any one of claim 100-125, wherein the first heavy chain variable region comprises a sequence that is at least 80%, 85%, 90%, 95%, 99%, or 100% identical to SEQ ID NO: 92, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 72, and the second light chain variable region comprises a sequence that is at least 80%, 85%, 90%, 95%, 99%, or 100% identical to SEQ ID NO: 53.

148. The anti-PTK7/TROP2 antibody or antigen-binding fragment thereof of any one of claims 100-125, wherein the first heavy chain variable region comprises a sequence that is at least 80%, 85%, 90%, 95%, 99%, or 100% identical to SEQ ID NO: 92, the first light chain variable region comprises a sequence that is at least 80%, 85%, 90%, 95%, 99%, or 100% identical to SEQ ID NO: 53, the second heavy chain variable region comprises a sequence that is at least 80%, 85%, 90%, 95%, 99%, or 100% identical to SEQ ID NO: 73, and the second light chain variable region comprises a sequence that is at least 80%, 85%, 90%, 95%, 99%, or 100% identical to SEQ ID NO: 53.

149. The anti-PTK7/TROP2 antibody or antigen-binding fragment thereof of any one of claims 100-148, wherein the VH1 comprises an amino acid sequence that is at least 80%, 85%, 90%, 95%, 99%, or 100% identical to a selected VH sequence, and the VL1 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 46, and the selected VL sequence is SEQ ID NO: 53;
(2) the selected VH sequence is SEQ ID NO: 47, and the selected VL sequence is SEQ ID NO: 53;
(3) the selected VH sequence is SEQ ID NO: 48, and the selected VL sequence is SEQ ID NO: 53;
(4) the selected VH sequence is SEQ ID NO: 49, and the selected VL sequence is SEQ ID NO: 53;
(5) the selected VH sequence is SEQ ID NO: 50, and the selected VL sequence is SEQ ID NO: 53;
(6) the selected VH sequence is SEQ ID NO: 51, and the selected VL sequence is SEQ ID NO: 53;
(7) the selected VH sequence is SEQ ID NO: 52, and the selected VL sequence is SEQ ID NO: 53; and
(8) the selected VH sequence is SEQ ID NO: 92, and the selected VL sequence is SEQ ID NO: 53.

150. The anti-PTK7/TROP2 antibody or antigen-binding fragment thereof of any one of claims 100-149, wherein the VH2 comprises an amino acid sequence that is at least 80%, 85%, 90%, 95%, 99%, or 100% identical to a selected VH sequence, and the VL2 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 72, and the selected VL sequence is SEQ ID NO: 53;
(2) the selected VH sequence is SEQ ID NO: 73, and the selected VL sequence is SEQ ID NO: 53; and
(3) the selected VH sequence is SEQ ID NO: 74, and the selected VL sequence is SEQ ID NO: 53.

151. The anti-PTK7/TROP2 antibody or antigen-binding fragment thereof of any one of claims 100-150, wherein the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 46, and the selected VL sequence is SEQ ID NO: 53;
(2) the selected VH sequence is SEQ ID NO: 47, and the selected VL sequence is SEQ ID NO: 53;
(3) the selected VH sequence is SEQ ID NO: 48, and the selected VL sequence is SEQ ID NO: 53;
(4) the selected VH sequence is SEQ ID NO: 49, and the selected VL sequence is SEQ ID NO: 53;
(5) the selected VH sequence is SEQ ID NO: 50, and the selected VL sequence is SEQ ID NO: 53;
(6) the selected VH sequence is SEQ ID NO: 51, and the selected VL sequence is SEQ ID NO: 53;
(7) the selected VH sequence is SEQ ID NO: 52, and the selected VL sequence is SEQ ID NO: 53; and
(8) the selected VH sequence is SEQ ID NO: 92, and the selected VL sequence is SEQ ID NO: 53.

152. The anti-PTK7/TROP2 antibody or antigen-binding fragment thereof of any one of claims 100-151, wherein the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 72, and the selected VL sequence is SEQ ID NO: 53;
(2) the selected VH sequence is SEQ ID NO: 73, and the selected VL sequence is SEQ ID NO: 53; and
(3) the selected VH sequence is SEQ ID NO: 74, and the selected VL sequence is SEQ ID NO: 53.

153. The anti-PTK7/TROP2 antibody or antigen-binding fragment thereof of any one of claims 100-152, wherein the first antigen-binding domain specifically binds to human, monkey, or dog PTK7; and/or the second antigen-binding domain specifically binds to human, monkey, or dog TROP2.

154. The anti-PTK7/TROP2 antibody or antigen-binding fragment thereof of any one of claims 100-153, wherein the first antigen-binding domain is a human or humanized antigen-binding domain; and/or the second antigen-binding domain is a human or humanized antigen-binding domain.

155. The anti-PTK7/TROP2 antibody or antigen-binding fragment thereof of any one of claims 100-154, wherein the first antigen-binding domain is a single-chain variable fragment (scFv); and/or the second antigen-binding domain is a scFv.

156. The anti-PTK7/TROP2 antibody or antigen-binding fragment thereof of any one of claims 100-155, wherein the first light chain variable region and the second light chain variable region are identical.

157. The anti-PTK7/TROP2 antibody or antigen-binding fragment thereof of claims 100-156, wherein the antibody or antigen-binding fragment is a bispecific antibody or antigen-binding fragment thereof.

158. An antibody or antigen-binding fragment thereof that cross-competes with the antibody or antigen-binding fragment thereof of any one of claims 1-20, 52-72, and 92-157.

159. The antibody or antigen-binding fragment thereof of any one of claims 1-20, 52-72, and 92-157, wherein the antibody or antigen-binding fragment thereof comprises a fragment crystallizable region (Fc region).

160. The antibody or antigen-binding fragment thereof of claim 159, wherein the Fc region has increased complement-dependent cytotoxicity (CDC) or antibody-dependent cellular cytotoxicity (ADCC).

161. A chimeric antigen receptor (CAR) comprising the antibody or antigen-binding fragment thereof of any one of claims 1-20, 52-72, and 92-157.

162. An antibody-drug conjugate comprising the antibody or antigen-binding fragment thereof of any one of claims 1-20, 52-72, and 92-157 covalently bound to a therapeutic agent.

163. The antibody drug conjugate of claim 162, wherein the therapeutic agent is a cytotoxic or cytostatic agent.

164. The antibody drug conjugate of claim 162 or 163, wherein the therapeutic agent is MMAE or MMAF.

165. The antibody drug conjugate of claim 162 or 163, wherein the therapeutic agent is selected from

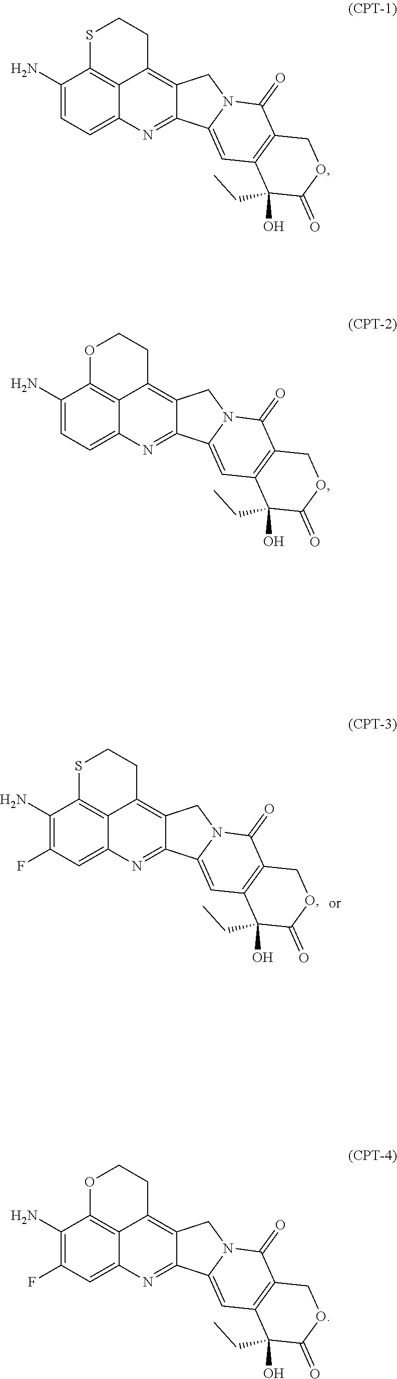 [SMILES: CC[C@@]1(O)C(=O)OCc2c1cc1n(c2=O)Cc2c-1nc1ccc(N)c3c1c2CCS3 ; CC[C@@]1(O)C(=O)OCc2c1cc1n(c2=O)Cc2c-1nc1ccc(N)c3c1c2CCO3 ; CC[C@@]1(O)C(=O)OCc2c1cc1n(c2=O)Cc2c-1nc1cc(F)c(N)c3c1c2CCS3 ; CC[C@@]1(O)C(=O)OCc2c1cc1n(c2=O)Cc2c-1nc1cc(F)c(N)c3c1c2CCO3]

166. The antibody drug conjugate of claim 162 or 165, wherein the therapeutic agent is linked to the antibody or antigen-binding fragment thereof via a linker.

167. The antibody drug conjugate of any one of claims 162, 165 and 166, wherein the linker has a structure of:

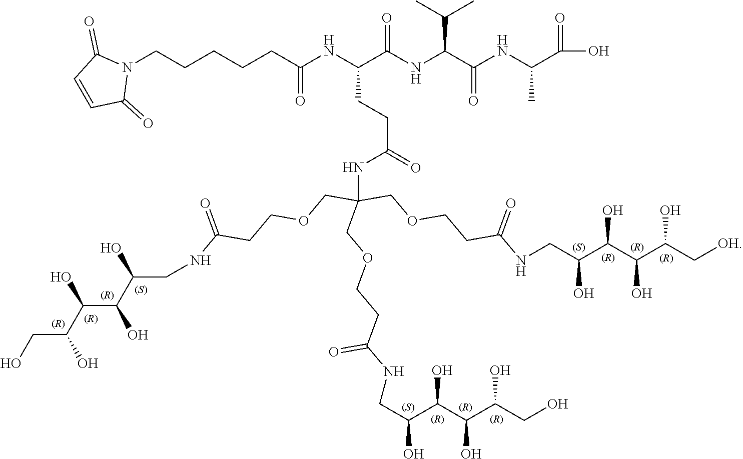

168. The antibody drug conjugate of any one of claims 162, 165-167, wherein the antibody drug conjugate has a structure of: 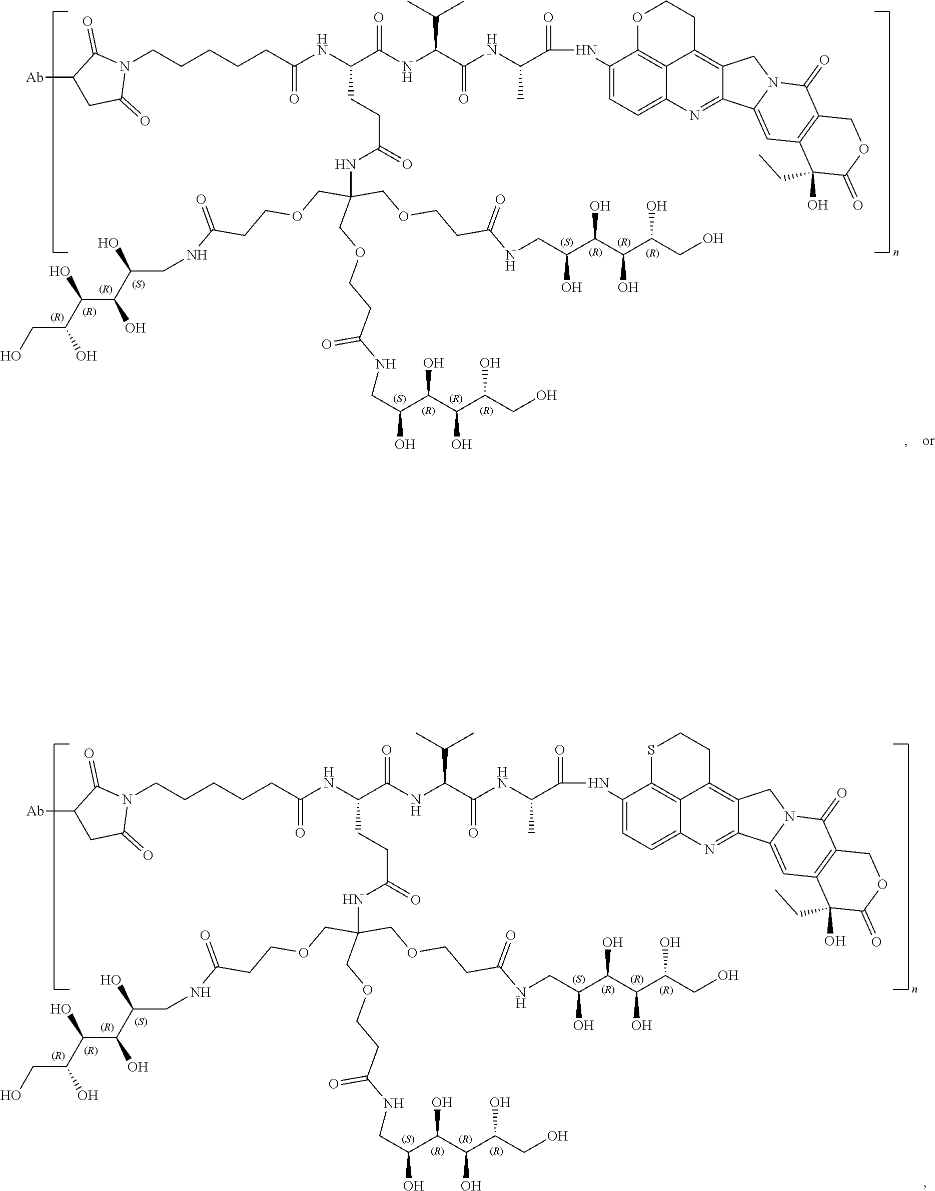
wherein n=1, 2, 3, 4, 5, 6, 7, or 8; wherein “Ab” represents the antibody or antigen-binding fragment thereof.

169. A method of treating a subject having cancer, the method comprising administering a therapeutically effective amount of a composition comprising the antibody or antigen-binding fragment thereof of any one of claims 1-20, 52-72, and 92-157, the CAR of claim 161, or the antibody-drug conjugate of any one of claims 162-168, to the subject.

170. The method of claim 169, wherein the subject has a solid tumor.

171. The method of claim 170, wherein the cancer is colon cancer, triple-negative breast cancer (TNBC), lung cancer, non-small cell lung cancer (NSCLC), pancreatic cancer, esophageal cancer, colorectal cancer, ovarian cancer (OVCA), or bladder cancer.

172. The method of any one of claims 169-171, wherein the subject is further treated with an effective amount of an anti-4-1BB antibody, an anti-OX40 antibody, an anti-PD-1 antibody, an anti-CTLA4 antibody, an anti-CD40 antibody, or an anti-PD-L1 antibody.

173. A method of decreasing the rate of tumor growth, the method comprising contacting a tumor cell with an effective amount of a composition comprising an antibody or antigen-binding fragment thereof of any one of claims 1-20, 52-72, and 92-157, the CAR of claim 161, or the antibody-drug conjugate of any one of claims 162-168.

174. A method of killing a tumor cell, the method comprising contacting a tumor cell with an effective amount of a composition comprising the antibody or antigen-binding fragment thereof of any one of claims 1-20, 52-72, and 92-157, the CAR of claim 161, or the antibody-drug conjugate of any one of claims 162-168.

175. A method of increasing immune response in a subject, the method comprising administering to the subject an effective amount of a composition comprising the antibody or antigen-binding fragment thereof of any one of claims 1-20, 52-72, and 92-157, the CAR of claim 161, or the antibody-drug conjugate of any one of claims 162-168.

176. A pharmaceutical composition comprising the antibody or antigen-binding fragment thereof of any one of claims 1-20, 52-72, and 92-157, and a pharmaceutically acceptable carrier.

177. A pharmaceutical composition comprising the antibody drug conjugate of any one of claims 162-168, and a pharmaceutically acceptable carrier.

178. The antibody-drug conjugate of any one of claims 162-168, wherein the drug-to-antibody ratio (DAR) is about 4 or 8.
